(19) United States
(12) Patent Application Publication
(10) Pub. No.: US 2024/0248028 A1
(43) Pub. Date: Jul. 25, 2024

(54) QUANTITATIVE CHIRALITY AND CONCENTRATION SENSING OF CHIRAL ANALYTES USING QUINONES, (HETERO)ARYL ISOCYANATES, AND/OR (HETERO)ARYL ISOTHIOCYANATES

(71) Applicant: GEORGETOWN UNIVERSITY, Washington, DC (US)

(72) Inventors: Christian WOLF, Arlington, VA (US); Eryn NELSON, Washington, DC (US); Jeffrey S. S. K. FORMEN, Arlington, VA (US)

(21) Appl. No.: 18/554,405

(22) PCT Filed: Feb. 9, 2022
(86) PCT No.: PCT/US2022/015775

§ 371 Date: Oct. 6, 2023

Related U.S. Application Data
(60) Provisional application No. 63/173,071, filed on Apr. 9, 2021.

(51) Int. Cl. G01N 21/33; G01N 21/19

(52) U.S. Cl. CPC G01N 21/33; G01N 21/19

(57) ABSTRACT

An analytical method is disclosed that includes the steps of: providing a sample potentially containing a chiral analyte that can exist in stereoisomeric forms; providing a probe selected from the group consisting of quinones and analogs thereof, (hetero)aryl isocyanates and analogs thereof, and (hetero)aryl isothiocyanates and analogs thereof; contacting the sample with the probe under conditions to permit covalent binding of the probe to the analyte, if present in the sample; and determining, based on any binding that occurs, the absolute configuration of the analyte in the sample and/or the concentration of the analyte in the sample and/or the enantiomeric and/or the diastereomeric composition of the analyte in the sample.

This application claims the priority benefit of U.S. Provisional Patent Application Ser. No. 63/173,071, filed Apr. 9, 2021, which is hereby incorporated by reference in its entirety.

This invention was made with government support under grant CHE-1764135 awarded by the National Science Foundation. The government has certain rights in the invention.

### FIELD OF THE DISCLOSURE

The present application relates to an analytical method for the determination of the absolute configuration and/or the concentration/yield and/or the enantiomeric/diastereomeric composition of a chiral analyte in the sample. In particular, the present method utilizes the formation of a covalent bond between an analyte in a sample and either a quinone, a (hetero)aryl isocyanate, or a (hetero)aryl isothiocyanate probe. Chiroptical and/or optical spectroscopic signal techniques are used in the analysis of the probe-analyte derivatives.

### BACKGROUND OF THE DISCLOSURE

Many important natural compounds and synthetic drugs are chiral and their enantiomers typically have individual biological functions or display different pharmacological properties. The determination of the enantiomeric composition of chiral compounds has become a fundamental task in the pharmaceutical business and also in in many other fields whenever chirality is encountered. This is particularly important in asymmetric synthesis when nonracemic products can be formed or when chiral building blocks of unknown enantiopurity need to be examined prior to use. The differentiation between enantiomers and the quantification of their ratio are, however, often very elaborate and daunting tasks.

Traditionally, enantioselective analysis is accomplished by NMR spectroscopy with the help of either a chiral solvating agent (CSA) or a chiral derivatizing agent (CDA). Alternatively, enantiomers are resolved by chromatography on a chiral stationary phase (CSP) or indirectly after derivatization with a CDA on an achiral stationary phase. For many years, chiral iso(thio)cyanates in enantiopure or enantioenriched form have been used for NMR resolution (Pirkle et al., “Dynamic NMR Studies of Diastereomeric Carbamates: Implications Toward the Determination of Relative Configuration by NMR,” 44:4891-4896 (1979); Nabeya et al., “α-Methoxy-α-(trifluoromethyl)benzyl Isocyanate. A Convenient Reagent for the Determination of the Enantiomeric Composition of Primary and Secondary Amines,” J. Org. Chem. 53:3358-3361 (1988); Sonnet et al., “Configuration Analysis of Unsaturated Hydroxy Fatty Acids,” J. Chromatogr. A 586:255-258 (1991); Kim et al., “Determination of the Optical Purity of (R)-Terbutaline by ¹H-NMR and RP-LC Using Chiral Derivatizing Agent, (S)-(−)-α-Methylbenzyl Isocyanate,” J. Pharm. Biomed. Anal., 947-956 (2001); Vodicka et al., “(S)-2-Chloro-2-fluoroethanoyl Isocyanate, a Chiral Derivative of Trichloroacetyl Isocyanate,” Chirality, 15:472-478 (2003); Vodička et al., “Synthesis of (R)- and (S)-3,3,3-Trifluoro-2-Phenylpropanoyl Isocyanates and Their Use as Reactive Analogues of Mosher's Acid,” Chirality, 17:378-387 (2005); Kaik et al., “Discrimination of Enantiomers of α-Amino Acids by Chiral Derivatizing Reagents from trans-1,2-Diaminocyclohexane,” Chirality, 20:301-306 (2008); Sabot et al., “Novel Chiral Derivatizing Isothiocyanate-Based Agent for the Enantiomeric Excess Determination of Amines,” Chem. Commun., 23:3410-3412 (2009); Wenzel and Chisholm, “Assignment of Absolute Configuration Using Chiral Reagents and NMR Spectroscopy,” Chirality, 23:190-214 (2011)) and indirect HPLC separation (Pirkle and Hoekstra, “An Example of Automated Liquid Chromatography. Synthesis of a Broad-Spectrum Resolving Agent and Resolution of 1-(1-Naphthyl)-2,2,2-Trifluoroethanol,” J. Org. Chem., 39:3904-3906 (1974); Gal, “R-α-Methylbenzyl Isothiocyanate, a New and Convenient Chiral Derivatizing Agent for the Separation of Enantiomeric Amino Compounds by High-Performance Liquid Chromatography,” J Chromatogr., 314:275-281 (1984); Dunlop and Neidle, “The Separation of D/L Amino Acid Pairs by High-Performance Liquid Chromatography after Precolumn Derivatization with Optically Active Naphthylethyl Isocyanate,” Analytical Biochemistry, 165:38-44 (1987); Martin, et al., “(−)-(S)-Flunoxaprofen and (−)-(S)-Naproxen Isocyanate: Two New Fluorescent Chiral Derivatizing Agents for an Enantiospecific Determination of Primary and Secondary Amines,” Chirality, 1:223-234 (1989); Hsu and Walters, “Chiral Separation of Ibutilide Enantiomers by Derivatization with 1-Naphthyl Isocyanate and High-Performance Liquid Chromatography on a Pirkle Column,” J. Chromatogr. A, 550:621-628 (1991); Sonnet et al., “Configuration Analysis of Unsaturated Hydroxy Fatty Acids,” J. Chromatogr., 586:255-258 (1991); Ito et al., “Resolution of the Enantiomers of Thiol Compounds by Reversed-Phase Liquid Chromatography Using Chiral Derivatization with 2,3,4,6-Tetra-O-Acetyl-β-D-Glucopyranosyl Isothiocyanate,” J. Chromatogr., 626:187-196 (1992); Bourque and Krull, “Immobilized Isocyanates for Derivatization of Amines for Chiral Recognition in Liquid Chromatography with UV Detection,” Journal of Pharmaceutical and Biomedical Analysis, 11:495-503 (1993); Lobell and Schneider, “2,3,4,6-Tetra-O-Benzoyl-β-D-Glucopyranosyl Isothiocyanate: An Efficient Reagent for the Determination of Enantiomeric Purities of Amino Acids, β-Adrenergic Blockers and Alkyloxiranes by High-Performance Liquid Chromatography Using Standard Reversed-Phase Columns,” J. Chromatogr., 633:287-294 (1993); Olsen et al., “Chiral Separations of β-Blocking Drug Substances Using Derivatization with Chiral Reagents and Normal-Phase High-Performance Liquid Chromatography,” J Chromatogr., 636:231-241 (1993); Zhou et al., “Chiral Derivatizing Reagents for Drug Enantiomers Bearing Hydroxyl Groups,” J. Chromatogr. B, 659:109-126 (1994); Kleidernigg et al., “Synthesis and Application of a New Isothiocyanate as a Chiral Derivatizing Agent for the Indirect Resolution of Chiral Amino Alcohols and Amines,” J. Chromatogr. A, 729:33-42 (1996); Peter et al., “Development of New Isothiocyanate-Based Chiral Derivatizing Agent for Amino Acids,” Chromatographia, 50:373-375 (1999); Kim et al., “Enantiomeric Purity Test of Bevantolol by Reversed-Phase High Performance Liquid Chromatography after Derivatization with 2,3,4,6-Tetra-O-Acetyl-β-D-Glucopyranosyl Isothiocyanate,” Arch. Pharm. Res., 23:568-573 (2000); Peter et al., “High-Performance Liquid Chromatographic Separation of Unusual Amino Acid Enantiomers Derivatized with (1S,2S)-1,3-Diacetoxy-1-(4-nitrophenyl)-2-propyl-Isothiocyanate,” Chromatographia Supplement, 51:148-154 (2000); Peter et al., “Liquid Chromatographic Enantioseparation of β-Blocking Agents with (1R,2R)-1,3-Diacetoxy-1-(4-nitrophenyl)-2-propyl Isothiocyanate as Chiral Derivatizing Agent,” J. Chromatogr. A, 910:247-253 (2001); Sun et al., “Chiral Derivatization Reagents for Drug Enantioseparation by High-Performance Liquid Chromatography Based upon Pre-Column Derivatization and Formation of Diastereomers: Enantioselectivity and Related Structure,” Biomed. Chromatogr. 15:116-132 (2001); Ullrich et al., “Enantioselective High-Performance Liquid Chromatography of Therapeutically Relevant Aminoalcohols as their Fluorescent 1-Naphthyl Isocyanate Derivatives,” Biomed. Chromatogr., 15:212-216 (2001); Matoga et al., “Derivatization of (±)-5-[(2-methylphenoxy)methyl]-2-amino-2-oxazoline, an Imidazoline Binding Sites Ligand, with (+)-(R)-α-Methylbenzyl Isocyanate for Drug Monitoring Purposes,” Journal of Enzyme Inhibition and Medicinal Chemistry 17:375-379 (2002); Peter and Fülöp, “Comparison of Isothiocyanate Chiral Derivatizing Reagents for High-Performance Liquid Chromatography,” Chromatographia, 56:631-636 (2002); Ko et al., “Chiral Separation of β-Blockers after Derivatization with a New Chiral Derivatization Agent, GATC,” Arch. Pharm. Res. 29:1061-1065 (2006); Ilisz and Berkecz, “Application of Chiral Derivatizing Agents in the High-Performance Liquid Chromatographic Separation of Amino Acid Enantiomers: A Review,” J. Pharmaceut. Biomed. 47:1-15 (2008); Bhushan and Batra, “High-Performance Liquid Chromatographic Enantioseparation of (RS)-Bupropion Using Isothiocyanate-Based Chiral Derivatizing Reagents,” Biomed. Chromatogr., 27:956-959 (2013); Escrig-Doménech et al., “Derivatization of Hydroxy Functional Groups for Liquid Chromatography and Capillary Electroseparation,” J. Chromatogr. A, 1296:140-156 (2013)) of the enantiomers of amines, alcohols, amino alcohols, hydroxy esters and amino acids that were converted to distinguishable diastereomeric (thio)urea or (thio)carbamate mixtures to achieve spectroscopic or chromatographic resolution. While these methods are well-established and often provide sufficient enantiomeric resolution, they do not satisfy the increasing demand for parallel data acquisition and high-throughput screening capacities. The time usually required for chromatographic enantio separations is not acceptable when large numbers of samples need to be examined. Both NMR and chromatography are inherently serial techniques, which means only one sample can be analyzed at a time. The necessary use of an enantiopure chiral reagent, additive or CSP can increase costs and preparation time. Furthermore, false results are obtained with CDAs that are not perfectly enantiomerically pure (Wolf, “Dynamic Stereochemistry of Chiral Compounds—Principles and Applications,” RSC, Cambridge, UK, 136-179 (2008), which is hereby incorporated by reference in its entirety).

Quinones are ubiquitous small molecules in nature and have found widespread use in numerous applications that are testimony to the unique physiological, medicinal, photophysical and chemical properties of this fascinating pool of compounds. They play essential roles in important biological processes including aerobic respiration (Anand et al., “Adaptive Evolution Reveals a Tradeoff Between Growth Rate and Oxidative Stress During Naphthoquinone-Based Aerobic Respiration,” Proc. Natl. Acad Sci. USA 116:25287-25292 (2019)), photosynthesis (Rabenstein et al., “Electron Transfer Between the Quinones in the Photosynthetic Reaction Center and Its Coupling to Conformational Changes,” Biochemistry 39(34):10487-10496 (2000)), cellular signaling (Ji et al., “Molecular Mechanism of Quinone Signaling Mediated Through S-quinonization of a YodB Family Repressor QsrR,” Proc. Natl. Acad Sci. USA, 110:5010-5015 (2013)), and metabolic transformations (Malpica et al., “Identification of a Quinone-sensitive Redox Switch in the ArcB Sensor Kinase,” Proc. Natl. Acad Sci. USA 101(36):13318-13323 (2004)), and embody a frequently encountered structural unit in Vitamin K (Vos et al., “Vitamin K2 is a Mitochondrial Electron Carrier that Rescues Pink1 Deficiency,” Science 336 (6086):1306-1310 (2012)), Coenzyme Q₁₀ (Ernster and Dallner, “Biochemical, Physiological and Medical Aspects of Ubiquinone Function,” Biochim Biophys Acta, 1271(1):195-204 (1995)), Menadione (Buc Calderon et al., “Taper, H. S. Potential Therapeutic Application of the Association of Vitamins C and K3 in Cancer Treatment,” Curr. Med Chem. 9:2271-2285 (2002)), Streptonigrin (Rao et al., “The Structure of Streptonigrin,” J Am. Chem. Soc. 85:2532-2533 (1963)), Prekinamycin (Gould et al., “Identification of Prekinamycin in Extracts of Streptomyces murayamaensis,”. Org. Chem. 61:5720-5721 (1996)) and other biologically active compounds (Asche, “Antitumour Quinones,” Mini. Rev. Med. Chem. 5:449-467 (2005)). Quinones also serve as versatile chemical building blocks (Hosamani et al., “Catalytic Asymmetric Reactions and Synthesis of Quinones,” Org. Biomol. Chem., 14:6913-6931 (2016)), dyes (Dias et al., “Quinone-based Fluorophores for Imaging Biological Processes,” Chem. Soc. Rev. 47:12-27 (2018)), redox catalysts (Wendlandt and Stahl, “Quinone-Catalyzed Selective Oxidation of Organic Molecules,” Angew. Chem., Int. Ed., 54:14638-14658 (2015)), and are used in energy harvesting (Cheng et al., Angew. Chem., Int. Ed., 51:9896-9899 (2012)), electrochemical water-splitting (Rausch et al., J. Am. Chem. Soc., 135:13656-13659 (2013)), and metal-free energy storage (Huskinson et al., Nature, 505:195 (2014)) technologies. To date, they have not but used for high-throughput enantiomeric resolution.

In recent years, optical methods such as circular dichroism (CD) spectroscopy have become a popular, cost-effective alternative for enantiomeric ratio (er) determination. The simplicity of CD analysis combined with the possibility of fast and parallel data collection at minimal solvent usage can reduce the workload and increase sample throughput. Several examples demonstrating the efficacy of chiroptical er determination with a wide variety of substrates have been reported (Nieto et al., “High-Throughput Screening of Identity, Enantiomeric Excess, and Concentration Using MLCT Transitions in CD Spectroscopy,” J. Am. Chem. Soc., 130:9232-9233 (2008); Nieto et al., “Rapid Enantiomeric Excess and Concentration Determination Using Simple Racemic Metal Complexes,” Org. Lett., 10:5167-5170 (2008); Ghosn and Wolf, “Chiral Amplification with a Stereodynamic Triaryl Probe: Assignment of the Absolute Configuration and Enantiomeric Excess of Amino Alcohols,” J. Am. Chem. Soc., 131:16360-16361 (2009); Nieto et al., “A Facile Circular Dichroism Protocol for Rapid Determination of Enantiomeric Excess and Concentration of Chiral Primary Amines,” Chem. Eur. J., 16:227-232 (2010); Joyce et al., “A Simple Method for the Determination of Enantiomeric Excess and Identity of Chiral Carboxylic Acids,” J. Am. Chem. Soc., 133:13746-13752 (2011); Leung and Anslyn, “Rapid Determination of Enantiomeric Excess of α-Chiral Cyclohexanones Using Circular Dichroism Spectroscopy,” Org. Lett., 13:2298-2301 (2011); Dragna et al., “In Situ Assembly of Octahedral Fe(II) Complexes for the Enantiomeric Excess Determination of Chiral Amines Using Circular Dichroism Spectroscopy,” J. Am. Chem. Soc., 134:4398-4407 (2012); Leung et al., “Rapid Determination of Enantiomeric Excess: A Focus on Optical Approaches,” Chem. Soc. Rev., 41:448-479 (2012); You et al., “An Exciton-Coupled Circular Dichroism Protocol for the Determination of Identity, Chirality, and Enantiomeric Excess of Chiral Secondary Alcohols,” J. Am. Chem. Soc., 134:7117-7125 (2012); Bentley et al., “Chirality Sensing of Amines, Diamines, Amino Acids, Amino Alcohols, and α-Hydroxy Acids with a Single Probe,” J. Am. Chem. Soc., 135:18052-18055 (2013); Bentley and Wolf, “Comprehensive Chirality Sensing: Development of Stereodynamic Probes with a Dual (Chir)Optical Response,” J. Org. Chem., 79:6517-6531 (2014); Jo et al., “Rapid Optical Methods for Enantiomeric Excess Analysis: From Enantioselective Indicator Displacement Assays to Exciton-Coupled Circular Dichroism,” Acc. Chem. Res., 47:2212-2221 (2014); Metola et al., “Well Plate Circular Dichroism Reader for the Rapid Determination of Enantiomeric Excess,” Chem. Sci., 5:4278-4282 (2014); Herrera et al., “Optical Analysis of Reaction Yield and Enantiomeric Excess: A New Paradigm Ready for Prime Time,” J. Am. Chem. Soc., 140:10385-10401 (2018); Thanzeel et al., “Quantitative Chiroptical Sensing of Free Amino Acids, Biothiols, Amines, and Amino Alcohols with an Aryl Fluoride Probe,” J. Am. Chem. Soc., 141:16382-16387 (2019)). Because CD spectroscopy is inherently primed to distinguish between enantiomers, it eliminates the need for CSAs and CDAs, and it allows combined determination of the enantiomeric composition and total concentration of a chiral sample if used in conjunction with UV spectroscopy, a task that is easily accomplished with modern spectrophotometers.

However, what is lacking in the art is a suitable set of probe molecules—and sensing assay using those probe molecules, that are compatible with CD spectroscopy, capable of smoothly reacting with a wide variety of analytes including amines, amino alcohols, amino acids, diols and the notoriously challenging class of alcohols, and affording reliable results for quantitative er and concentration analysis.

The present invention is directed to overcoming these and other deficiencies in the art.

### SUMMARY

A first aspect of the disclosure relates to an analytical method that includes the steps of: providing a sample potentially containing a chiral analyte that can exist in stereoisomeric forms; providing a probe selected from the group consisting of quinones and analogs thereof, (hetero)aryl isocyanates and analogs thereof, and (hetero)aryl isothiocyanates and analogs thereof, contacting the sample with the probe under conditions to permit covalent binding of the probe to the analyte, if present in the sample; and determining, based on any binding that occurs, the absolute configuration of the analyte in the sample and/or the concentration of the analyte in the sample and/or the enantiomeric and/or the diastereomeric composition of the analyte in the sample.

According to one embodiment, the quinone probe (and analogs thereof) has the structure according to Formula I:

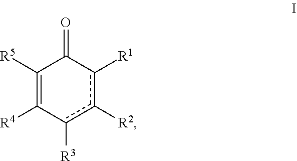

wherein:
R¹ and R³ are independently selected from the group consisting of ═O, X, halogen, —CN, —NO₂, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
R², R⁴, and R⁵ are independently selected from the group consisting of X, halogen, —CN, —C₁-C₆ alkyl, —C₁-C₆ alkoxy, —NO₂, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and
X is a leaving group selected from halogen, —CN, —OR_{b}, —OC(O)R_{b}, —OS(O)₂R_{b}, —S(O)₂—O—R_{b}, —N₂⁺, —N⁺(R_{b})₃, —S⁺(R_{b})₂, and —P⁺(R_{b})₃; wherein each R_{b} is independently selected from the group consisting of -alkyl, —O-alkyl, —N-alkyl, -alkenyl, -alkynyl, -perfluoroalkyl, -perfluoroalkenyl, -perfluoroalkynyl, -aryl, -perfluoroaryl, —O-aryl, —N-aryl, —O-perfluoroaryl, —N-perfluoroaryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
wherein, optionally, R¹ and R², R² and R³, R³ and R⁴, and/or R⁴ and R⁵ are alternatively taken together with the carbon atoms to which they are attached to form a monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{c}, —O—C(O)R_{C}, —NHC(O)R_{c}, —NR_{c}C(O)R_{c}, —NO₂, —CN, -halogen, and —SO₂R_{c}, wherein each R_{c} is independently Ar, alkyl, or CH₂Ar and Ar is an aryl or heteroaryl;
with the proviso that one of R¹ or R³ is ═O, and at least one of R¹, R², R³, R⁴, or R⁵ is X.

According to another embodiment, the (hetero)aryl isocyanates and (hetero)aryl isothiocyanates (and analogs thereof) have the structure according to Formula II, IIa, or IIb.

The (hetero)aryl isocyanate or (hetero)aryl isothiocyanate (or an analog thereof) of Formula II has the structure:

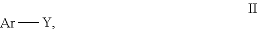

wherein:
Y is —NCO or —NCS; and
Ar is a substituted or unsubstituted aromatic or heteroaromatic chromophore.

The (hetero)aryl isocyanate or (hetero)aryl isothiocyanate (or an analog thereof) of Formula IIa has the structure:

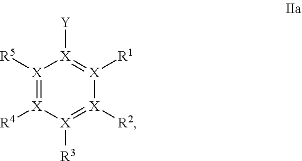

wherein:
Y is —NCO or —NCS;
each X is independently C or N; and
R¹, R², R³, R⁴, and R⁵ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ;
wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and
wherein, optionally, R¹ and R², R² and R³, R³ and R⁴, and/or R⁴ and R⁵ are alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

The (hetero)aryl isocyanate or (hetero)aryl isothiocyanate (or an analog thereof) of Formula IIb has the structure:

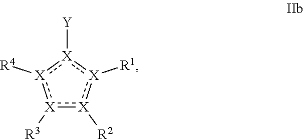

wherein:
Y is —NCO or —NCS;
each X is independently C, N, O or S; and
R¹, R², R³, and R⁴ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ;
wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
wherein no more than one of R¹, R², R³, or R⁴, is Y; and
wherein, optionally, R¹ and R², R² and R³, and/or R³ and R⁴ is alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

Disclosed herein and demonstrated in the accompanying Examples are inexpensive, commercially available achiral quinone and aryliso(thio)cyanate sensors that smoothly react with amino and alcohol groups under mild conditions toward products exhibiting characteristic UV and CD signals above 300 nm which were used for quantitative er and concentration analysis, as illustrated in FIG. 1. Quinone sensors yield di-substituted products displaying two antiparallel push-pull conjugates, and the aryliso(thio)cyanate sensors form (thio)urea or (thio)carbamate products. The optical sensing assay was easily performed and broadly applicable to amines, amino alcohols, amino acids, diols and the notoriously challenging class of alcohols, which was demonstrated with a large number of examples. Cumbersome work-up procedures and the risk of producing false results due to enantiomeric impurities or kinetic resolution effects when CDAs need to be used are avoided. At the same time, the in situ generation of distinct UV and CD signals at high wavelengths overcomes issues with insufficient resolution of overlapping signals often encountered with chiral NMR solvating agents that rely on weak binding forces rather than covalent bond formation. In contrast to inherently serial, traditional NMR and chromatographic methods that may consume large amounts of solvents, this optical sensing assay was operationally simple, cost-effective and amenable to high-throughput experimentation where many samples can be screened in parallel. This has far-reaching implications and potential to streamline chiral compound development projects in numerous laboratories.

### BRIEF DESCRIPTION OF THE DRAWINGS

FIG. 1 is a schematic depiction for the use of an arylisocyanate probe for quantitative concentration and er determination of chiral compounds.

FIGS. 2A-2B shows structures of arylisocyanate and arylisothiocyanate sensors 1-8 (FIG. 2A) and comparison of the chiroptical responses of 1, 5, and 6 to the enantiomers of 1-phenylethylamine (FIG. 2B). The CD spectrum obtained with free (R)-9 under the same conditions but in the absence of a sensor is shown in orange. Conditions: 1-Phenylethylamine (82.5 mM) and an iso(thio)cyanate sensor (99.0 mM) were mixed in 1.0 mL of CHCl₃ and the reaction was stirred overnight followed by dilution with acetonitrile to 0.37-0.50 mM prior to CD analysis.

FIGS. 3A-3B show the structures of chiral amines and amino acids tested (FIG. 3A), and selected CD spectra observed upon binding of 11, 17, 37 and 43, respectively, to 1 (FIG. 3B). The CD analyses of the reactions with the amines were conducted at 0.58-0.65 mM in acetonitrile and for the amino acid derived ureas at 0.41 mM in aqueous acetonitrile.

FIGS. 4A-4F show catalyst screening to achieve quantitative alcohol conversion with the isocyanate probe 1 at room temperature. FIGS. 4A-4D: (R)-Phenylethanol (81.9 mM), sensor 1 (98.2 mM) and catalyst (0.2 equivalents, 16.4 mM) were mixed in 1.0 mL of CDCl₃ and the reaction was stirred overnight. FIG. 4E is a reference reaction without catalyst. FIG. 4F is a NMR spectrum of 1-phenylethanol. Peaks marked with • on the X-axis in FIGS. 4A-4F correspond to hydrogens marked with • in the scheme above. •(1): peak around 4.9; •(2): peak around 1.4; •(3): peak around 5.9; •(4): peak around 1.7.

FIGS. 5A-B are the X-ray structures of (S)-1-(2-nitrophenyl)-3-(1-phenylethyl)urea (5A) and (R)-1-(naphthalen-2-yl)ethyl (2-nitrophenyl)carbamate (5B).

FIGS. 6A-6D shows the amino alcohol and alcohol structures (FIG. 6A), selected chiroptical sensing results (FIG. 6B), linear CD response of 1 at 350 nm to the % ee of alcohol 31 (FIG. 6C), and the UV response of 1 at 350 nm to the concentration of alcohol 31 (FIG. 6D). FIG. 6A depicts the structures of alcohols and amino alcohols tested. FIG. 6B shows selected CD spectra observed upon binding of 21, 24, 27 and 36, respectively, to 1. Two equivalents of 1 were used for the amino alcohol sensing. FIG. 6C shows the linear chiroptical response of sensor 1 to nonracemic samples of 1-(2-naphthyl)ethanol. FIG. 6D shows the UV response to alcohol 31. The CD analyses of the reactions with the amino alcohols were conducted at 0.17-0.28 mM and for the alcohol derived carbamates at 0.13-0.82 mM in acetonitrile.

FIGS. 7A-7B shows the correlation of the UV (FIG. 7A) and CD sensor responses (FIG. 7B) to the concentration and enantiomeric composition of 1-phenylethylamine. The UV and CD measurements were performed after dilution with acetonitrile.

FIG. 8 shows the reaction for chiroptical sensing of phenylethylamine with sensors 1-8.

FIG. 9 shows individual CD concentrations were as follows: 1 (0.50 mM), 2 (0.12 mM), 3 (0.74 mM), 4 (0.37 mM), 5 (0.37 mM), 6 (0.50 mM), 7 (0.10 mM), 8 (1.24 mM). Concentrations were adjusted to avoid HT >600. 1-Phenylethylamine (82.5 mM) and an iso(thio)cyanate sensors 1-8 (99.0 mM) were mixed in 1.0 mL of CHCl₃ and the reaction was stirred overnight followed by CD analysis in 2.0 mL of ACN (0.10-1.24 mM). The urea derived from sensor 1 yielded the strongest red-shifted CD signal. Thus, further reaction optimization was carried out with sensor 1.

FIG. 10 shows NMR analysis of the reaction between 1 and (R)-phenylethanol. (R)-Phenylethanol (81.9 mM) and sensor 1 (98.2 mM) were mixed in 1.0 mL of CDCl₃ with 0.2 equivalents of DMAP (16.4 mM) and the reaction was monitored by ¹H NMR. The reaction was complete within 1.5 hours.

FIG. 11. NMR analysis of the reaction between 1 and (S)-phenylethylamine. (S)-Phenylethylamine (82.5 mM) and sensor 1 (99.0 mM) were mixed in 1.0 mL of CDCl₃ and the reaction was monitored by ¹H NMR. The reaction was complete within 15 minutes.

FIG. 12 is CD spectra of the solvent screening reactions run in CHCl₃. (S)-Phenylethylamine (82.5 mM) and sensor 1 (99.0 mM) were mixed for 15 minutes in 1.0 mL of CHCl₃ then diluted to a final volume of 2.0 mL with ACN, CHCl₃, EtOH, or hexanes (0.41 mM) for CD analysis. The reaction run in CHCl₃ followed by dilution with ACN yielded the strongest CD signal. All subsequent reactions were thus run in CHCl₃ and diluted with ACN for CD analysis.

FIG. 13 is solvent screening reactions run in ACN. (S)-Phenylethylamine (82.5 mM) and sensor 1 (99.0 mM) were mixed for 15 minutes in 1.0 mL of ACN, then diluted to a final volume of 2.0 mL with ACN, CHCl₃, EtOH, or hexanes (0.41 mM) for CD analysis. The reaction run in CHCl₃ followed by dilution with ACN yielded the strongest CD signal. All subsequent reactions were thus run in CHCl₃ and diluted with ACN for CD analysis.

FIG. 14 shows the amine substrate scope. A solution of chiral amines 9-17 (45-85 mM) and sensor 1 (1.2 equivalents) in 1.0 mL of chloroform was stirred for 15 minutes. For chiral diamine 18, 2.4 equivalents of the sensor were used. CD analysis was performed after dilution with ACN (20.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation.

FIG. 15 is a CD spectra obtained by applying sensor 1 to (R)-9 (blue) and (S)-9 (red). CD measurements were taken at 0.83 mM in acetonitrile.

FIG. 16 is a CD spectra obtained by applying sensor 1 to (R)-10 (blue) and (S)-10 (red)CD measurements were taken at 0.74 mM in acetonitrile.

FIG. 17 is a CD spectra obtained by applying sensor 1 to (R)-11 (blue) and (S)-11 (red). CD measurements were taken at 0.58 mM in acetonitrile.

FIG. 18 is a CD spectra obtained by applying sensor 1 to (R)-12 (blue) and (S)-12 (red). CD measurements were taken at 0.74 mM in acetonitrile.

FIG. 19 is a CD spectra obtained by applying sensor 1 to (R)-13 (blue) and (S)-13 (red). CD measurements were taken at 0.58 mM in acetonitrile.

FIG. 20 is a CD spectra obtained by applying sensor 1 to (R)-14 (blue) and (S)-14 (red). CD measurements were taken at 0.47 mM in acetonitrile.

FIG. 21 is a CD spectra obtained by applying sensor 1 to (R)-15 (blue) and (S)-15 (red). CD measurements were taken at 0.68 mM in acetonitrile.

FIG. 22 is a CD spectra obtained by applying sensor 1 to (R)-16 (blue) and (S)-16 (red). CD measurements were taken at 0.79 mM in acetonitrile.

FIG. 23 is a CD spectra obtained by applying sensor 1 to (R)-17 (blue) and (S)-17 (red). CD measurements were taken at 0.65 mM in acetonitrile.

FIG. 24 is a CD spectra obtained by applying sensor 1 to (R,R)-18 (blue) and (S,S)-18 (red). CD measurements were taken at 0.88 mM in acetonitrile.

FIG. 25 depicts the amino alcohol substrate scope. In a glovebox, a solution of chiral amino alcohols 19-26 (27-100 mM), sensor 1 (2.4 equivalents), and DMAP (0.2 equivalents) in 2.0 mL of chloroform was stirred overnight. CD analysis was performed after dilution with ACN to the final concentration indicated under each Figure (15.0-40.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation.

FIG. 26 is a CD spectra obtained by applying sensor 1 to (R)-19 (blue) and (S)-19 (red). CD measurements were taken at 0.36 mM in acetonitrile.

FIG. 27 is a CD spectra obtained by applying sensor 1 to (1R,2S)-20 (blue) and (1S,2R)-20 (red). CD measurements were taken at 0.36 mM in acetonitrile.

FIG. 28 is a CD spectra obtained by applying sensor 1 to (1R,2S)-21 (blue) and (1S,2R)-21 (red). CD measurements were taken at 0.34 mM in acetonitrile.

FIG. 29 is a CD spectra obtained by applying sensor 1 to (R)-22 (blue) and (S)-22 (red). CD measurements were taken at 0.49 mM in acetonitrile.

FIG. 30 is a CD spectra obtained by applying sensor 1 to (R)-23 (blue) and (S)-23 (red). CD measurements were taken at 0.39 mM in acetonitrile.

FIG. 31 is a CD spectra obtained by applying sensor 1 to (R)-24 (blue) and (S)-24 (red). CD measurements were taken at 0.39 mM in acetonitrile.

FIG. 32 is a CD spectra obtained by applying sensor 1 to (R)-25 (blue) and (S)-25 (red). CD measurements were taken at 0.37 mM in acetonitrile.

FIG. 33 is a CD spectra obtained by applying sensor 1 to (1R,2R)-26 (blue) and (1S,2S)-26 (red). CD measurements were taken at 0.45 mM in acetonitrile.

FIG. 34 is the alcohol substrate scope. In a glovebox, a solution of chiral alcohols 27-36 (70-130 mM), sensor 1, and DMAP (0.2 equivalents) in 1.0 mL of chloroform was stirred overnight. For chiral alcohols 27-33, 1.2 equivalents of the sensor were used; for diols 34-36, 2.4 equivalents of the sensor were used. CD analysis was performed after dilution with ACN to the final concentration indicated under each Figure (5.0-20.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation.

FIG. 35 is a CD spectra obtained by applying sensor 1 to (R)-27 (blue) and (S)-27 (red). CD measurements were taken at 0.50 mM in acetonitrile.

FIG. 36 is a CD spectra obtained by applying sensor 1 to (R)-28 (blue) and (S)-28 (red). CD measurements were taken at 0.50 mM in acetonitrile.

FIG. 37 is a CD spectra obtained by applying sensor 1 to (R)-29 (blue) and (S)-29 (red). CD measurements were taken at 0.50 mM in acetonitrile.

FIG. 38 is a CD spectra obtained by applying sensor 1 to (R)-30 (blue) and (S)-30 (red). CD measurements were taken at 0.50 mM in acetonitrile.

FIG. 39 is a CD spectra obtained by applying sensor 1 to (R)-31 (blue) and (S)-31 (red). CD measurements were taken at 0.75 mM in acetonitrile.

FIG. 40 is a CD spectra obtained by applying sensor 1 to (1R,2S)-32 (blue) and (1S,2R)-32 (red). CD measurements were taken at 0.60 mM in acetonitrile.

FIG. 41 is a CD spectra obtained by applying sensor 1 to (R)-33 (blue) and (S)-33 (red). CD measurements were taken at 0.71 mM in acetonitrile.

FIG. 42 is a CD spectra obtained by applying sensor 1 to (1R,2R)-34 (blue) and (1S,2S)-34 (red). CD measurements were taken at 0.27 mM in acetonitrile.

FIG. 43 is a CD spectra obtained by applying sensor 1 to (2R,4R)-35 (blue) and (2S,4S)-35 (red). CD measurements were taken at 0.34 mM in acetonitrile.

FIG. 44 is a CD spectra obtained by applying sensor 1 to (2R,5R)-36 (blue) and (2S,5S)-36 (red). CD measurements were taken at 0.27 mM in acetonitrile.

FIG. 45 is the amino acid substrate scope. A solution of amino acids 37-43 (9.0 mM), sensor 1 (10.8 or 21.6 mM), and Na₂CO₃ (18.0 mM) in 1.0 mL of a 4:1 ACN:water mixture was stirred overnight. For amino acids 37 and 40-43, 1.2 equivalents of the sensor (10.8 mM) were used. For amino acids 38 and 39, 2.4 equivalents of the sensor (21.6 mM) were used. For tyrosine (38), DMAP (0.2 equivalents) was used as a catalyst. CD analysis was performed after dilution with ACN to the final concentration indicated under each Figure (50.0-150.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation.

FIG. 46 is a CD spectra obtained by applying sensor 1 to (R)-37 (blue) and (S)-37 (red). CD measurements were taken at 0.41 mM in acetonitrile.

FIG. 47 is a CD spectra obtained by applying sensor 1 to (R)-38 (blue) and (S)-38 (red). CD measurements were taken at 0.23 mM in acetonitrile.

FIG. 48 is a CD spectra obtained by applying sensor 1 to (R)-39 (blue) and (S)-39 (red). CD measurements were taken at 0.41 mM in acetonitrile.

FIG. 49 is a CD spectra obtained by applying sensor 1 to (R)-40 (blue) and (S)-40 (red). CD measurements were taken at 0.41 mM in acetonitrile.

FIG. 50 is a CD spectra obtained by applying sensor 1 to (R)-41 (blue) and (S)-41 (red). CD measurements were taken at 0.41 mM in acetonitrile.

FIG. 51 is a CD spectra obtained by applying sensor 1 to (R)-42 (blue) and (S)-42 (red). CD measurements were taken at 0.68 mM in acetonitrile.

FIG. 52 is a CD spectra obtained by applying sensor 1 to (R)-43 (blue) and (S)-43 (red). CD measurements were taken at 0.41 mM in acetonitrile.

FIGS. 53A-C shows sensor and test substrate structures (FIG. 53A), sensing concept and reaction analysis (FIG. 53B), and chiroptical response (FIG. 53C). FIG. 53A shows structures of the quinone sensors 101-104 and (S)-phenylethylamine, 105. Lines around 350 nm correspond, from top to bottom, to: 105, 104+105, 103+105, 102+105, and 101+105. FIG. 53B shows sensing strategy and reaction analysis including crystal structures of (S,S)-2,5-difluoro-3,6-bis((1-phenylethyl)amino)-1,4-benzoquinone and (S,S)-2-chloro-5-cyano-3,6-bis((1-phenylethyl)amino)-1,4-benzoquinone. FIG. 53C shows circular dichroism spectra of the substitution products obtained from 101-104 and 105 at 0.13 mM in acetonitrile. The lack of a CD signal of free (S)-105 under the same conditions is shown for comparison.

FIGS. 54A-B shows the structures of chiral amines tested (only one enantiomer is shown, FIG. 54A) and selected CD spectra obtained by sensing of 107, 109, 113 and 115, respectively, with 101 in THF (FIG. 54B). The CD analyses of the reactions with these amines were conducted at 0.10-0.15 mM in THF.

FIGS. 55A-B shows structures of chiral amino alcohols and amino acids tested (only one enantiomer is shown, FIG. 55A) and selected CD spectra obtained by sensing with 101 in THF or aqueous borate pH 8.5 buffer (FIG. 55B). The CD analyses of the reactions with the amino alcohols 119 and 127 were conducted at 0.10-0.15 mM in THF. The amino acid sensing was performed at 0.07 mM in ACN:aqueous pH 8.5 borate buffer 4:1 (v/v).

FIGS. 56A-B shows the correlation of the UV and CD sensor responses of 101 (FIG. 56A) to the concentration and enantiomeric composition of 1-phenylethylamine (FIG. 56B). The UV and CD measurements were performed after dilution with THE as described above.

FIGS. 57A-C shows chiroptical redox switching using NaBH₄ and air as external stimuli. FIG. 57A shows reversible redox chemistry. FIG. 57B shows CD cycles. FIG. 57C shows UV cycles.

FIG. 58 shows chiroptical sensing of (S)-phenylethylamine with quinones 101-104. (S)-1-Phenylethylamine (3.33 mM), Et₃N (3.33 mM) and a quinone sensor 101-104 (1.67 mM) were mixed in 1.5 mL of ACN and the reaction was stirred overnight followed by CD analysis in 2.0 mL of ACN (0.13 mM).

FIG. 59 shows chiroptical sensing of (S)-phenylethylamine with quinones 101-104. Sensor 101 yielded the strongest CD signal. Thus, further optimization was carried out with sensor 101. CD measurements were taken at 0.13 mM.

FIG. 60 shows the NMR analysis of the reaction between 101 and (S)-phenylethylamine. A solution of racemic phenylethylamine (33.3 mM), Et₃N (33.3 mM) and sensor 101 (16.7 mM) in 1.5 mL of CD₃CN was stirred for 5 minutes and analyzed by ¹H NMR spectroscopy. The reaction produces approximately a 1:2:1 ratio of the (R,R)-, (R,S)- and the (S,S)-2,5-difluoro-3,6-bis((1-phenylethyl)amino)-1,4-benzoquinone.

FIG. 61 shows the NMR analysis of the reaction between 101 and racemic phenylethylamine.

FIG. 62 shows CD spectra of the reactions run in CHCl₃, followed by dilution in respective solvent. CD measurements were taken at 0.15 mM. (S)-1-Phenylethylamine (33.3 mM), Et₃N (33.3 mM) and sensor 1 (16.7 mM) were mixed for 5 minutes in either 1.5 mL of THF, 1.5 mL of ACN or 1.5 mL of CHCl₃, then diluted to a final volume of 2.0 mL with ACN, CHCl₃, THF or EtOH to obtain a final concentration of 0.15 mM for CD analysis. Reactions run in ACN or THE gave stronger CD signals than reactions run in CHCl₃. Differences in CD signal were negligible between dilution solvents. Strong CD spectra were obtained when THE was used as the reaction and the dilution solvent. All subsequent reactions were thus conducted using THF as solvent unless noted otherwise.

FIG. 63 shows CD spectra of the reactions run in ACN, followed by dilution in respective solvent. CD measurements were taken at 0.15 mM.

FIG. 64 shows CD spectra of the reactions run in THF, followed by dilution in respective solvent. CD measurements were taken at 0.15 mM.

FIG. 65 shows the amine substrate scope. A solution of chiral amine 105-118 (3.33 mM), Et₃N (3.33 mM) and sensor 101 (1.67 mM) in 1.5 mL of tetrahydrofuran was stirred for 5 minutes. CD analysis was performed after dilution with THE to the final concentration indicated under each figure (125-175 μL of the reaction mixture were diluted to 2.0 mL THF).

FIG. 66 shows the CD spectra obtained by applying sensor 101 to (R)-105 (blue) and (S)-105 (red). CD measurements were taken at 0.15 mM in tetrahydrofuran.

FIG. 67 shows the CD spectra obtained by applying sensor 101 to (R)-106 (blue) and (S)-106 (red). CD measurements were taken at 0.10 mM in tetrahydrofuran.

FIG. 68 shows the CD spectra obtained by applying sensor 101 to (R)-107 (blue) and (S)-107 (red). CD measurements were taken at 0.10 mM in tetrahydrofuran.

FIG. 69 shows the CD spectra obtained by applying sensor 101 to (R)-108 (blue) and (S)-108 (red). CD measurements were taken at 0.10 mM in tetrahydrofuran.

FIG. 70 shows the CD spectra obtained by applying sensor 101 to (R)-109 (blue) and (S)-109 (red). CD measurements were taken at 0.10 mM in tetrahydrofuran.

FIG. 71 shows the CD spectra obtained by applying sensor 101 to (R)-110 (blue) and (S)-110 (red). CD measurements were taken at 0.10 mM in tetrahydrofuran.

FIG. 72 shows the CD spectra obtained by applying sensor 101 to (R)-111 (blue) and (S)-111 (red). CD measurements were taken at 0.10 mM in tetrahydrofuran.

FIG. 73 shows the CD spectra obtained by applying sensor 101 to (R)-112 (blue) and (S)-112 (red). CD measurements were taken at 0.10 mM in tetrahydrofuran.

FIG. 74 shows the CD spectra obtained by applying sensor 101 to (R)-113 (blue) and (S)-113 (red). CD measurements were taken at 0.10 mM in tetrahydrofuran.

FIG. 75 shows the CD spectra obtained by applying sensor 101 to (R,R)-114 (blue) and (S,S)-114 (red). CD measurements were taken at 0.10 mM in tetrahydrofuran.

FIG. 76 shows the CD spectra obtained by applying sensor 101 to (R)-115 (blue) and (S)-115 (red). CD measurements were taken at 0.17 mM in tetrahydrofuran.

FIG. 77 shows the CD spectra obtained by applying sensor 101 to (R)-116 (blue) and (S)-116 (red). CD measurements were taken at 0.15 mM in tetrahydrofuran.

FIG. 78 shows the CD spectra obtained by applying sensor 101 to (R)-117 (blue) and (S)-117 (red). CD measurements were taken at 0.10 mM in tetrahydrofuran.

FIG. 79 shows the CD spectra obtained by applying sensor 101 to (R)-118 (blue) and (S)-118 (red). CD measurements were taken at 0.10 mM in tetrahydrofuran.

FIG. 80 shows the amino alcohol substrate scope. A solution of a chiral amino alcohol 119-127 (3.33 mM), Et₃N (3.33 mM) and sensor 101 (1.67 mM) in 1.5 mL of tetrahydrofuran was stirred for 5 minutes. CD analysis was performed after dilution with THE to the final concentration indicated under each Figure (125-200 μL of the reaction mixture were added to 2.0 mL THF).

FIG. 81 shows the CD spectra obtained by applying sensor 101 to (R)-119 (blue) and (S)-119 (red). CD measurements were taken at 0.15 mM in tetrahydrofuran.

FIG. 82 shows the CD spectra obtained by applying sensor 101 to (R)-120 (blue) and (S)-120 (red). measurements were taken at 0.13 mM in tetrahydrofuran.

FIG. 83 shows the CD spectra obtained by applying sensor 101 to (R,S)-121 (blue) and (S,R)-121 (red). CD measurements were taken at 0.15 mM in tetrahydrofuran.

FIG. 84 shows the CD spectra obtained by applying sensor 101 to (R)-122 (blue) and (S)-122 (red). CD measurements were taken at 0.15 mM in tetrahydrofuran.

FIG. 85 shows the CD spectra obtained by applying sensor 101 to (R,S)-123 (blue) and (S,R)-123 (red). CD measurements were taken at 0.13 mM in tetrahydrofuran.

FIG. 86 shows the CD spectra obtained by applying sensor 101 to (R,S)-124 (blue) and (S,R)-124 (red). CD measurements were taken at 0.10 mM in tetrahydrofuran.

FIG. 87 shows the CD spectra obtained by applying sensor 101 to (R)-125 (blue) and (S)-125 (red). CD measurements were taken at 0.10 mM in tetrahydrofuran.

FIG. 88 shows the CD spectra obtained by applying sensor 101 to (R)-126 (blue) and (S)-126 (red). CD measurements were taken at 0.09 mM in tetrahydrofuran.

FIG. 89 shows the CD spectra obtained by applying sensor 101 to (R)-127 (blue) and (S)-127 (red). CD measurements were taken at 0.10 mM in tetrahydrofuran.

FIG. 90 shows the amino acid substrate scope. A solution of sensor 101 (1.00 mM) and an amino acid (2.00 mM) in 2.5 mL of acetonitrile:aqueous pH 8.5 borate buffer (4:1 v/v, 10.0 mM) was stirred for 5 minutes. CD analysis was performed after dilution with ACN to the final concentration indicated under each Figure (200-400 μL of the reaction mixture were diluted with 2.0 mL of ACN).

FIG. 91 shows the CD spectra obtained by applying sensor 101 to (R)-128 (blue) and (S)-128 (red). CD measurements were taken at 0.07 mM.

FIG. 92 shows the CD spectra obtained by applying sensor 101 to (R)-129 (blue) and (S)-129 (red). CD measurements were taken at 0.09 mM.

FIG. 93 shows the CD spectra obtained by applying sensor 101 to (R)-130 (blue) and (S)-130 (red). CD measurements were taken at 0.07 mM.

FIG. 94 shows the CD spectra obtained by applying sensor 101 to (R)-131 (blue) and (S)-131 (red). CD measurements were taken at 0.07 mM.

FIG. 95 shows the CD spectra obtained by applying sensor 101 to (R,R)-132 (blue) and (S,S)-132 (red). CD measurements were taken at 0.07 mM.

FIG. 96 shows the CD spectra obtained by applying sensor 101 to (R)-133 (blue) and (S)-133 (red). CD measurements were taken at 0.07 mM.

FIG. 97 shows the CD spectra obtained by applying sensor 101 to (R)-134 (blue) and (S)-134 (red). CD measurements were taken at 0.14 mM.

FIG. 98 shows the CD spectra obtained by applying sensor 101 to (R)-135 (blue) and (S)-135 (red). CD measurements were taken at 0.09 mM.

FIG. 99 shows the CD spectra obtained by applying sensor 101 to (R,S)-136 (blue) and (S,R)-136 (red). CD measurements were taken at 0.07 mM.

FIG. 100 shows the CD spectra obtained by applying sensor 101 to (R)-137 (blue) and (S)-137 (red). CD measurements were taken at 0.07 mM.

FIG. 101 shows the CD spectra obtained by applying sensor 101 to (R)-138 (blue) and (S)-138 (red). CD measurements were taken at 0.09 mM.

FIG. 102 shows the CD spectra obtained by applying sensor 101 to (R)-139 (blue) and (S)-139 (red). CD measurements were taken at 0.07 mM.

FIG. 103 shows the color changes of the quinone sensor 101 upon reaction with phenylethylamine. (S)-1-Phenylethylamine (33.3 mM), Et₃N (33.3 mM) and a quinone sensor 101 (16.7 mM) were mixed in 1.5 mL of THF and the reaction was stirred for 5 min, at which a color change was observed. With the quinone sensor 101, a reduction of the reaction mixture with NaBH₄ (33.3 mM) was investigated by UV, CD and NMR spectroscopy. The reduction results in a color change from dark purple to dark yellow. Colors of solutions of the free sensor 101, the amine addition product and the reduced form.

FIG. 104 shows the color changes of the quinone sensor 102 upon reaction with phenylethylamine. (S)-1-Phenylethylamine (33.3 mM), Et₃N (33.3 mM) and a quinone sensor 102 (16.7 mM) were mixed in 1.5 mL of THF and the reaction was stirred for 5 min, at which a color change was observed. Colors of solutions of the free sensor 102 and the addition product.

FIG. 105 shows the color changes of the quinone sensor 103 upon reaction with phenylethylamine. (S)-1-Phenylethylamine (33.3 mM), Et₃N (33.3 mM) and a quinone sensor 103 (16.7 mM) were mixed in 1.5 mL of THF and the reaction was stirred for 5 min, at which a color change was observed. Colors of solutions of the free sensor 103 and the addition product.

FIG. 106 shows the color changes of the quinone sensor 104 upon reaction with phenylethylamine. (S)-1-Phenylethylamine (33.3 mM), Et₃N (33.3 mM) and a quinone sensor 104 (16.7 mM) were mixed in 1.5 mL of THF and the reaction was stirred for 5 min, at which a color change was observed. Colors of solutions of the free sensor 104 and the addition product.

FIG. 107 shows redox chemistry between the loaded quinone and hydroquinone scaffold.

FIG. 108 shows the UV spectrum of the reaction product of (R)-phenylethylamine and sensor 101. UV measurements were taken at 0.04 mM.

FIG. 109 shows the UV spectrum of the reduced state of the reaction product between (R)-phenylethylamine and sensor 101. UV measurements were taken at 0.04 mM.

FIG. 110 shows the CD spectrum the reaction product of (R)-phenylethylamine and sensor 101. CD measurements were taken at 0.16 mM.

FIG. 111 shows the CD spectrum of the reduced state of the reaction product between (R)-phenylethylamine and sensor 101. CD measurements were taken at 0.16 mM.

FIG. 112 shows the NMR analysis of the quinone and hydroquinone products.

FIG. 113 shows the ¹⁹F NMR of the oxidated and reduced state of the reaction product of (S)-1-phenylethylamine and quinone sensor 101.

FIG. 114 shows the ¹³C NMR analysis of the oxidated and reduced state of the reaction product of (S)-1-phenylethylamine and quinone sensor 101.

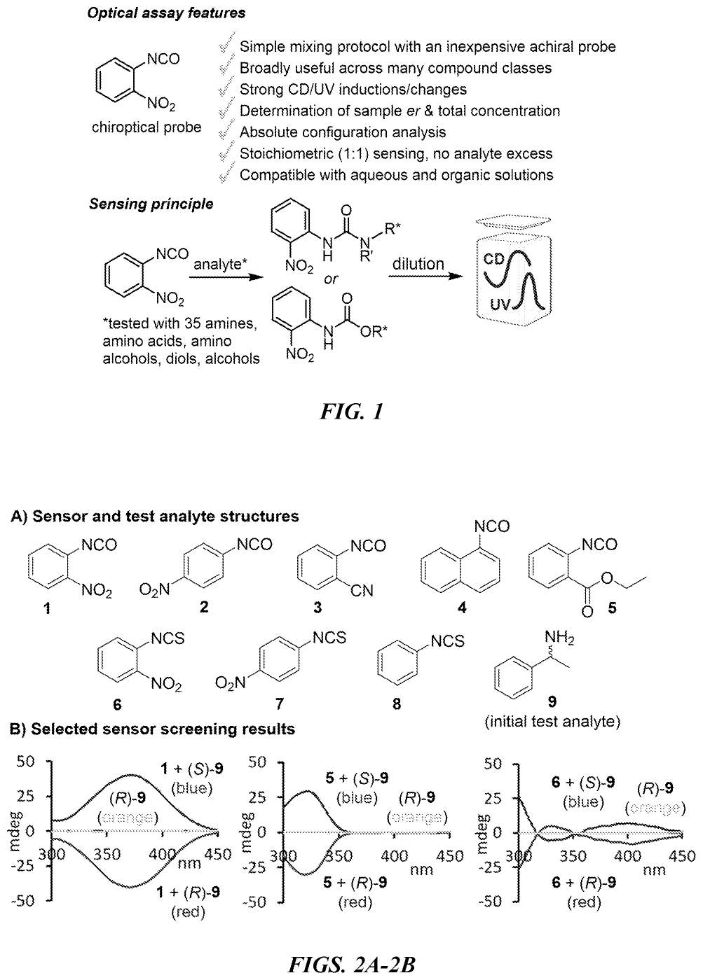

### DETAILED DESCRIPTION

A first aspect of the present application relates to an analytical method which includes providing a sample potentially containing a chiral analyte that can exist in stereoisomeric forms. Furthermore, providing a probe selected from the group consisting of quinones and analogs thereof, (hetero)aryl isocyanates and analogs thereof, and (hetero)aryl isothiocyanates and analogs thereof. The sample is then contacted with the probe under conditions to permit covalent binding of the probe to the analyte, if present in the sample. The method further involves, determining, based on any binding that occurs, absolute configuration of the analyte in the sample and/or the concentration of the analyte in the sample and/or the enantiomeric and/or the diastereomeric composition of the analyte in the sample.

### Probes

The probes of the present application include quinones and analogs thereof, (hetero)aryl isocyanates and analogs thereof, as well as (hetero)aryl isothiocyanates and analogs thereof.

Quinones are a class of organic compounds which possess a fully conjugated cyclic dione structure. Exemplary quinones include, but are not limited to, 1,2-benzoquinone, 1,4-benzoquinone, 1,4-napthoquinone and 9,10-anthraquinone. An analog of a quinone is a quinone in which at least one of the hydrogen atoms has been replaced with a substituent including, but not limited to, a leaving group, a halogen, nitro, cyano, aryl, perfluoroaryl, heteroaryl, cycloalkyl, heterocycloalkyl, alkyl, or perfluoroalkyl.

According to one embodiment, the quinone probe (and analogs thereof) has the structure according to Formula I:

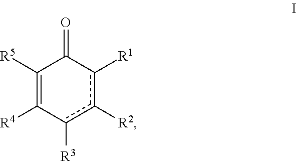

wherein:
R¹ and R³ are independently selected from the group consisting of ═O, X, halogen, —CN, —NO₂, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
R², R⁴, and R⁵ are independently selected from the group consisting of X, halogen, —CN, —C₁-C₆ alkyl, —C₁-C₆ alkoxy, —NO₂, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and
X is a leaving group selected from halogen, —CN, —OR_{b}, —OC(O)R_{b}, —OS(O)₂R_{b}, —S(O)₂—O—R_{b}, —N₂⁺, —N⁺(R_{b})₃, —S⁺(R_{b})₂, and —P⁺(R_{b})₃; wherein each R_{b} is independently selected from the group consisting of -alkyl, —O-alkyl, —N-alkyl, -alkenyl, -alkynyl, -perfluoroalkyl, -perfluoroalkenyl, -perfluoroalkynyl, -aryl, -perfluoroaryl, —O-aryl, —N-aryl, —O-perfluoroaryl, —N-perfluoroaryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
wherein, optionally, R¹ and R², R² and R³, R³ and R⁴, and/or R⁴ and R⁵ are alternatively taken together with the carbon atoms to which they are attached to form a monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{c}, —O—C(O)R_{c}, —NHC(O)R_{c}, —NR_{c}C(O)R_{c}, —NO₂, —CN, -halogen, and —SO₂R_{c}, wherein each Re is independently Ar, alkyl, or CH₂Ar and Ar is an aryl or heteroaryl; with the proviso that one of R¹ or R³ is ═O, and at least one of R¹, R², R³, R⁴, or R⁵ is X.

A (hetero)aryl isocyanate is a (hetero)aryl that possesses at least one isocyanate (—N═C═O) bonded to the (hetero)aromatic ring. An analog of a (hetero)aryl isocyanate is a (hetero)aryl isocyanate in which at least one of the hydrogens on the (hetero)aromatic ring has been replaced with a substituent including, but not limited to halogen, nitro, cyano, aryl, perfluoroaryl, heteroaryl, cycloalkyl, heterocycloalkyl, alkyl, or perfluoroalkyl.

A (hetero)aryl isothiocyanate is an is a (hetero)aryl that possesses at least one isothiocyanate (—N═C═S) bonded to the (hetero)aromatic ring. An analog of a (hetero)aryl isothiocyanate is a (hetero)aryl isothocyanate in which at least one of the hydrogens on the (hetero)aromatic ring has been replaced with a substituent including, but not limited to, halogen, nitro, cyano, aryl, perfluoroaryl, heteroaryl, cycloalkyl, heterocycloalkyl, alkyl, or perfluoroalkyl.

The (hetero)aryl isocyanates and (hetero)aryl isothiocyanates (and analogs thereof) have the structure according to Formula II, IIa, or IIb.

The (hetero)aryl isocyanate or (hetero)aryl isothiocyanate (or an analog thereof) of Formula II has the structure:

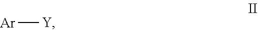

wherein:
Y is —NCO or —NCS; and
Ar is a substituted or unsubstituted aromatic or heteroaromatic chromophore.

The (hetero)aryl isocyanate or (hetero)aryl isothiocyanate (or an analog thereof) of Formula IIa has the structure:

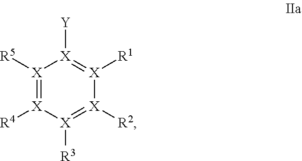

wherein:
Y is —NCO or —NCS;
each X is independently C or N; and
R¹, R², R³, R⁴, and R⁵ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ;
wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and wherein, optionally, R¹ and R², R² and R³, R³ and R⁴, and/or R⁴ and R⁵ are alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

The (hetero)aryl isocyanate or (hetero)aryl isothiocyanate (or an analog thereof) of Formula IIb has the structure:

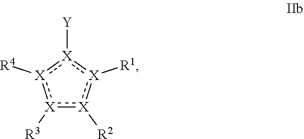

wherein:
Y is —NCO or —NCS;
each X is independently C, N, O or S; and
R¹, R², R³, and R⁴ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
wherein no more than one of R¹, R², R³, or R⁴, is Y; and
wherein, optionally, R¹ and R², R² and R³, and/or R³ and R⁴ is alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

As used herein, the term “alkyl” refers to a straight or branched, saturated aliphatic radical containing one to about twenty (e.g., 1, 2, 3, 4, 5, 6, 7, 8, 9, 10, 11, 12, 13, 14, 15, 16, 17, 18, 19, 20, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms and, unless otherwise indicated, may be optionally substituted. In at least one embodiment, the alkyl is a C₁-C₁₀ alkyl. In at least one embodiment, the alkyl is a C₁-C₆ alkyl. Suitable examples include, without limitation, methyl, ethyl, propyl, isopropyl, butyl, sec-butyl, isobutyl, tert-butyl, 3-pentyl, and the like.

As used herein, the term “alkenyl” refers to a straight or branched aliphatic unsaturated hydrocarbon of formula CₙH₂ₙ having from two to about twenty (e.g., 2, 3, 4, 5, 6, 7, 8, 9, 10, 11, 12, 13, 14, 15, 16, 17, 18, 19, 20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms in the chain and, unless otherwise indicated, may be optionally substituted. Exemplary alkenyls include, without limitation, ethylenyl, propylenyl, n-butylenyl, and i-butylenyl.

As used herein, the term “alkynyl” refers to a straight or branched aliphatic unsaturated hydrocarbon of formula CₙH₂ₙ having from two to about twenty (e.g., 2, 3, 4, 5, 6, 7, 8, 9, 10, 11, 12, 13, 14, 15, 16, 17, 18, 19, 20, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 3-4, 3-5, 3-6, 3-7, 3-8, 3-9, 3-10, 3-11, 3-12, 3-13, 3-14, 3-15, 3-16, 3-17, 3-18, 3-19, 3-20, 4-5, 4-6, 4-7, 4-8, 4-9, 4-10, 4-11, 4-12, 4-13, 4-14, 4-15, 4-16, 4-17, 4-18, 4-19, 4-20, 5-6, 5-7, 5-8, 5-9, 5-10, 5-11, 5-12, 5-13, 5-14, 5-15, 5-16, 5-17, 5-18, 5-19, 5-20, 6-7, 6-8, 6-9, 6-10, 6-11, 6-12, 6-13, 6-14, 6-15, 6-16, 6-17, 6-18, 6-19, 6-20, 7-8, 7-9, 7-10, 7-11, 7-12, 7-13, 7-14, 7-15, 7-16, 7-17, 7-18, 7-19, 7-20, 8-9, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-11, 10-12, 10-13, 10-14, 10-15, 10-16, 10-17, 10-18, 10-19, 10-20, 11-12, 11-13, 11-14, 11-15, 11-16, 11-17, 11-18, 11-19, 11-20, 12-13, 12-14, 12-15, 12-16, 12-17, 12-18, 12-19, 12-20, 13-14, 13-15, 13-16, 13-17, 13-18, 13-19, 13-20, 14-15, 14-16, 14-17, 14-18, 14-19, 14-20, 15-16, 15-17, 15-18, 15-19, 15-20, 16-17, 16-18, 16-19, 16-20, 17-18, 17-19, 17-20, 18-19, 18-20, 19-20) carbon atoms in the chain and, unless otherwise indicated, may be optionally substituted. Exemplary alkynyls include acetylenyl, propynyl, butynyl, 2-butynyl, 3-methylbutynyl, and pentynyl.

As used herein, the term “cycloalkyl” refers to a non-aromatic saturated or unsaturated monocyclic or polycyclic (e.g., bicyclic, tricyclic, tetracyclic) ring system which may contain 3 to 24 (3, 4, 5, 6, 7, 8, 9, 10, 11, 12, 13, 14, 15, 16, 17, 18, 19, 20, 21, 22, 23, 24, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 22-23, 22-24, 23-24) carbon atoms, which may include at least one double bond and, unless otherwise indicated, the ring system may be optionally substituted. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, and syn-bicyclopropane.

As used herein, the term “heterocycloalkyl” refers to a cycloalkyl group as defined above having at least one O, S, and/or N interrupting the carbocyclic ring structure. Examples of heterocycloalkyls include, without limitation, piperidine, piperazine, morpholine, thiomorpholine, pyrrolidine, tetrahydrofuran, pyran, tetrahydropyran, and oxetane. Unless otherwise indicated, the heterocycloalkyl ring system may be optionally substituted.

As used herein, the term “aryl” refers to an aromatic monocyclic or polycyclic (e.g., bicyclic, tricyclic, tetracyclic) ring system from 6 to 24 (6, 7, 8, 9, 10, 11, 12, 13, 14, 15, 16, 17, 18, 19, 20, 21, 22, 23, 24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 22-23, 22-24, 23-24) carbon atoms and, unless otherwise indicated, the ring system may be optionally substituted. Aryl groups of the present technology include, but are not limited to, groups such as phenyl, naphthyl, azulenyl, phenanthrenyl, anthracenyl, fluorenyl, pyrenyl, triphenylenyl, chrysenyl, naphthacenyl, biphenyl, triphenyl, and tetraphenyl. In at least one embodiment, an aryl within the context of the present technology is a 6 or 10 membered ring. In at least one embodiment, each aryl is phenyl or naphthyl.

As used herein, the term “heteroaryl” refers to an aryl group as defined above having at least one O, S, and/or N interrupting the carbocyclic ring structure. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in COMPREHENSIVE HETEROCYCLIC CHEMISTRY: THE STRUCTURE, REACTIONS, SYNTHESIS AND USE OF HETEROCYCLIC COMPOUNDS (Katritzky et al. eds., 1984), which is hereby incorporated by reference in its entirety. Unless otherwise indicated, the heteroaryl ring system may be optionally substituted.

As used herein, the terms “alkoxy” refers to a groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

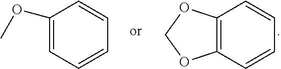

As used herein, the term “aryloxy” refers to —OR, where R is an aryl group.

As used herein, the terms “perfluoroalkyl”, “perfluoroalkenyl”, “perfluoroalkynyl”, and “perfluoroaryl” refer to an alkyl, alkenyl, alkynyl, or aryl group as defined above in which the hydrogen atoms on at least one of the carbon atoms have all been replaced with fluorine atoms.

The term “monocyclic” as used herein indicates a molecular structure having one ring.

The term “polycyclic” as used herein indicates a molecular structure having two or more rings, including, but not limited to, fused, bridged, spiro, or covalently bound rings. In at least one embodiment, the polycyclic ring system is a bicyclic, tricyclic, or tetracyclic ring system. In at least one embodiment, the polycyclic ring system is fused. In at least one embodiment, the polycyclic ring system is a bicyclic ring system such as naphthyl or biphenyl.

As used herein, the term “optionally substituted” indicates that a group may have a substituent at each substitutable atom of the group (including more than one substituent on a single atom), provided that the designated atom's normal valency is not exceeded and the identity of each substituent is independent of the others. “Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” is meant a compound that is sufficiently robust to survive isolation to a useful degree of purity from a reaction mixture, and formulation into an efficacious agent.

As used herein, the term “unsubstituted” means that atoms bear all of the hydrogen atoms dictated by their valency.

As used herein, the term “halogen” includes fluorine, bromine, chlorine, and iodine.

As used herein, “leaving groups” are substituents that are present on the compound that can be displaced. Suitable leaving groups are apparent to a skilled artisan.

As used herein, an “aromatic or heteroaromatic chromophore” refers to an aromatic or heteroaromatic group that produces a signal that can be used for detection through methods such as UV/Vis spectroscopy or fluorescence spectroscopy.

A UV chromophore shows a good absorption behavior in the spectral range of the UV rays or preferably an absorption maximum above 250 nm. The chromophore absorbs the energy of the ultraviolet light and preferably does not change chemically as a result. The energy can be released as heat or phosphorescence/fluorescence. Visible chromophores include compounds with absorption from about 380 nm to 740 nm, which absorb light in the visible spectrum. UV/Vis chromophores have a conjugated pi system, such as those found in aromatic compounds.

A fluorophore refers to a molecule or a functional group in a molecule that absorbs energy of a specific wavelength and re-emits energy at a different wavelength. Fluorescence of organic molecules is closely associated with delocalized electronic structure, such as seen in extended conjugated π systems, which absorb UV or visible light (Fu et al., “Small-Molecule Fluorescent Probes and Their Design,” RSC Adv. 8:29051-61 (2018), which is hereby incorporated by reference in its entirety). Absorbance of light by a conjugated π system result when the energy of incoming UV and/or visible light matches the electronic gap between the bonding to non-bonding electronic orbital levels (π/π*). Id. This allows for the excitation of an electron to a higher energy orbital. Fluorescence is the emission of a photon of energy from the relaxation of the excited electron. Id. Because the excitation and emission wavelengths are different, emission intensity can be measured with little interference from the incoming excitation light. Id. Exemplary fluorophore moieties that may be useful in the present application are disclosed in U.S. Patent Publication No. 2016/0033521 to Higgs, U.S. Pat. No. 7,381,818 to Lokhov et al., and U.S. Pat. No. 6,766,183 to Walsh et al., each of which is hereby incorporated by reference in its entirety.

### Analytes

The analytical methods described herein may be used to evaluate a wide range of chiral analytes. The analyte is one that can exist in stereoisomeric forms. This includes enantiomers, diastereomers, and a combination thereof.

In at least one embodiment, the analyte has low nucleophilicity. Analytes with low nucleophilicity include, for example, alcohols.

In at least one embodiment, when the probe is a quinone or analog thereof, the analyte is selected from primary amines, secondary amines, diamines, amino alcohols, amino acids, amides, and combinations thereof.

In at least one embodiment, when the probe is a (hetero)aryl isocyanate or analog thereof, or a (hetero)aryl isothiocyanate or analog thereof, the analyte is selected from the group consisting of primary amines, secondary amines, diamines, amino alcohols, alcohols, diols, amino acids, thiols, and combinations thereof.

### Reaction Conditions

In at least one embodiment of any analytical method herein, contacting is carried out for about 1 to about 300 minutes (e.g., carried out for a duration range having an upper limit of about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300 minutes, and a lower limit of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, or about 290 minutes, or any combination thereof). In all embodiments, contacting is carried out for a time that is sufficient for the probe to bind to any analyte present in the sample. As will be apparent to the skilled chemist, the speed at which binding takes place will depend on various factors, including the particular probe selected and the analyte, whether a catalyst is present, concentrations, and the temperature.

As will be apparent to the skilled chemist, the analytical methods may be carried out at room temperature, at high temperatures (e.g., about 50° C. to about 100° C., e.g., a temperature range with an upper limit of about 55° C., about 60° C., about 65° C., about 70° C., about 75° C., about 80° C., about 85° C., about 90° C., about 95° C., or about 100° C., and a lower limit of about 50° C., about 55° C., about 60° C., about 65° C., about 70° C., about 75° C., about 80° C., about 85° C., about 90° C., or about 95° C., or any combination thereof), or at low temperatures (e.g., below about 25° C., e.g., below about 25° C., below about 20° C., below about 15° C., below about 10° C., below about 5° C., below about 0° C., below about −5° C., below about −10° C., below about −15° C., below about −20° C., below about −25° C., below about −30° C., below about −35° C., below about −40° C., below about −45° C., below about −50° C., below about −55° C., below about −60° C., below about −65° C., below about −70° C., or below about −75° C., preferably no lower than about −78° C.; e.g., a temperature range with an upper limit of about 25° C., about 20° C., about 15° C., about 10° C., about 5° C., about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., or about −75° C., and a lower limit of about 20° C., about 15° C., about 10° C., about 5° C., about 0° C., about −5° C., about −10° C., about −15° C., about −20° C., about −25° C., about −30° C., about −35° C., about −40° C., about −45° C., about −50° C., about −55° C., about −60° C., about −65° C., about −70° C., about −75° C., or about −78° C., or any combination thereof). Furthermore, the analytical methods may be carried out under ambient conditions (e.g., 23±3° C. and 38±5% relative humidity).

For example, the temperature could be increased to speed up the binding reaction. Some analyte-probe combinations may have side reactions at certain temperatures; the temperature could be decreased to prevent such side reactions.

The analytical methods could also optionally be carried out in the presence of a base. The use of a base may be helpful when the analyte is an acid (e.g., a carboxylic acid) or when an acid may be generated in situ. Adding an equivalent of base could also be helpful to avoid side reactions. Suitable bases include both organic and inorganic bases (or mixtures thereof). Exemplary bases include, but are not limited to: alkoxides such as sodium tert-butoxide; alkali metal amides such as sodium amide, lithium diisopropylamide, and alkali metal bis(trialkylsilyl)amide, e.g., such as lithium bis(trimethylsilyl)amide (LiHMDS) or sodium bis(trimethylsilyl)amide (NaHIMDS); tertiary amines (e.g. triethylamine, trimethylamine, 4-(dimethylamino)pyridine (DMAP), 1,5-diazabicycl[4.3.0]non-5-ene (DBN), 1,5-diazabicyclo[5.4.0]undec-5-ene (DBU); alkali or alkaline earth carbonate, bicarbonate or hydroxide (e.g. sodium, magnesium, calcium, barium, potassium carbonate, phosphate, hydroxide and bicarbonate); and ammonium hydroxides, e.g. tetrabutylammonium hydroxide (TBAOH).

The analytical methods could also optionally be carried out in the presence of a buffer. Exemplary buffers include, but are not limited to, borate, phosphate, carbonate, Trizma, and Hepes buffers between pH 2-12.

In certain embodiments, the contacting step is carried out in a solvent selected from aqueous solvents, protic solvents, aprotic solvents, and any combination thereof. Exemplary solvents include, but are not limited to, chloroform, dichloromethane, acetonitrile, toluene, tetrahydrofuran, methanol, ethanol, isopropanol, water, dimethyl sulfoxide (DMSO), dimethylformamide (DMF), pentane, pentane isomers, hexane, hexane isomers, ether, dichloroethane, acetone, ethyl acetate, butanone, and mixtures of any combination thereof.

In certain embodiments, the analytical methods are carried out under aerobic conditions (e.g., under air or in an aqueous environment).

### Analysis

In the analytical methods described herein, the probe is reacted with the analyte to form probe-analyte complexes through a covalent bond between the probe and the analyte. The probe-analyte complexes generate a chiroptical signal that can be used to determine the enantiomeric/diastereomeric composition and/or absolute configuration of the analyte.

The term “enantiomeric composition” refers to the enantiomeric ratio and/or enantiomeric excess of an analyte. The enantiomeric ratio (er) is the ratio of the percentage of one analyte enantiomer in a mixture to that of the other enantiomer. The enantiomeric excess (ee) is the difference between the percentage of one analyte enantiomer and the percentage of the other analyte enantiomer. For example, a sample which contains 75% L-analyte and 25% D-analyte will have an enantiomeric excess of 50% of L-analyte and an enantiomeric ratio (D:L) of 25:75.

The term “diastereomeric composition” refers to the diastereomeric ratio and/or diastereomeric excess of an analyte. The diastereomeric ratio is the ratio of the percentage of one analyte diastereomer in a mixture to that of the other diastereomer. The diastereomeric excess is the difference between the percentage of one analyte diastereomer and the percentage of the other analyte diastereomer. For example, a sample which contains 75% R,S-analyte and 25% S,S-analyte will have a diastereomeric excess of 50% of R,S-analyte and a diastereomeric ratio (S,S:R,S) of 25:75.

The enantiomeric/diastereomeric composition of the analyte can be determined by correlating the chiroptical signal of the probe-analyte complexes that form to the enantiomeric/diastereomeric composition of the analyte. The absolute configuration of the analyte can also be assigned from the chiroptical signal of the probe-analyte complexes that form. The configuration assignment can be based on the sense of chirality induction with a reference or by analogy. The chiroptical signal of the probe-analyte complexes can be measured using standard techniques, which will be apparent to the skilled artisan. Such techniques include circular dichroism spectroscopy (e.g., STEREOCHEMISTRY OF ORGANIC COMPOUNDS 1003-07 (E. L. Eliel & S. H. Wilen eds., 1994); DYNAMIC STEREOCHEMISTRY OF CHIRAL COMPOUNDS 140-43 (Christian Wolf ed., 2008), each of which is hereby incorporated by reference in its entirety), optical rotatory dispersion (e.g., STEREOCHEMISTRY OF ORGANIC COMPOUNDS 999-1003 (E. L. Eliel & S. H. Wilen eds., 1994), which is hereby incorporated by reference in its entirety), and polarimetry (e.g., STEREOCHEMISTRY OF ORGANIC COMPOUNDS 217-21, 1071-80 (E. L. Eliel & S. H. Wilen eds., 1994); DYNAMIC STEREOCHEMISTRY OF CHIRAL COMPOUNDS 140-43 (Christian Wolf ed., 2008), each of which is hereby incorporated by reference in its entirety). By way of example, stereomerically pure samples of each isomer of an analyte of interest can be mixed with the particular probe to generate standard samples, and their optical spectra obtained. The chiroptical signal of the probe-analyte complexes in the test sample can be measured by generating an optical spectrum of the test sample. The enantiomeric composition and/or the diastereomeric composition and/or absolute configuration of the analyte originally present in the sample can then be determined by comparing the optical spectrum of the test sample to that of the standard sample(s).

In the analytical methods described herein, the concentration of the analyte can be determined by correlating an optical spectroscopic signal of the probe-analyte complexes that form to that of standard samples. The optical spectroscopic signal can be measured using standard techniques, which will be apparent to the skilled artisan. Such techniques include, but are not limited to, UV spectroscopy (PRINCIPLES OF INSTRUMENTAL ANALYSIS 342-47 (Douglas A. Skoog et al. eds., 5^{th} ed. 1998), which is hereby incorporated by reference in its entirety), fluorescence spectroscopy, and other spectroscopic techniques. By way of example, serial titrations of the analyte of interest can be mixed with the particular probe to generate standard samples and their spectra (e.g., UV, fluorescence) obtained. The spectroscopic signal (e.g., UV, fluorescence) of the probe-analyte complexes can be measured by generating a spectrum (e.g., UV, fluorescence) of the test sample. The total concentration of the analyte originally present in the test sample can then be determined by comparing the spectrum of the test sample to the calibration curve obtained with the standard samples. As will be apparent to the skilled artisan, if the stereoisomeric excess of the analyte is also determined, the concentration of individual isomers originally present in the test sample can be determined by comparing the stereoisomeric excess to the total analyte concentration.

The analytical methods of the present application provide, among other things, rapid and convenient tools for simultaneously determining the concentration as well as the enantiomeric composition and/or the diastereomeric composition and/or absolute configuration of chiral analytes. These analytical methods may be particularly useful, for example, for evaluating high-throughput reactions whose desired product is chiral. For example, the present methods can be used to determine the enantiomeric/diastereomeric composition of the desired product, thus indicating the stereoselectivity of the reaction. Similarly, the present methods can be used to determine the concentration of the total product and/or the desired isomer, thus indicating the overall or individual yield of the reaction.

Furthermore, the analytical methods of the present application allow for the determination of two or three of the absolute configuration, the concentration, the enantiomeric composition, and the diastereomeric composition of the analyte. It is also possible to use the analytical method to determine the absolute configuration, the concentration, the enantiomeric composition, and the diastereomeric composition of the analyte.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

### EXAMPLES

The present technology may be further illustrated by reference to the following examples, which are intended to exemplify the practice of embodiments of the disclosure but are by no means intended to limit the scope thereof.

### Example 1—Chirality Sensing of Amines and Amino Acids with Achiral Iso(Thio)Cyanates

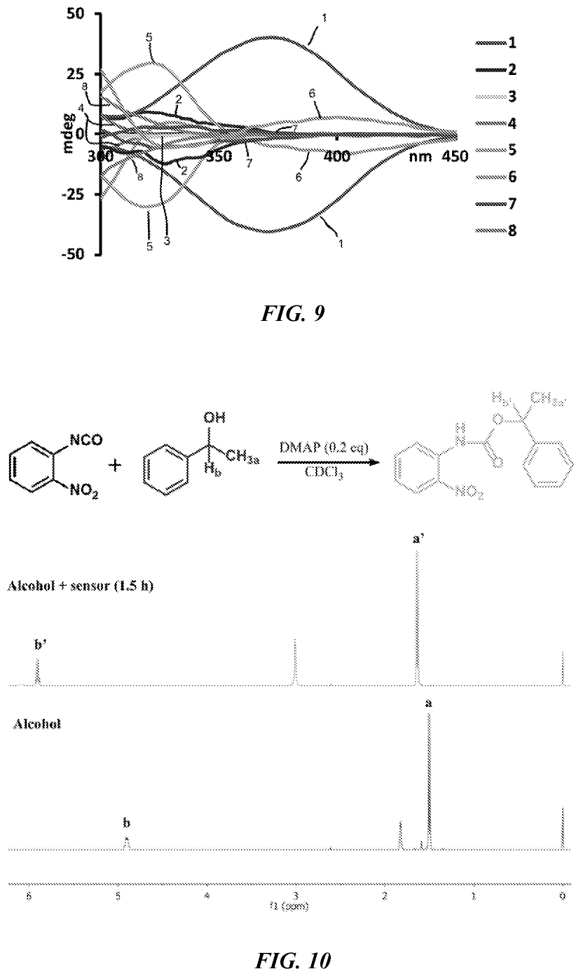

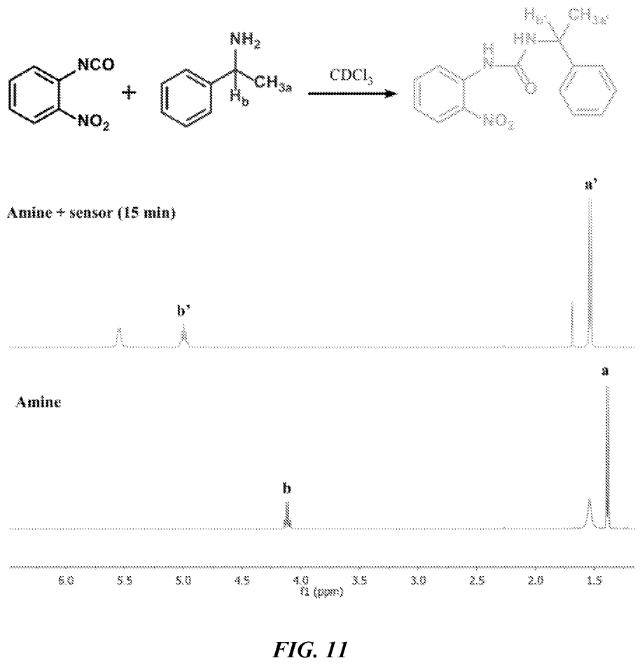

Aryliso(thio)cyanates 1-8 were initially screened for their ability to react with the enantiomers of 1-phenylethylamine, 9, toward a CD-active (thio)urea product. All probes carry a chromophore in close proximity of the analyte binding unit, which was deemed essential to generate a distinct CD signal suitable for quantitative analysis (FIGS. 2A-2B; 8). Initial optimization was carried out with 1-phenylethylamine 9 (82.5 mM) and an iso(thio)cyanate sensor 1-8 (99.0 mM), which were mixed in 1.0 mL of CHCl₃ and the reaction was stirred overnight followed by CD analysis in 2.0 mL of ACN (0.10-1.24 mM). The urea derived from sensor 1 yielded the strongest red-shifted CD signal (FIG. 9). The urea formation occurred quantitatively and without by-product formation within 15 minutes, which was verified by NMR analysis (FIG. 11). CD signals were observed with high amplitudes above 350 nm using 2-nitrophenyl-isocyanate, 1. Strong CD signal induction at long wavelengths was generally advantageous to eliminate possible interferences from chiral impurities that may produce CD signals below 300 nm. The other probes produced blue-shifted CD signals with the exception of the thioisocyanate analogue 6, which showed a relatively weak maximum at 400 nm (FIG. 2B). As expected, the free (R)-enantiomer of amine 9 showed no CD signal in the same region.

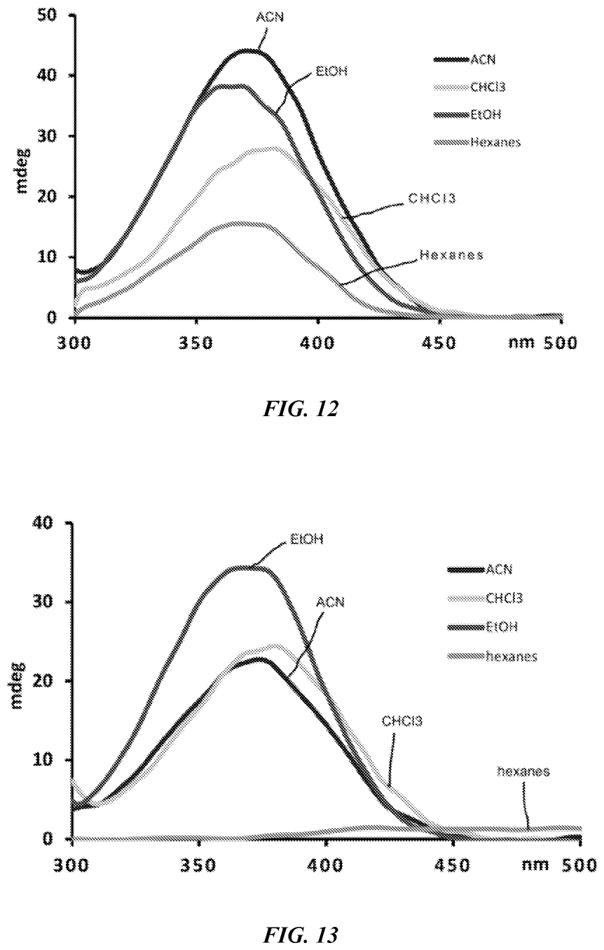

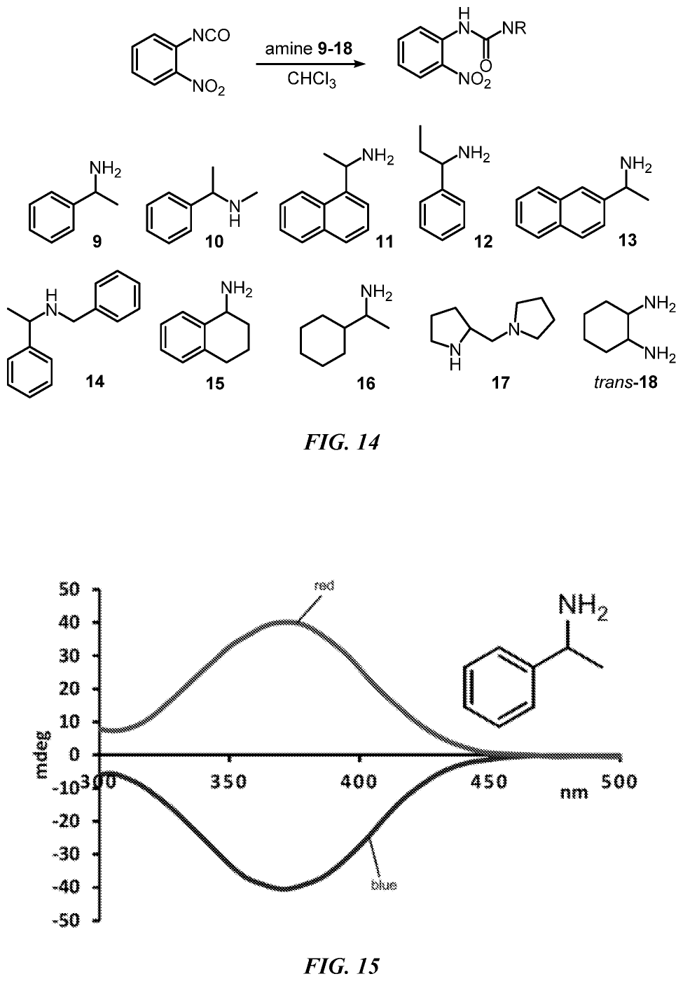

The urea formation from 1 and 9 can be conducted in a variety of organic solvents ranging from hexane to ethanol and the sensing experiments do not require any precautions. The urea products are stable and the sensing mixtures are easy to handle. The best results were obtained when the reactions were performed in chloroform and diluted with acetonitrile for CD analysis which were conducted without delay. These solvent screenings were initially carried out using (S)-phenylethylamine (82.5 mM) 9 and sensor 1 (99.0 mM), which involved mixing of the sensor and analyte for 15 minutes in either 1.0 mL of CHCl₃ or 1.0 mL of ACN, which was then diluted to a final volume of 2.0 mL with ACN, CHCl₃, EtOH, or hexane (0.41 mM) for CD analysis. The reaction run in CHCl₃ followed by dilution with ACN yielded the strongest CD signal (compare FIGS. 12-13). All subsequent reactions were thus run in CHCl₃ and diluted with ACN for CD analysis. This simple mix-dilute-measure protocol was essentially used for all applications with only minor modifications that were necessary to dissolve amino acids or to extend the sensing scope to alcohols and amino alcohols (see Example 2, infra)

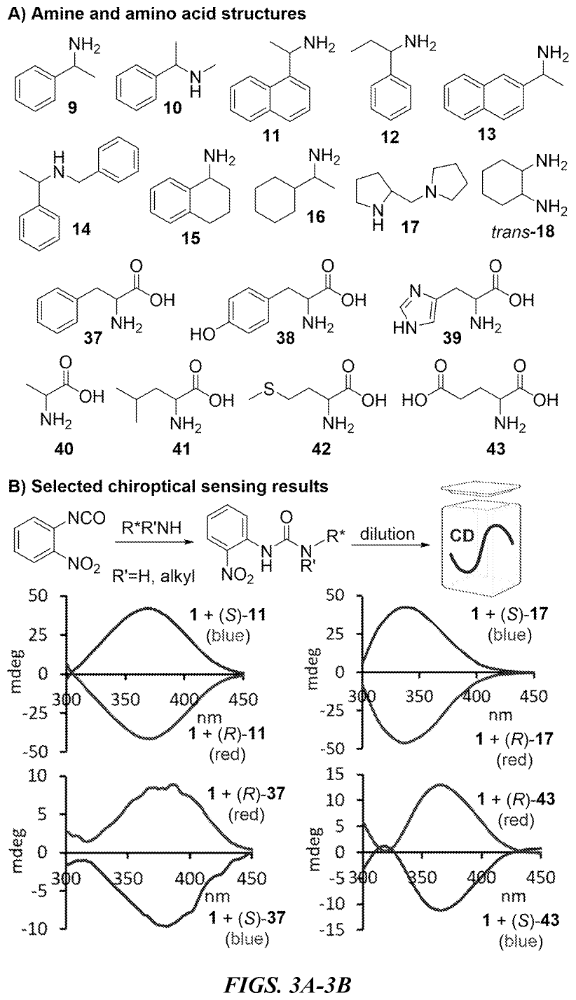

The CD sensing utility of 1 was investigated with a structurally diverse group of aromatic and aliphatic amines 9-18 in this straightforward protocol (FIGS. 3A-B; 14).

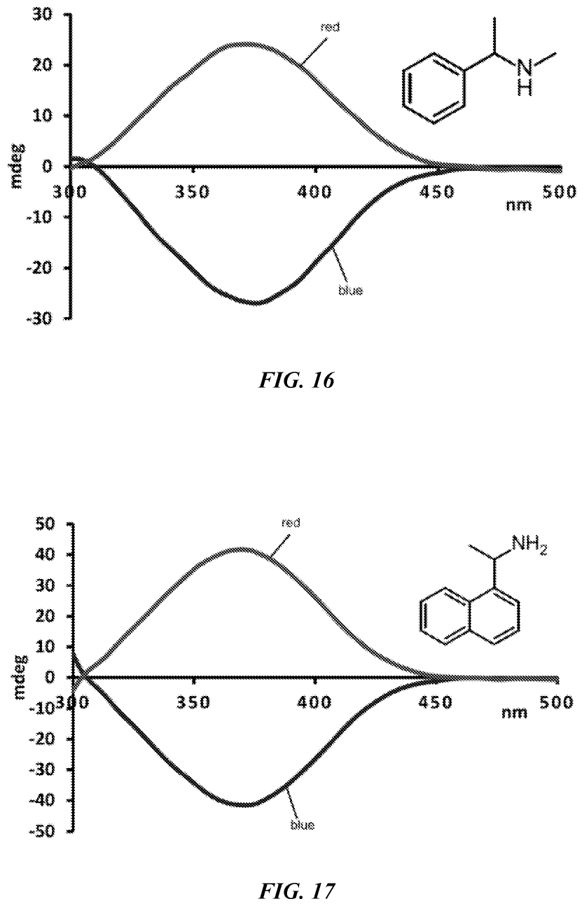

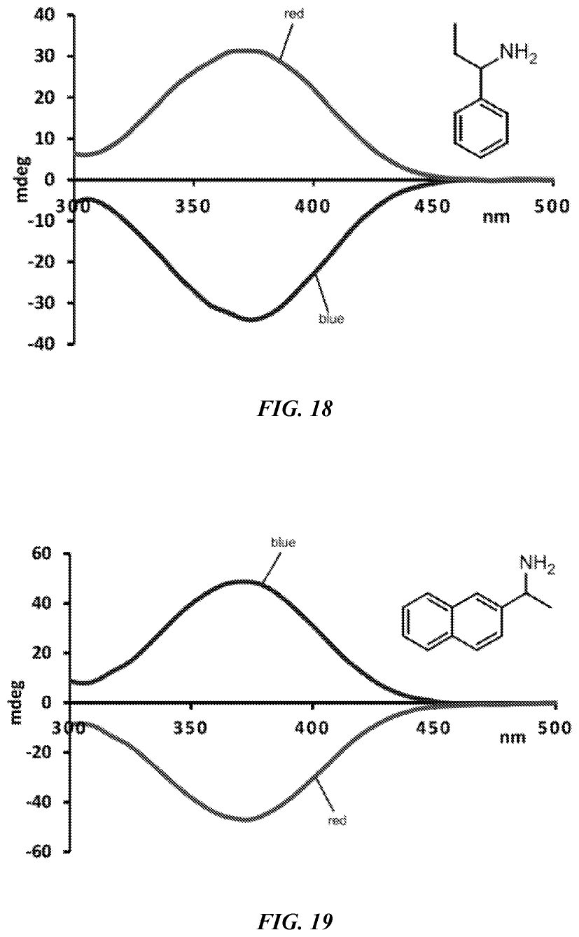

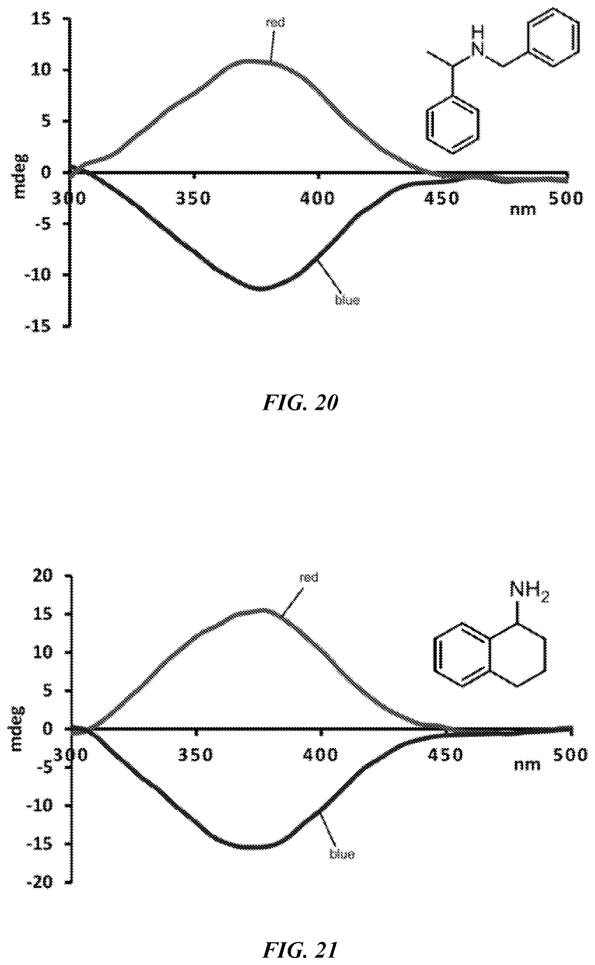

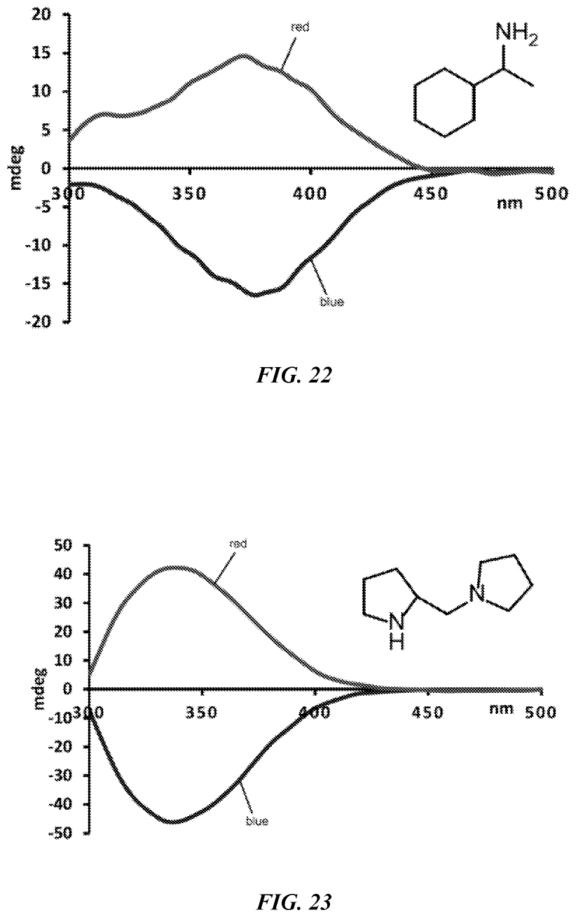

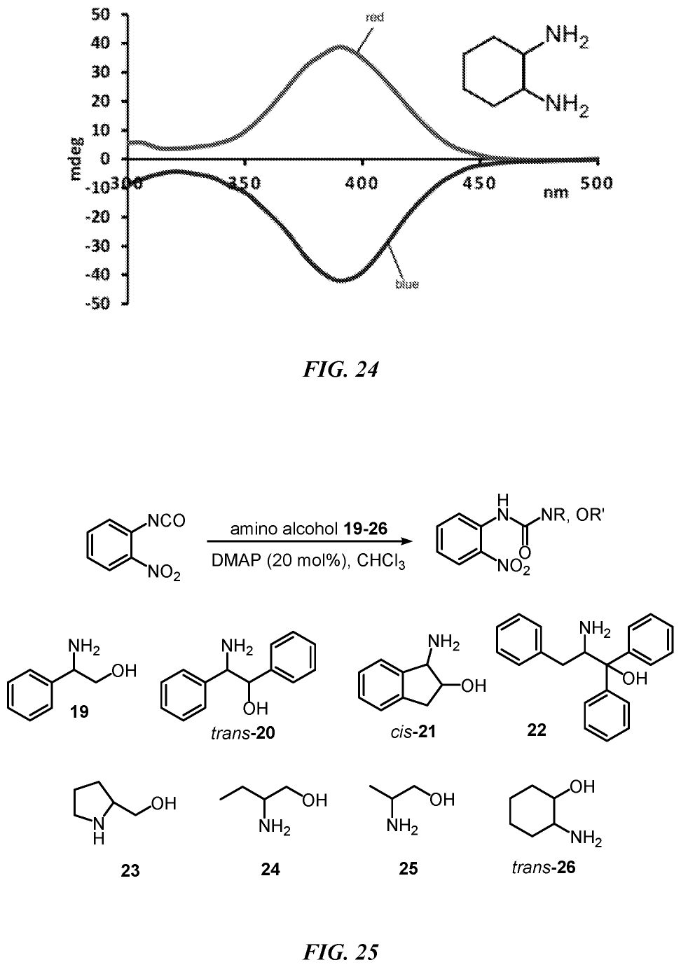

A solution of chiral amines 9-17 (45-85 mM) and sensor 1 (1.2 equivalents) in 1.0 mL of chloroform was stirred for 15 minutes. For chiral diamine 18, 2.4 equivalents of the sensor were used. CD analysis was performed after dilution with ACN to the final concentration indicated in the figure descriptions (20.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation. All analytes gave quantifiable CD signals as shown exemplarily for 11 and 17, a secondary amine that cannot be analyzed with optical Schiff base sensors (FIG. 3B). Additional CD signal results are illustrated in FIGS. 15-24.

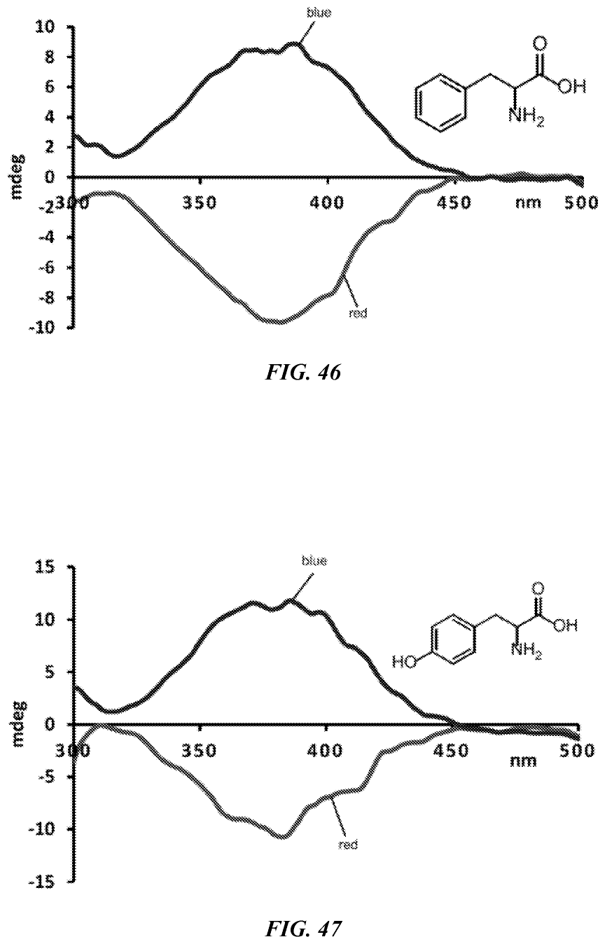

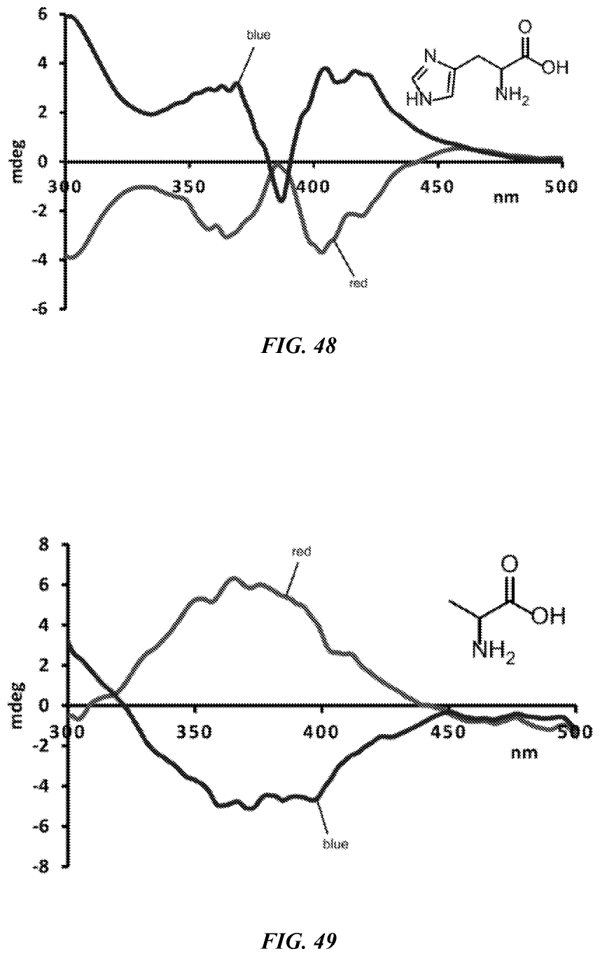

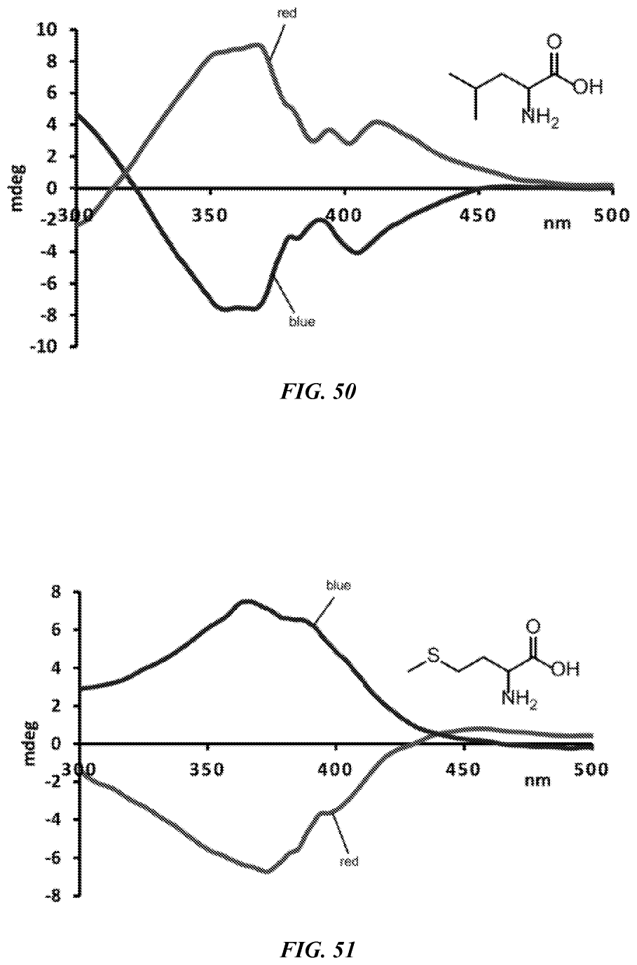

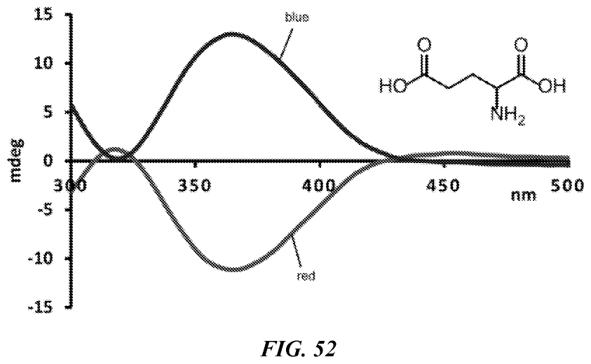

The CD sensing utility of 1 was also investigated with various amino acids 37-43 in this straightforward protocol (FIGS. 3A-B; 45). A solution of amino acids 37-43 (9.0 mM), sensor 1 (10.8 or 21.6 mM), and Na₂CO₃ (18.0 mM) in 1.0 mL of a 4:1 ACN:water mixture was stirred overnight. For amino acids 37 and 40-43, 1.2 equivalents of the sensor (10.8 mM) were used. For amino acids 38 and 39, 2.4 equivalents of the sensor (21.6 mM) were used. For tyrosine (38), DMAP (0.2 equivalents) was used as a catalyst. CD analysis was performed after dilution with ACN to the final concentration (50.0-150.0 μL of the reaction mixture added to 2.0 mL ACN) indicated in the figures. The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation. Aqueous solutions were tolerated, which enabled the use of 1 for chiroptical sensing of amino acids including alanine (40) and glutamic acid (43, FIG. 3B). Additional CD signal results are illustrated in FIGS. 46-52.

### Example 2—Chirality Sensing of Alcohols and Amino Alcohols with Achiral Isocyanate Sensor

The success with amine and amino acid chirality sensing via irreversible urea formation and direct CD analysis encouraged the evaluation of alcohols and amino alcohols as analytes. In particular, alcohols remain among the most challenging sensing targets to date. This can be attributed to their low nucleophilicity, which greatly complicates the development of optical chirality sensing assays based on well-defined stoichiometric molecular recognition processes or the formation of supramolecular assemblies that should preferentially occur under mild conditions and within a few hours. The daunting task of alcohol chirality sensing was envisioned to be feasible through covalent trapping as carbamate derivatives of sensor 1.

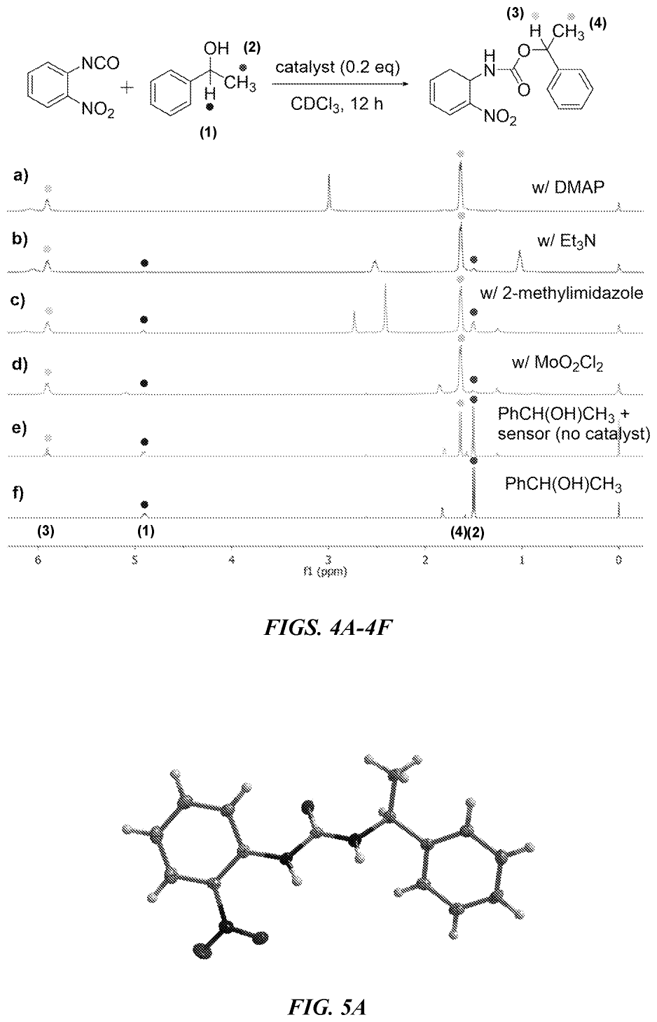

Initial tests with phenylethanol were monitored by ¹H NMR, which showed that the carbamate formation with 1 was slow at room temperature (FIG. 10). Therefore, several catalysts were screened for compatibility with the optical measurements to avoid any need to work-up the reaction mixtures prior to the CD analysis. See FIGS. 4A-4F.

The quantitative carbamate conversion was accomplished in the presence of 20 mol % of DMAP. The reaction does not generate by-products and was complete within 1.5 hours at room temperature. This was sufficiently time-efficient even for high-throughput purposes, because one could run and analyze hundreds of samples in parallel using multiwell-plate CD readers. However, one could further accelerate the carbamate formation by gentle heating, if desired. During the course of this analysis, single crystals were grown by slow evaporation of dichloromethane:hexane solutions of the urea and carbamate formed in the reaction of 1 with an amine and an alcohol, respectively. The crystallographic structure elucidation further corroborates the results and conclusions of this NMR reaction analysis. See FIG. 5.

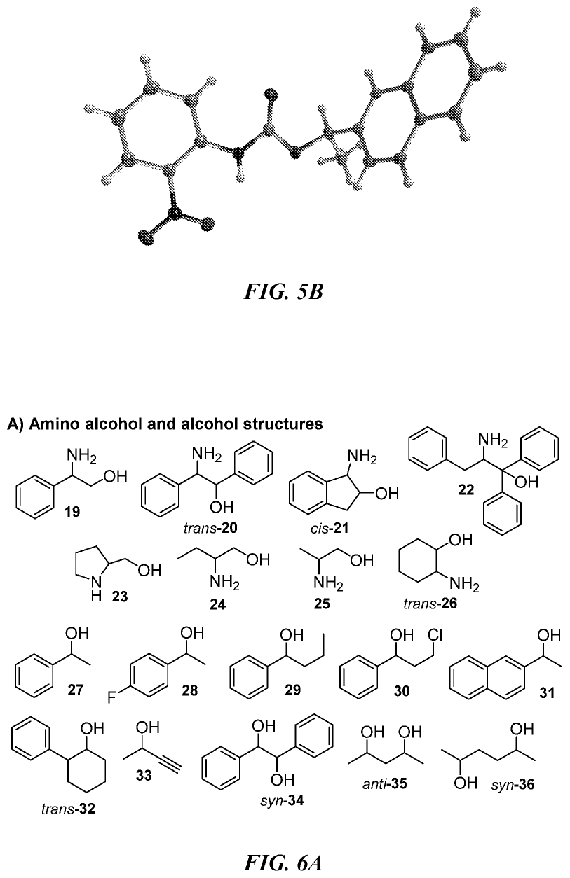

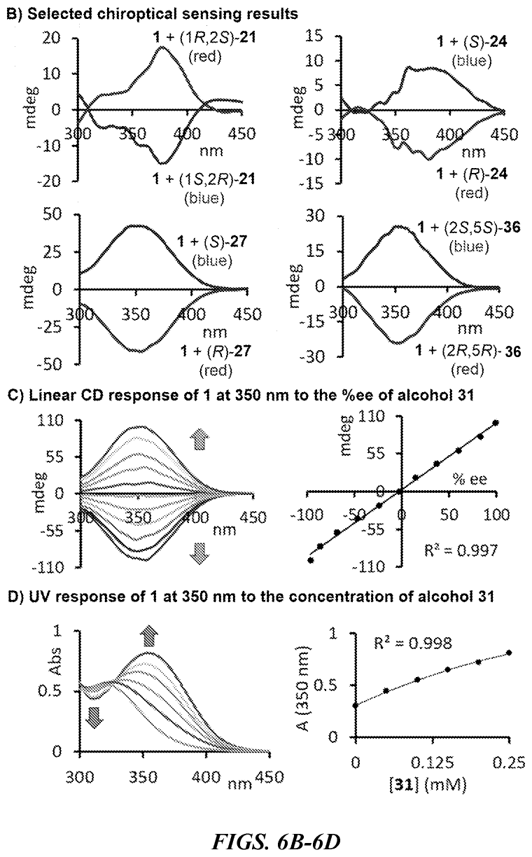

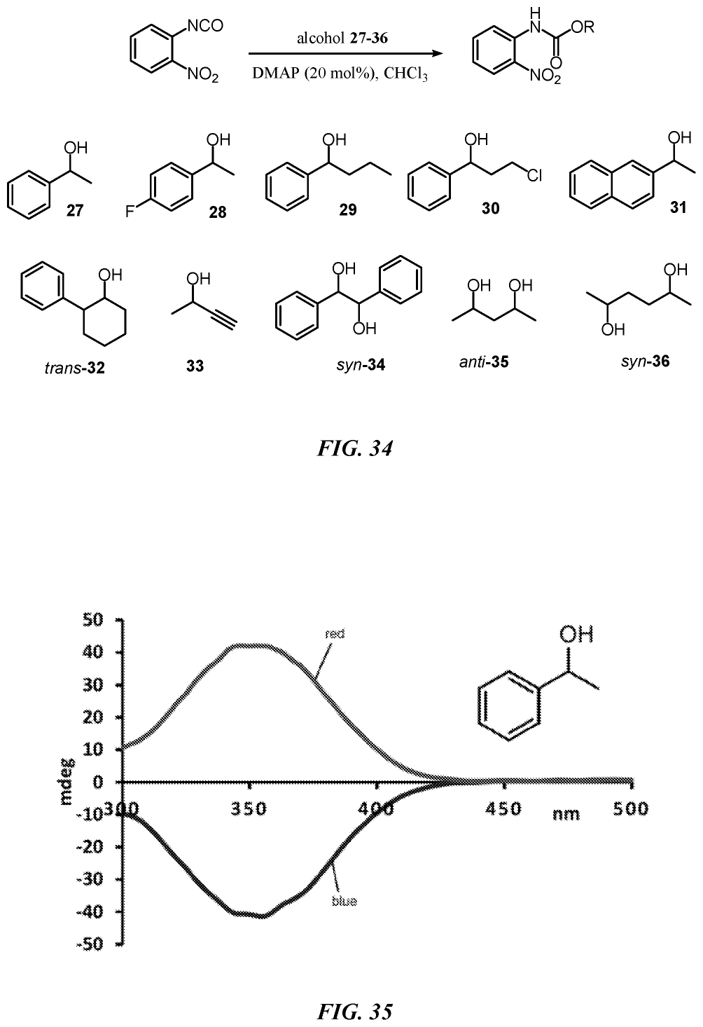

With a slightly revised sensing protocol in hand, the application spectrum examination was continued for sensor 1 by testing a broad variety of amino alcohols, alcohols and diols 19-36 (FIGS. 6A, 25, 34). Some of these target structures carry phenyl rings that might be beneficial for the generation of strong, red-shifted CD signals once the carbamate was formed, but also included were purely aliphatic compounds such as 23-26, 33, 35 and 36.

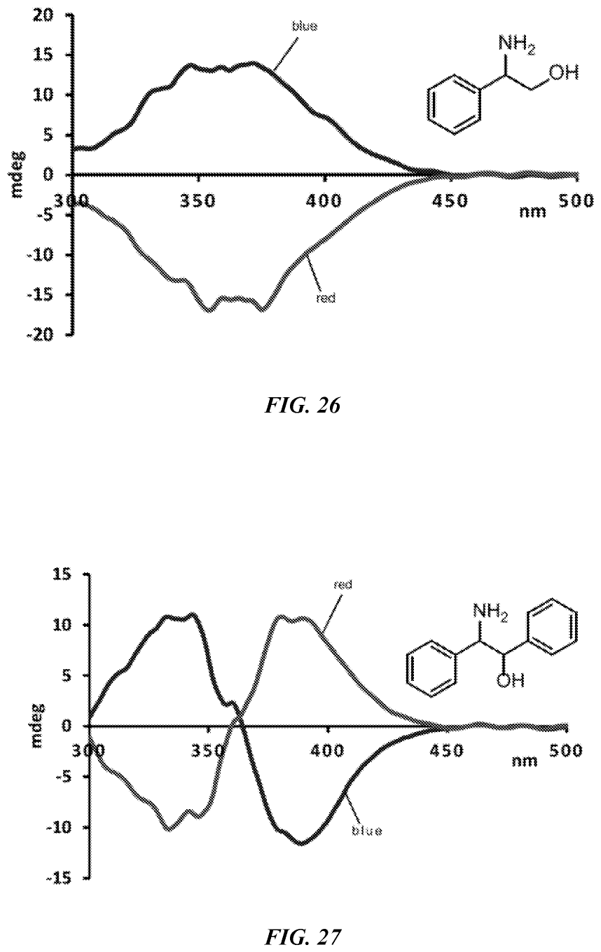

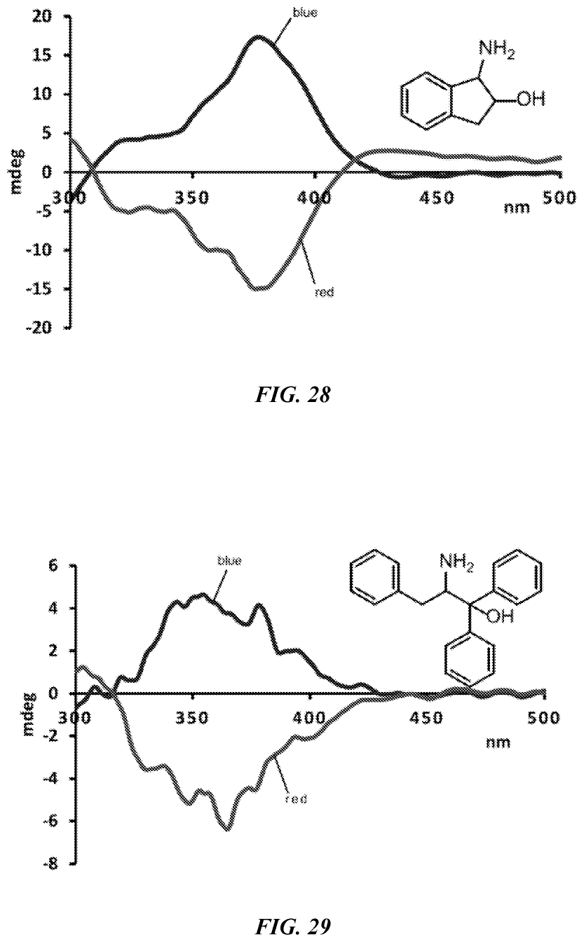

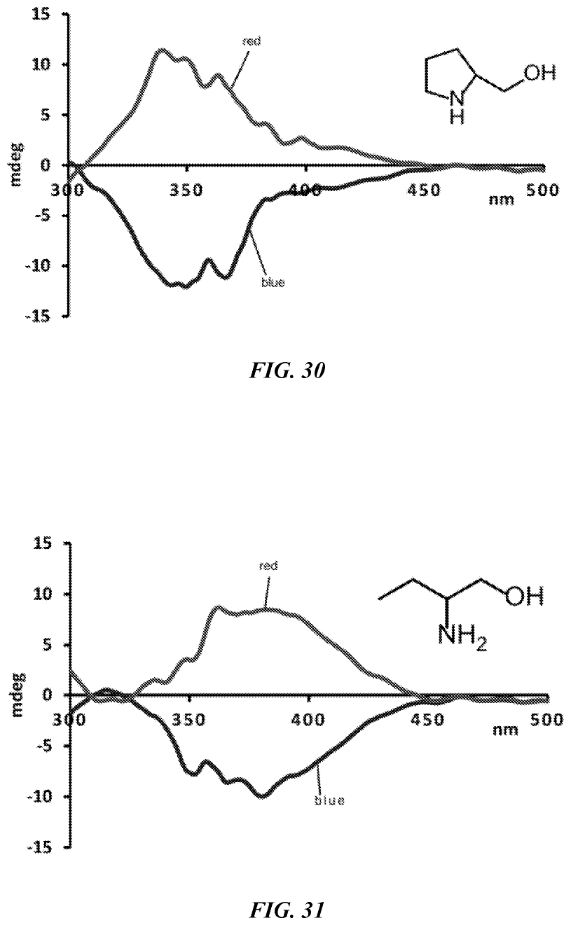

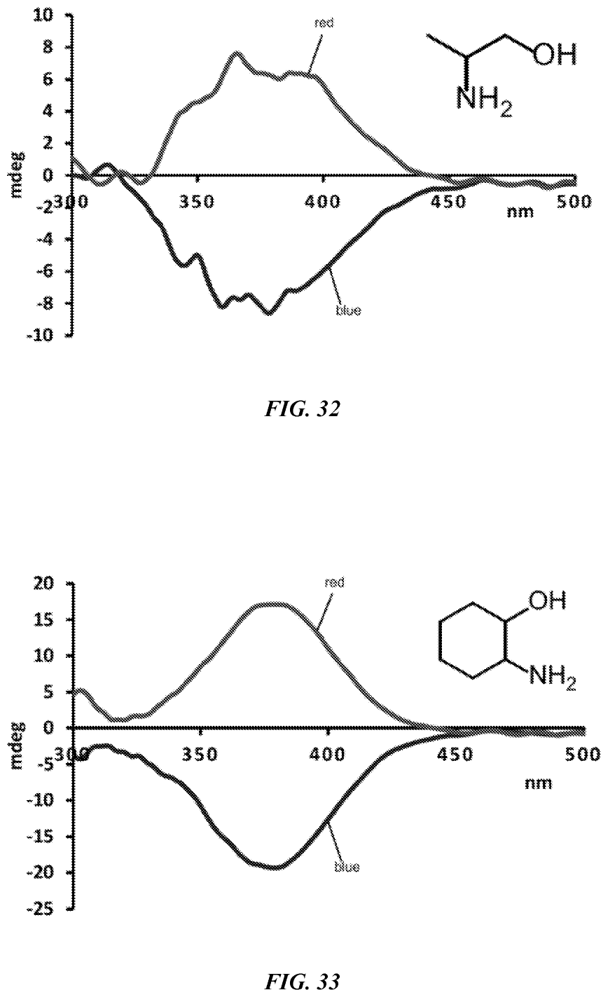

In a glovebox, a solution of chiral amino alcohols 19-26 (27-100 mM), sensor 1 (2.4 equivalents), and DMAP (0.2 equivalents) in 2.0 mL of chloroform was stirred overnight. CD analysis was performed after dilution with ACN to the final concentration indicated in the figure descriptions (15.0-40.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation. CD signal results are illustrated in FIGS. 26-33.

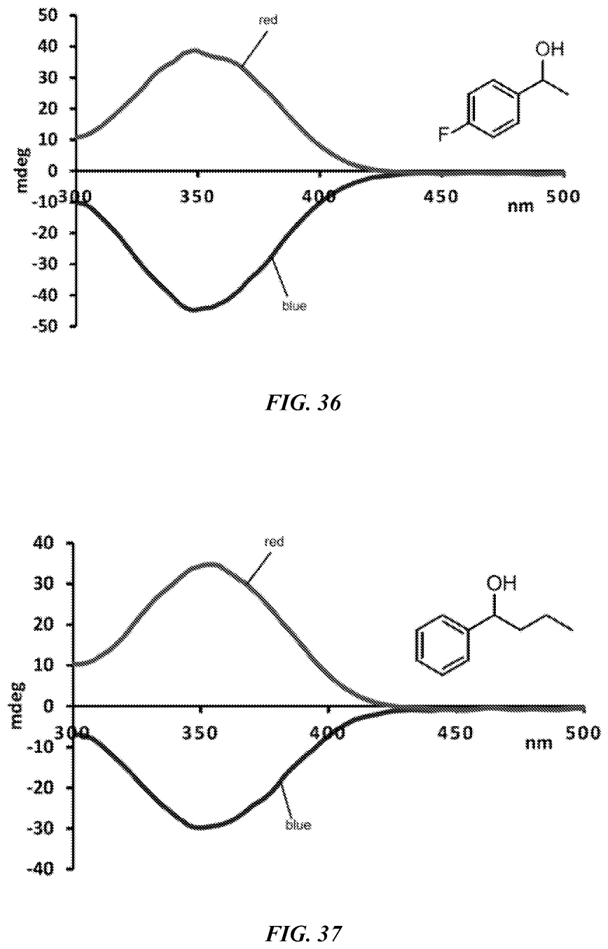

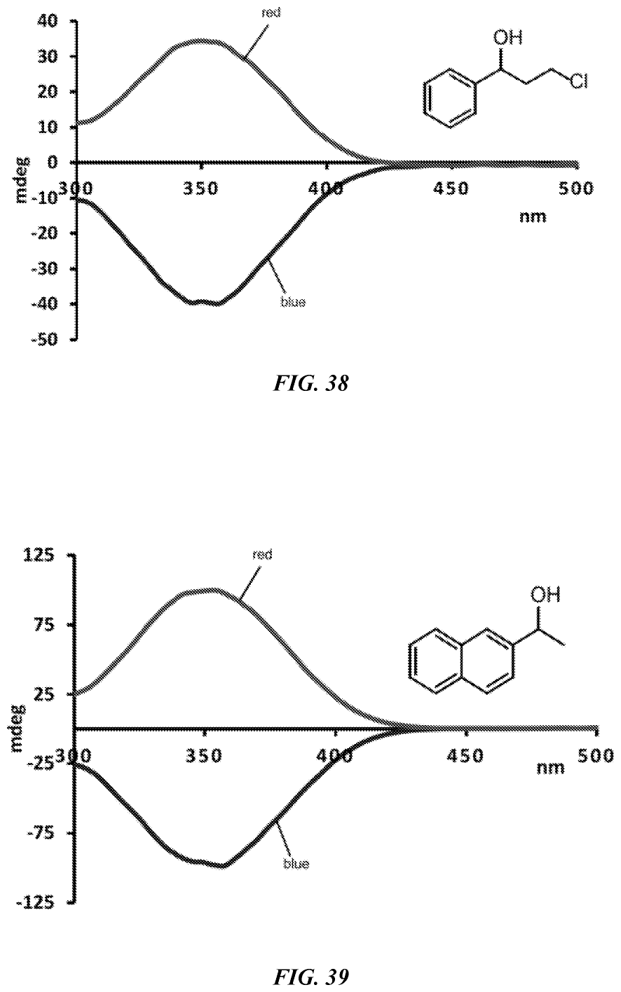

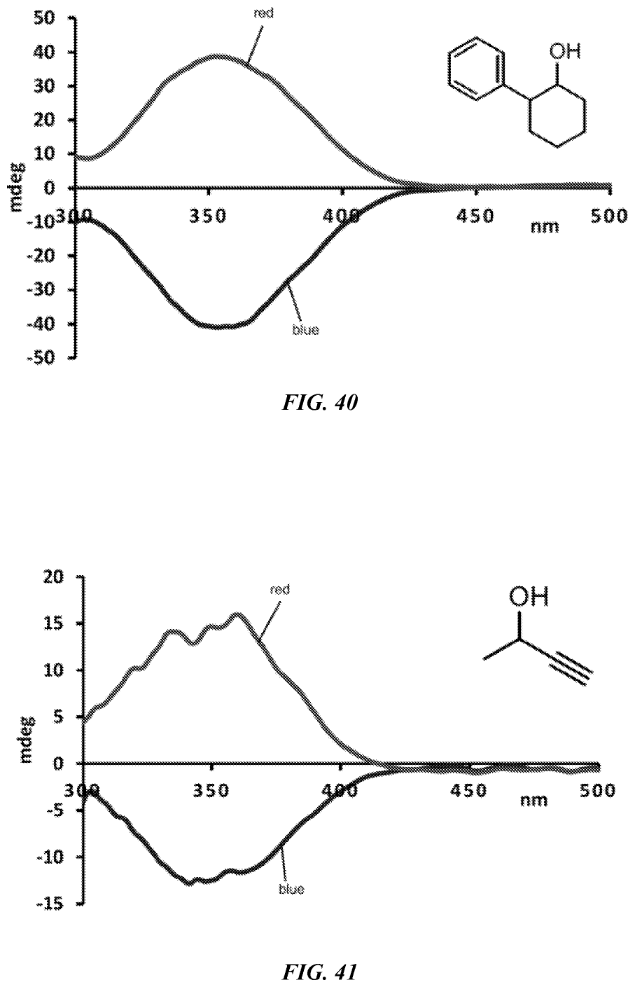

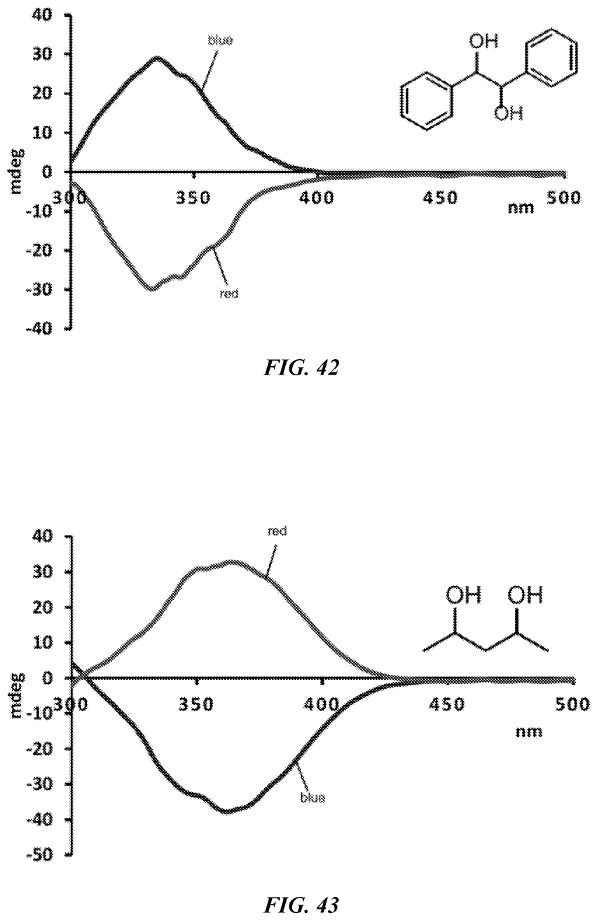

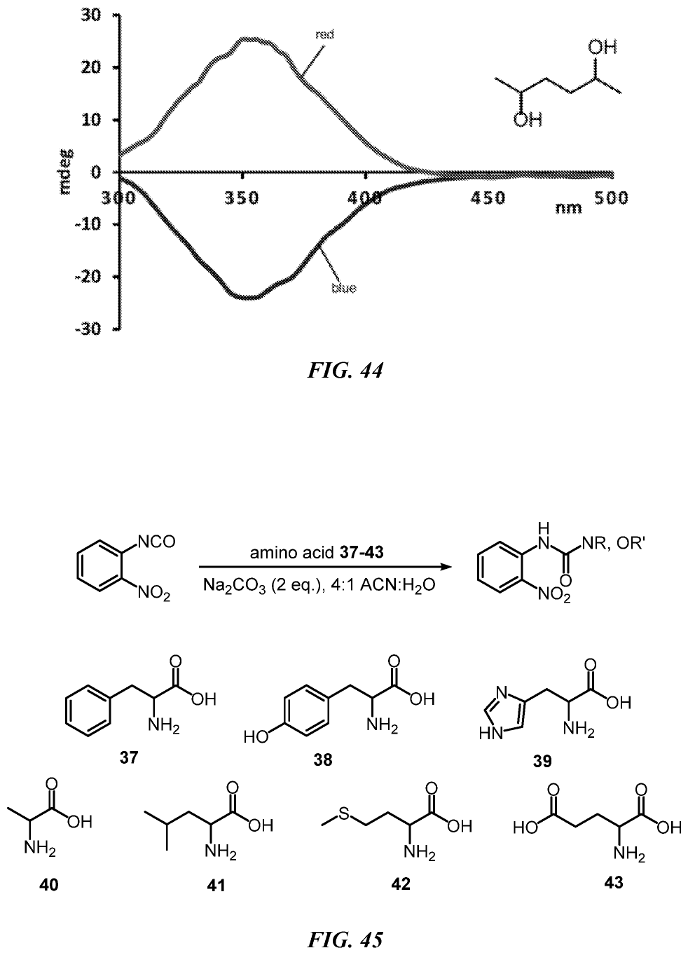

In a glovebox, a solution of chiral alcohols 27-36 (70-130 mM), sensor 1, and DMAP (0.2 equivalents) in 1.0 mL of chloroform was stirred overnight. For chiral alcohols 27-33, 1.2 equivalents of the sensor were used; for diols 34-36, 2.4 equivalents of the sensor were used. CD analysis was performed after dilution with ACN to the final concentration indicated in the figure descriptions (5.0-20.0 μL of the reaction mixture added to 2.0 mL ACN). The CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation. CD signal results are illustrated in FIGS. 35-44

Selected CD spectra for sensor 1 with 21, 24, 27, and 36 are shown in FIG. 6B. Again, the distinct CD signals were observed in all cases, which underscores the practicality and the broad utility of this chirality sensing assay just mix the sensor with the target compound and take chiroptical measurements upon dilution without any purification and chromatographic separation. Based on the well-defined sensing stoichiometry, a linear correlation was observed between the CD response measured at 350 nm and the chiral composition of the alcohol 31, which was in perfect agreement with the proposed carbamate formation. See FIG. 6C.

### Example 3—Chirality Sensing for Combined er and Concentration Analysis

The preceding results demonstrate that this chiroptical assay works with, but was not limited to, 35 amines, amino alcohols, amino acids, diols and alcohols, which stands out among previously reported sensing methods. The generality of its usefulness prompted evaluation of the possibility of combined er and concentration analysis. As shown in the preceding examples, the CD response generated by chromophoric probe 1 upon binding of a chiral compound changes linearly with the enantiomeric composition of the sample, and this simplifies the quantitative analysis tasks. The formation of a carbamate or urea product was suspected to be derived from 1 and either an alcohol or amine would also yield a characteristic UV change. Because the use of an achiral sensor-in contrast to a chiral derivatizing agent typically employed in NMR or HPLC analysis of chiral compounds-enantiomeric rather than diastereomeric products are formed, and the corresponding UV signals can therefore be directly correlated to the total concentration of the chiral analyte, irrespective of the sample er.

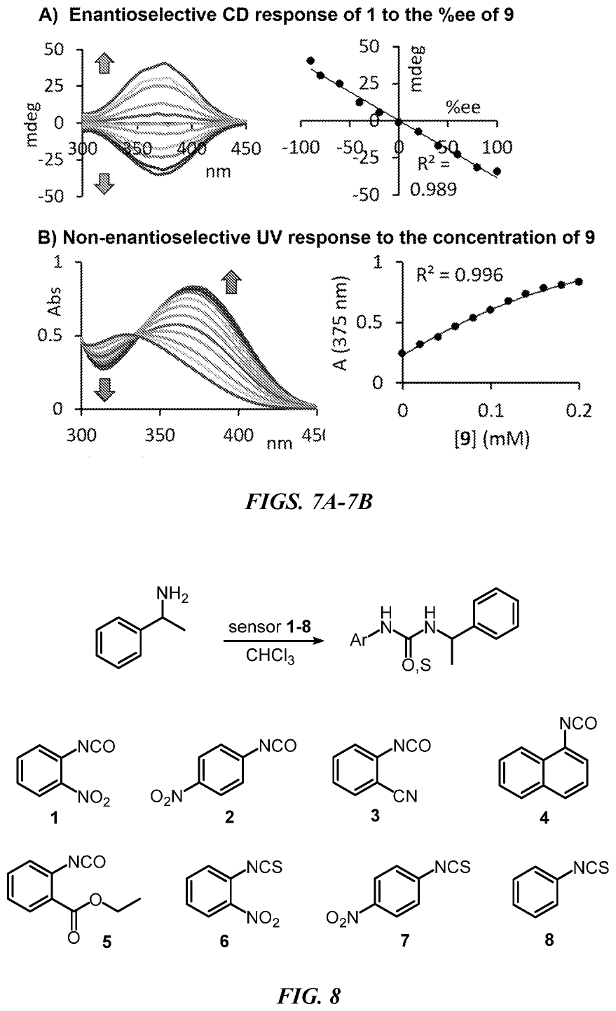

This was able to be verified by CD and UV studies of samples containing either 1-(2-naphthyl)ethanol, 31 (see FIGS. 6C-6D), or the initial test analyte 1-phenylethylamine, 9 (see FIGS. 7A-7B). In this example, CD spectra were collected with a standard sensitivity of 100 mdeg, a data pitch of 0.5 nm, and a bandwidth of 1 nm in a continuous scanning mode with a scanning speed of 500 nm/min and a response of 1 s (1 cm path length). The data were baseline corrected and smoothed using a binomial equation. UV spectra were collected with an average scanning time of 0.0125 s, a data interval of 1.00 nm, and a scan rate of 4800 nm/s.

The change in the UV absorbance upon addition of (R)-phenylethylamine, 9, to sensor 1 was measured. Sensor 1 (120.0 mM) and (R)-phenylethylamine, 9, in varying concentrations (0, 10, 20, 30, 40, 50, 60, 70, 80, 90, and 100 mM) were dissolved in 1.0 mL of CHCl₃ and stirred for 15 minutes. Each mixture was diluted with ACN (4.0 μL aliquot added to 2.0 mL of ACN) for UV analysis. The absorbance wavelength shifted from 328 nm to 375 nm as the substrate concentration increased from 0 to 100 mM. Plotting the intensity at 375 nm versus each concentration of phenylethylamine yielded a polynomial with R²=0.9961 and y=−3E−05x²+0.009x+0.2261 (FIG. 7B).

The change in the CD amplitude of sensor 1 upon addition of phenylethylamine in varying enantiomeric composition was measured. Sensor 1 (99.0 mM) and phenylethylamine (total 82.5 mM) with varying ee 's (+100, +80, +60, +40, +20, 0, −20, −40, −60, −80, and −100%) were dissolved in 1.0 mL of CHCl₃ and stirred for 15 minutes. Each mixture was diluted with ACN for CD analysis (10.0 μL aliquot added to 2.0 mL of ACN). Plotting the CD amplitude at 375 nm against the enantiomeric excess of phenylethylamine yielded a straight line with R²=0.9885 and y=−0.3868x+0.2678 (FIG. 7A).

Ten scalemic samples of phenylethylamine at varying concentrations and ee in CHCl₃ were prepared and subjected to simultaneous analysis of the concentration, enantiomeric excess, and absolute configuration using sensor 1. First, a UV spectrum was obtained as described above and the concentration was calculated using the intensities at 375 nm and the equation shown in FIG. 7B. Then, a CD spectrum was obtained as described above. The CD intensities were normalized to the concentrations obtained from UV analysis and the enantiomeric ratio was calculated using the intensities at 375 nm and the equation shown in FIG. 7A. The absolute configuration was determined using the sign of the Cotton effect. The results, presented in Table 1 below, show relatively small error margins that are generally considered acceptable for chiroptical sensing applications (Herrera et al., “Optical Analysis of Reaction Yield and Enantiomeric Excess: A New Paradigm Ready for Prime Time,” J. Am. Chem. Soc. 140:10385-10401 (2018), which is hereby incorporated by reference in its entirety). For example, the determination of an er of 1.1 (S):98.9 (R) and a total concentration of 62.1 mM for a 60.0 mM sample with 2.0% of the S-enantiomer and 98.0% of (R)-9 (see Table 1, entry 4). The UV/CD analysis of a solution of 9 at 25.0 mM with an SIR ratio of 10.0:90.0 gave 21.0 mM and an enantiomeric composition of 8.1:91.9 (see Table 1, entry 9).

TABLE 1
Results of Comprehensive Chiroptical Concentration and Enantiomeric
Ratio Sensing of 1-Phenylethylamine Samples Using Sensor 1.

| | Sample compositions | | | Optical sensing results | | |
|---|---|---|---|---|---|---|
| | Abs. | Conc. | | Abs. | Conc. | |
| Entry | Config. | (mM) | S/R | Config. | (mM) | S/R |
| 1 | R | 100.0 | 40.0:60.0 | R | 94.5 | 41.6:58.4 |
| 2 | S | 100.0 | 75.0:25.0 | S | 96.9 | 78.7:21.3 |
| 3 | S | 75.0 | 82.0:18.0 | S | 68.2 | 88.7:11.3 |
| 4 | R | 60.0 | 2.0:98.0 | R | 62.1 | 1.1:98.9 |
| 5 | S | 50.0 | 95.0:5.0 | S | 50.1 | 96.9:3.1 |
| 6 | R | 50.0 | 35.0:65.0 | R | 54.4 | 33.9:66.1 |
| 7 | S | 40.0 | 70.0:30.0 | S | 35.5 | 68.8:31.2 |
| 8 | R | 80.0 | 22.0:78.0 | R | 70.3 | 23.8:76.2 |
| 9 | R | 25.0 | 10.0:90.0 | R | 21.0 | 8.1:91.9 |
| 10 | S | 25.0 | 80.0:20.0 | S | 22.3 | 78.7:21.3 |

The concentrations and enantiomeric ratios were determined using the sensor UV and CD responses at 375 nm. The absolute configuration was assigned by comparison of the observed Cotton effects to a reference sample.

The change in CD amplitude of sensor 1 upon addition of 1-(2-naphthyl)ethanol, 31, in varying enantiomeric composition was measured. In a glovebox, sensor 1 (120.0 mM), DMAP (20.0 mM), and 1-(2-naphthyl)ethanol (total 100 mM) with varying ee 's (+100, +80, +60, +40, +20, 0, −20, −40, −60, −80, and −100%) were dissolved in 1.0 mL of CHCl₃ and stirred overnight. Each mixture was diluted with ACN for CD analysis (15.0 μL added to 2.0 mL of ACN). Plotting the CD amplitude at 350 nm against the enantiomeric excess of 1-(2-naphthyl)ethanol yielded a straight line with R²=0.997 and y=0.9783x+2.528 (FIG. 6C).

The change in the UV absorbance upon addition of (R)-1-(2-naphthyl)ethanol to sensor 1 was also measured. In a glovebox, sensor 1 (120.0 mM) and (R)-1-(2-naphthyl)ethanol in varying concentrations (0, 20, 40, 60, 80, and 100 mM) were dissolved in 1.0 mL of CHCl₃ and stirred overnight. Each mixture was diluted with ACN (5.0 μL aliquot added to 2.0 mL of ACN) for UV analysis. The absorbance wavelength shifted from 319 nm to 354 nm as the substrate concentration increased from 0 to 100 mM. Plotting the intensity at 350 nm versus each concentration of 1-(2-naphthyl)ethanol yielded a polynomial with R²=0.9989 and y=−2E−05x²+0.0067x+0.309 (FIG. 6D).

Finally, crystallographic analysis of (S)-1-(2-Nitrophenyl)-3-(1-phenylethyl)urea (FIG. 5A) and (R)-1-(Naphthalen-2-yl)ethyl (2-nitrophenyl)carbamate (FIG. 5B) was carried out. A single crystal was obtained by slow evaporation of a solution of the chiral product in dichloromethane:hexanes (1.5:1). Single crystal X-ray analysis was performed at 100 K using Bruker APEX DUO equipped with a Cu-Kα (λ=0.154178 Å) microfocus source, an ApexII detector, and an Oxford 700 Cryostream. Data were integrated with the Bruker SAINT program. Structure solution and refinement were performed using the SHELXTL/PC suite and ShelXle. Intensities were corrected for Lorentz and polarization effects and an empirical absorption correction was applied using Blessing's method as incorporated into the program SADABs. Non-hydrogen atoms were refined with anisotropic displacement parameters. (S)-1-(2-Nitrophenyl)-3-(1-phenylethyl)urea: C₁₅H₁₅N₃O₃, M=285.30, yellow needle, 0.034×0.082×0.529 mm³, monoclinic, space group P21, a=4.64130(10), b=11.0456(3), c=13.6158(4) Å, V=693.65(3) Å3, Z=2. Absolute structure parameter=0.2(2). (R)-1-(Naphthalen-2-yl)ethyl (2-nitrophenyl)carbamate: C₁₉H₁₆N₂O₄, M=336.34, yellow block, 0.258×0.307×0.467 mm³, orthorhombic, space group P212121, a=5.8533(12), b=7.9653(17), c=34.781(8) Å, V=1621.6(6) Å3, Z=4. Absolute structure parameter=−0.9(5).

As demonstrated herein, the urea and carbamate formations generate a strong CD signal with a maximum at 375 nm and 350 nm, respectively, as well as a large UV change at the same wavelength. This sets the stage for comprehensive UV/CD sensing of the concentration and enantiomeric ratio of chiral compounds. It is important to note that CD spectrophotomers typically produce UV and CD spectra simultaneously, which makes this approach even more attractive.

### Discussion of Examples 1-3

Examples 1-3 show for the first time that a simple aryliso(thio)cyanate probe enables optical concentration and er determination of chiral compounds based on fast mixing followed by straightforward UV/CD analysis. The general scope and ease of operation are unprecedented to date, and demonstrated with readily available 2-nitrophenylisocyanate as sensor and almost 40 analytes representing 5 different compound classes. The sensor reacts smoothly and irreversibly with amino and alcohol groups at room temperature toward urea or carbamate products exhibiting characteristic UV and CD signals above 300 nm that are correlated to the total concentration as well as the enantiomeric composition of the target compound. The use of such a broadly useful achiral chiroptical agent is very attractive and it combines several features and advantages that outperform current laboratory practice:
(1) It eliminates complications that can arise when traditional methods are used, such as kinetic resolution effects and interferences from enantiomeric contamination of chiral derivatizing agents, as well as cumbersome work-up procedures and time-consuming chromatographic protocols that impede fast and accurate enantiomer analysis.
(2) The in situ generation of distinct UV and CD signals at high wavelengths overcomes issues with insufficient resolution of overlapping signals often encountered with chiral NMR solvating agents that rely on weak binding forces rather than covalent bond formation.
(3) The compatibility with a variety of solvents was another noteworthy and important characteristic of this assay. It addresses problems with insufficient solubility of very polar analytes, for example pharmaceuticals, in standard mobile phase mixtures required for chiral HPLC analysis, which regularly causes major roadblocks in analytical laboratories.
(4) Finally, the elimination of any work-up and chromatographic separation steps saves time and solvent consumption. Therefore, it was believed that the broad application spectrum, ruggedness and practicality of chiroptical sensing with aryliso(thio)cyanate probes together with the availability of automated CD multi-well plate readers carry exceptional promise to accelerate chiral compound development projects at reduced cost and with less waste production.

### Example 4—Chirality Sensing of Amines with Quinones

Based on their versatility, quinones exhibiting replaceable halide or pseudohalide substituents were evaluated to assess whether they would allow smooth covalent capture of a variety of nucleophilic chiral compounds and, thus, trigger a strong chiroptical response originating from the proximate positioning of the molecular asymmetry and its chromophoric ring structure. This chemistry was expected to occur fast and quantitatively under mild conditions with preservation of the fully conjugated dione structure. The substitution of an electron-withdrawing substituent by an electron donor, for example an amine, should significantly alter the optical properties, partly as a result of effective push-pull conjugation across the quinone scaffold. Because the carbon-nitrogen bond formation was based on an addition-elimination sequence, it does not generate a new chirality center. As a result, complicated diastereomeric mixtures are not be produced, which assures that the interpretation and quantitative analysis of any optical changes measured remain simple. With these chemical and optical design features in mind, whether the placement of a chiral amine or another suitable N-nucleophile directly at the quinone ring would induce strong UV and CD signals with potential for quantitative chirality sensing was evaluated.

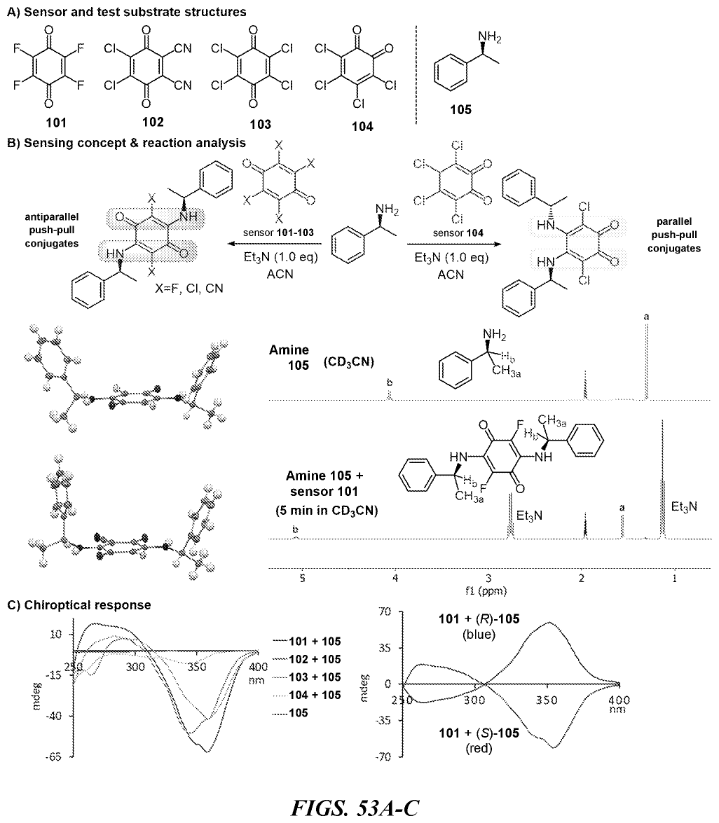

The suitability of quinone sensors was assessed using the readily available tetrasubstituted benzoquinones 101-104 and 1-phenylethylamine, 105, as the test substrate (FIG. 53A). Initial screening was carried out with (S)-1-Phenylethylamine (3.33 mM), Et₃N (3.33 mM) and a quinone sensor 101-104 (1.67 mM), which were mixed in 1.5 mL of ACN and the reaction was stirred overnight followed by CD analysis in 2.0 mL of ACN (0.13 mM). CD spectra were collected with a standard sensitivity of 100 mdeg, a data pitch of 0.5 nm, and a bandwidth of 0.5 nm in a continuous scanning mode with a scanning speed of 1000 nm/min and a response of 0.5 s (1 cm path length). The data were baseline corrected and smoothed using a binomial equation. The initial screening efforts showed that all quinones react with two molecules of 105 at room temperature, although sensor 101 yielded the strongest CD signal (FIG. 59). Thus, further optimization was carried out with sensor 101.

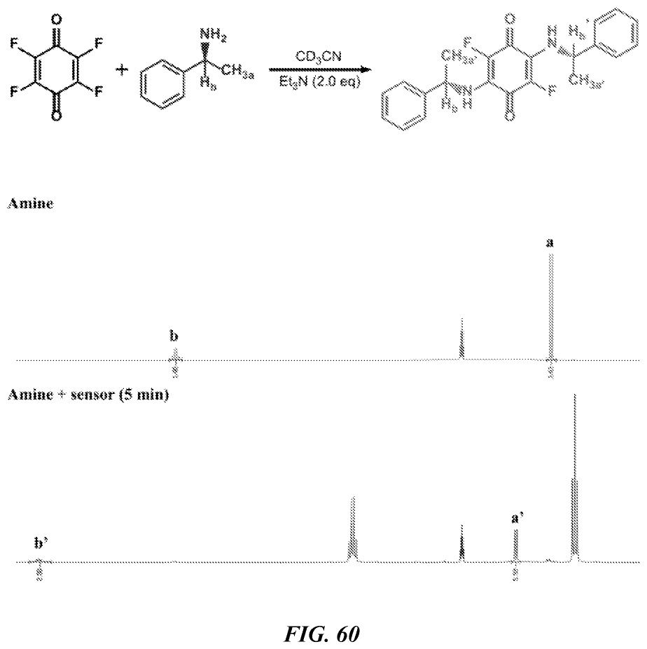

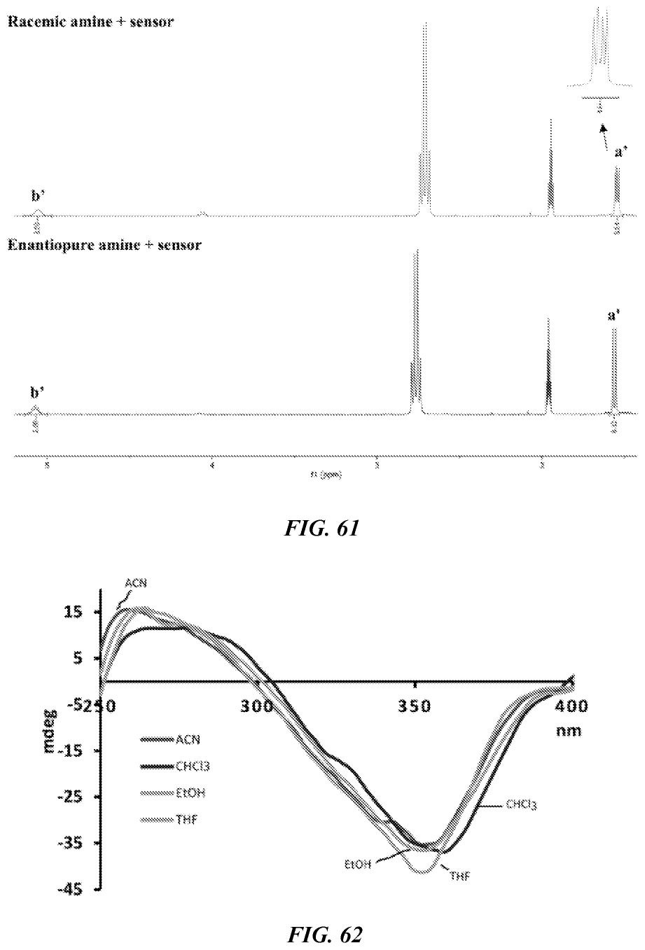

The reaction between (S)-1-phenylethylamine and quinone sensor 101 was monitored by ¹H NMR, which was carried out at 400 MHz using deuterated ACN as solvent. Chemical shifts were reported in ppm relative to the solvent peaks. (S)-Phenylethylamine (33.3 mM), Et₃N (33.3 mM) and sensor 101 (16.7 mM) were mixed in 1.5 mL of CD₃CN, and ¹H NMR confirmed that the reaction was completed within 5 minutes (see FIGS. 60-61), while detectable amounts of by-products were not observed (FIG. 53B).

Solvent optimization was carried out using (S)-1-phenylethylamine (33.3 mM), Et₃N (33.3 mM) and sensor 101 (16.7 mM), which were mixed for 5 minutes in either 1.5 mL of THF, 1.5 mL of ACN or 1.5 mL of CHCl₃, and then diluted to a final volume of 2.0 mL with ACN, CHCl₃, THF or EtOH to obtain a final concentration of 0.15 mM for CD analysis. Reactions run in ACN or THE gave stronger CD signals than reactions run in CHCl₃ (see FIGS. 62-64). Differences in CD signals were negligible between dilution solvents. Strong CD spectra were obtained when THE was used as the reaction and the dilution solvent. All subsequent reactions were conducted using THE as solvent unless noted otherwise.

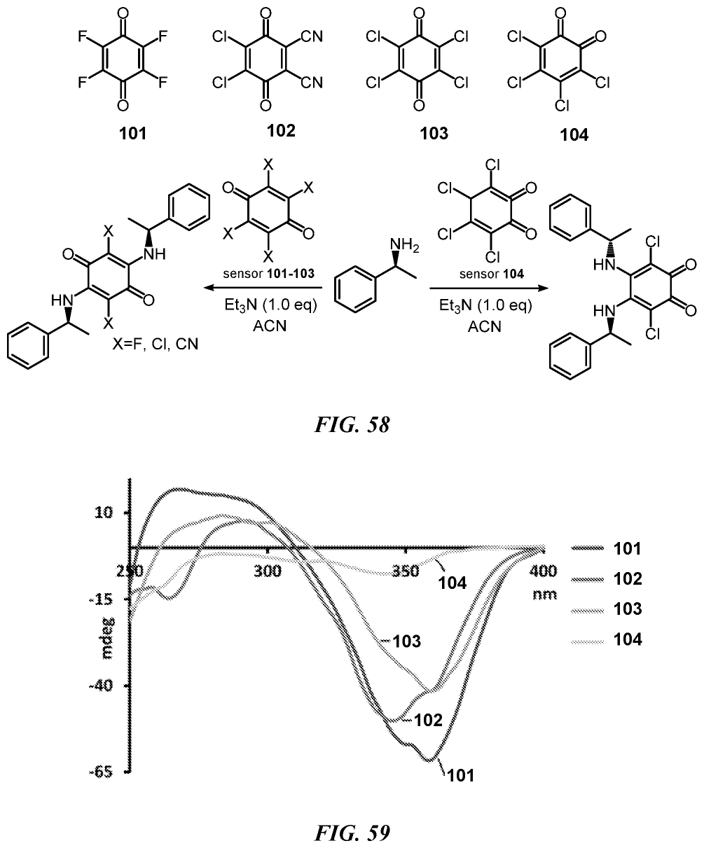

The click chemistry like nature of the reaction between 105 and 101-104 greatly facilitates chiroptical sensing applications as any work-up and product isolation was unnecessary (FIG. 58). The unaltered reaction mixtures obtained with (S)-105 and the quinone sensors in acetonitrile after a short stirring period were subjected directly to CD analysis after dilution to submillimolar concentrations with the same solvent (FIG. 53C). Strong negative Cotton effects at long wavelengths were observed with the paraquinones derived from 101-103 while the amination of the orthoquinone 104 gave relatively weak signals under identical conditions (FIG. 59). The R-enantiomer of 105 was then included in the same protocol and obtained the corresponding positive Cotton effect as expected (see FIGS. 53A-C; FIG. 59). Tetrafluorobenzo-quinone 101 gave the strongest chiroptical response upon binding of amine 105 (FIG. 59), and was chosen to further explore the general scope and utility of the sensing concept illustrated above. The reaction and the subsequent CD analysis can be completed within a few minutes and without any precautions.

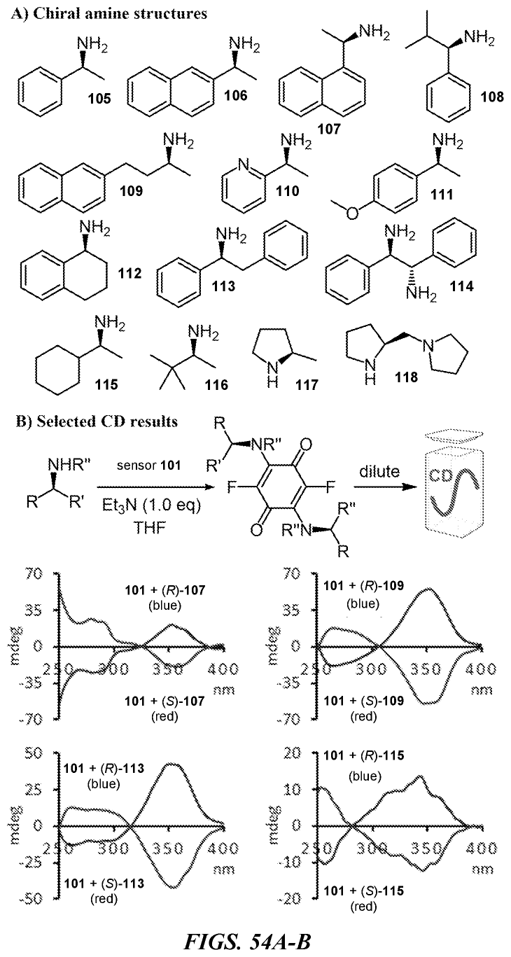

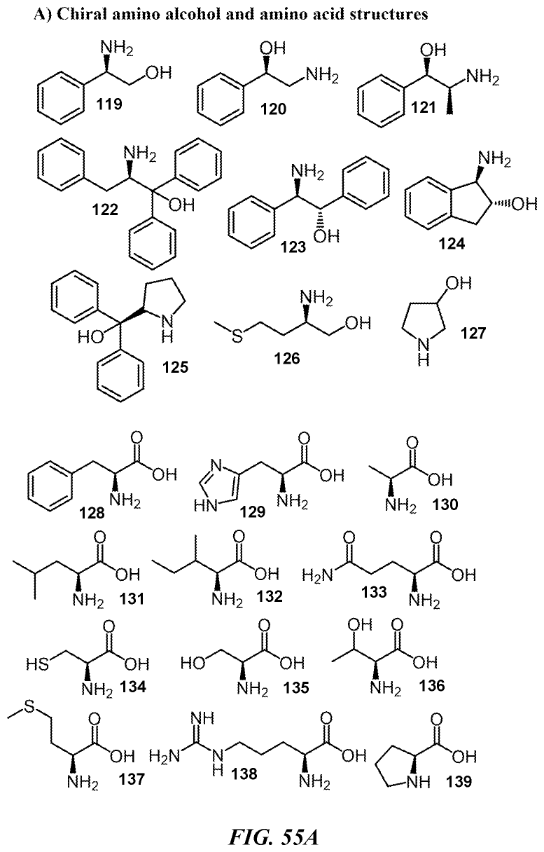

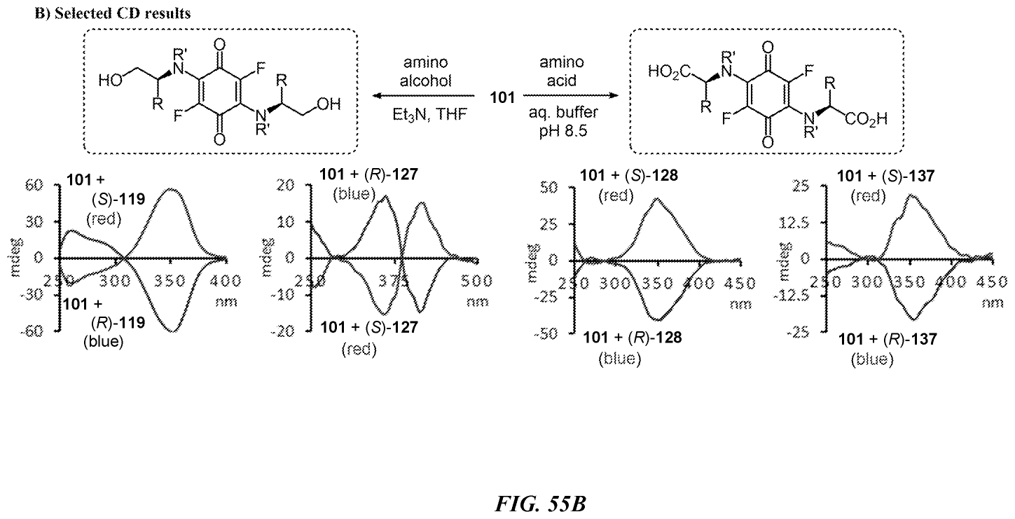

The CD assay was actually quite practical and robust; it can be conducted under air and in the presence of moisture or even in aqueous solutions if necessary. This enables sensing of free amines that do not need to be derivatized to improve solubility in organic solvents. Encouraged by this operational simplicity and the initial chirality sensing results with 105, continued testing of a variety of other amines occurred, some carrying an aromatic group which may contribute to the CD induction, for example via π-π stacking with the quinone moiety, but also the purely aliphatic substrates 115-118 (see FIGS. 54A; 65). For each of the amines, a solution of chiral amine 105-128 (3.33 mM), Et₃N (3.33 mM) and sensor 102 (1.67 mM) in 1.5 mL of THE was stirred for 5 minutes. CD analysis was performed after dilution with THE to the final concentration indicated in the figure descriptions (125-175 μL of the reaction mixture were diluted to 2.0 mL TIF) (FIGS. 54A-B; 66-79). Strong CD signals at long wavelengths were obtained with the same protocol in all 14 amines tested (FIGS. 54A-B; 66-79). It was noteworthy that the generation of a large CD amplitude above 300 nm was generally considered beneficial, because it eliminates possible interferences that may arise from the presence of chiral impurities during quantitative er analysis.

Finally, crystallographic analysis of (S,S)-2,5-difluoro-3,6-bis((1-phenylethyl)amino)-1,4-benzoquinone and (S,S)-2-chloro-5-cyano-3,6-bis((1-phenylethyl)amino)-1,4-benzoquinone was carried out. A single crystal of each was obtained by slow evaporation of a solution of either (S,S)-2,5-difluoro-3,6-bis((1-phenylethyl)amino)-1,4-benzoquinone or (S,S)-2-chloro-5-cyano-3,6-bis((1-phenylethyl)amino)-1,4-benzoquinone in dichloromethane:hexane (1:2 v/v). Single crystal X-ray analysis was performed at 100K using a Siemens platform diffractometer with graphite monochromated Mo-Kα radiation (k=0.71073 Å). Data were integrated and corrected using the APEX 3 program. The structures were solved by direct methods and refined with full-matrix least-square analysis using SHELXL-2017/1 or SHELXL-2018/3 software. Non-hydrogen atoms were refined with anisotropic displacement parameter. Crystal data: C₂₂N₂O₂F₂, M=382.4, 0.472×0.238×0.056 mm³, orthorhombic, space group P21, a=7.6011(9), b=8.3917(10), c=29.100(3) Å, V=1856.2(4) Å3, Z=4. Crystal data: C₂₃ClN₃O₂, M=405.87, 0.474×0.196×0.116 mm³, monoclinic, space group P21, a=11.1085(4), b=7.6821(2), c=11.5712(4) Å, V=968.12(5) Å3, Z=2.

### Example 5—Chirality Sensing of Amino Alcohols and Amino Acids with Quinones

The same protocol used in Example 4 was essentially extended to the amino alcohols 119-127, and was used with an aqueous acetonitrile pH 8.5 borate buffer solution for the sensing of amino acids 128-139 (FIGS. 55A-B, 80, 90). In accordance with Example 4, these 21 additional substrates were selected to encompass structures exhibiting a small aromatic ring as well as some that are devoid of a chromophore and therefore increasingly challenging. Nevertheless, distinct CD effects were obtained without exception just by fast mixing of the assay components and CD analysis without further delay.

For the amino alcohols 119-127, a solution of the chiral amino alcohol (3.33 mM), Et₃N (3.33 mM) and sensor 101 (1.67 mM) in 1.5 mL of tetrahydrofuran was stirred for 5 minutes. CD analysis was performed after dilution with THE to the final concentration indicated in the figure descriptions (125-200 μL of the reaction mixture were added to 2.0 mL THF) (FIGS. 55A-B; 80-89).

For the amino acids 128-139, a solution of sensor 101 (1.00 mM) and the amino acid (2.00 mM) in 2.5 mL of acetonitrile:aqueous pH 8.5 borate buffer (4:1 v/v, 10.0 mM) was stirred for 5 minutes. CD analysis was performed after dilution with ACN to the final concentration indicated in the figure descriptions (200-400 μL of the reaction mixture were diluted with 2.0 mL of ACN) (FIGS. 55A-B; 90-102).

### Example 6—Chirality Sensing for Combined er and Concentration Analysis

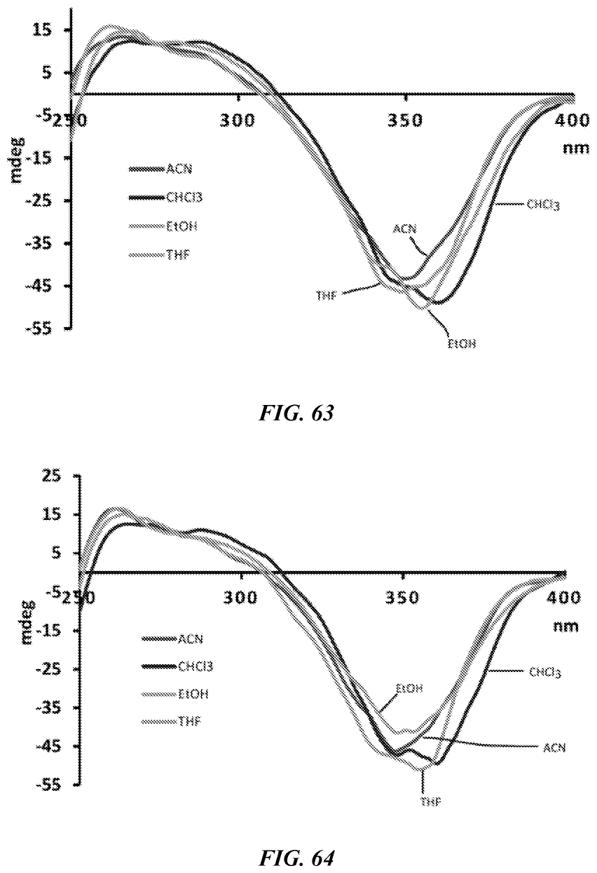

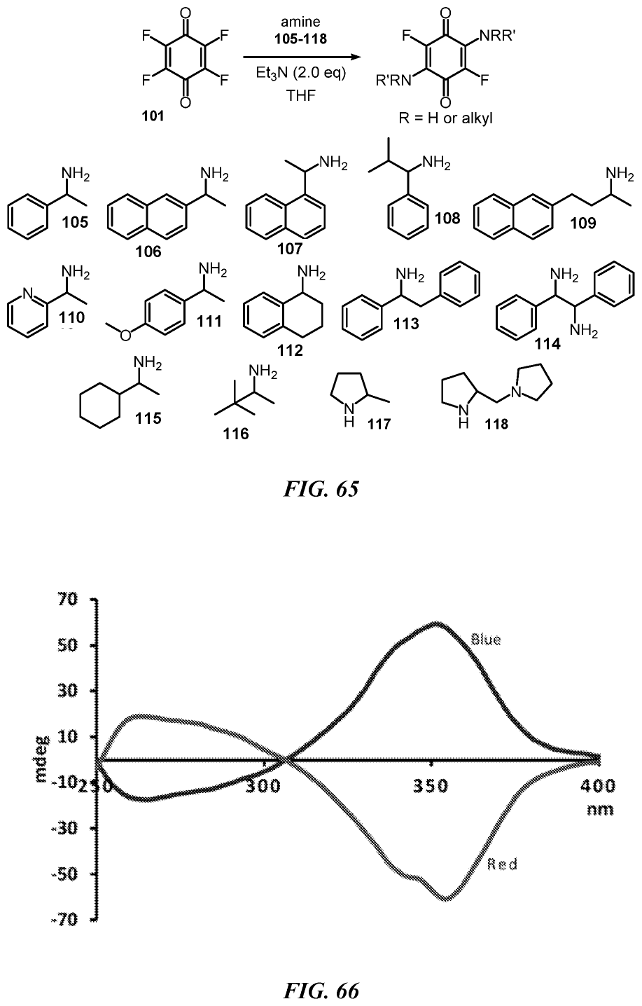

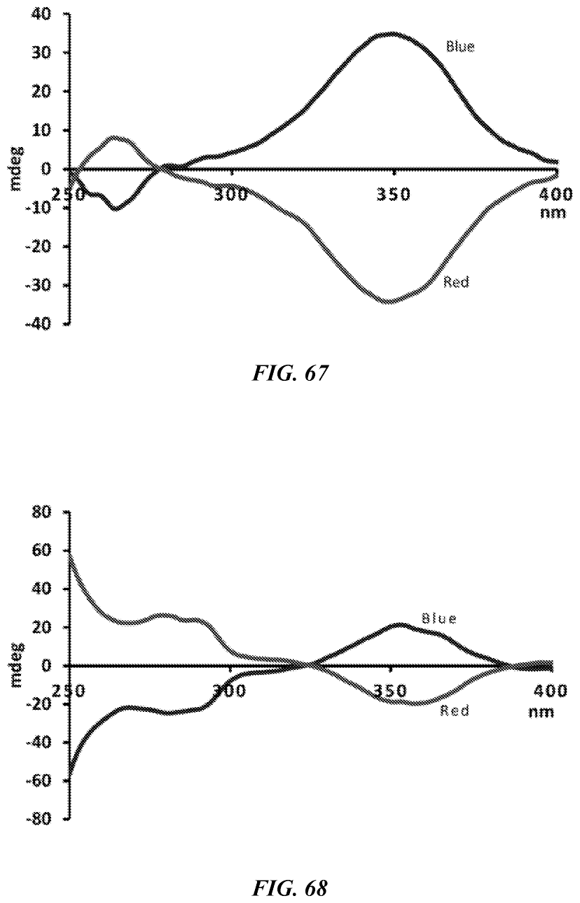

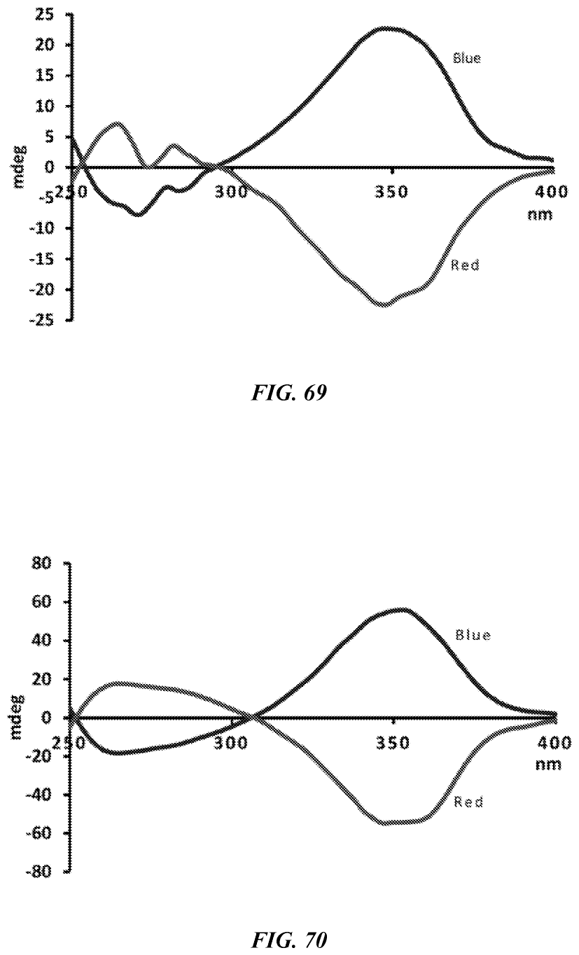

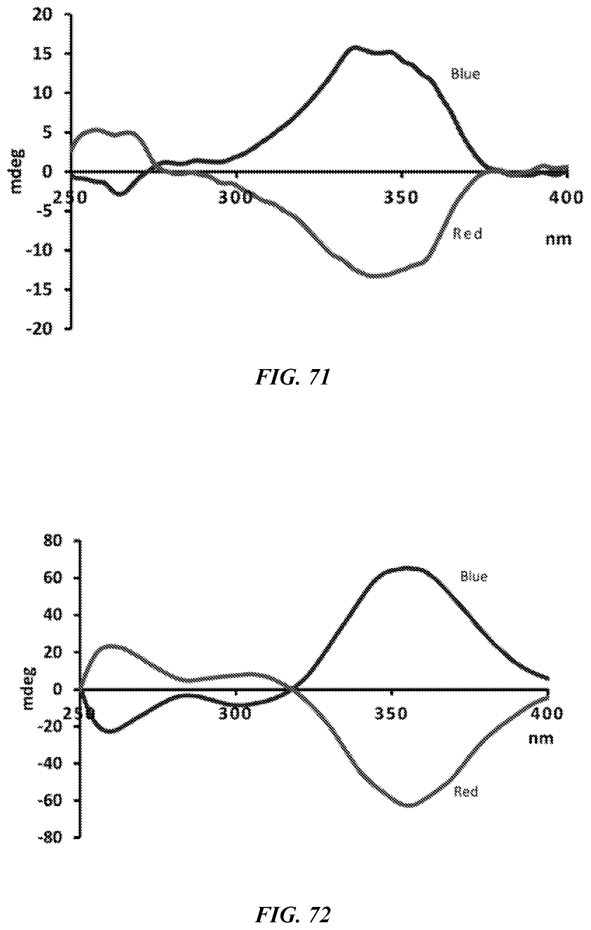

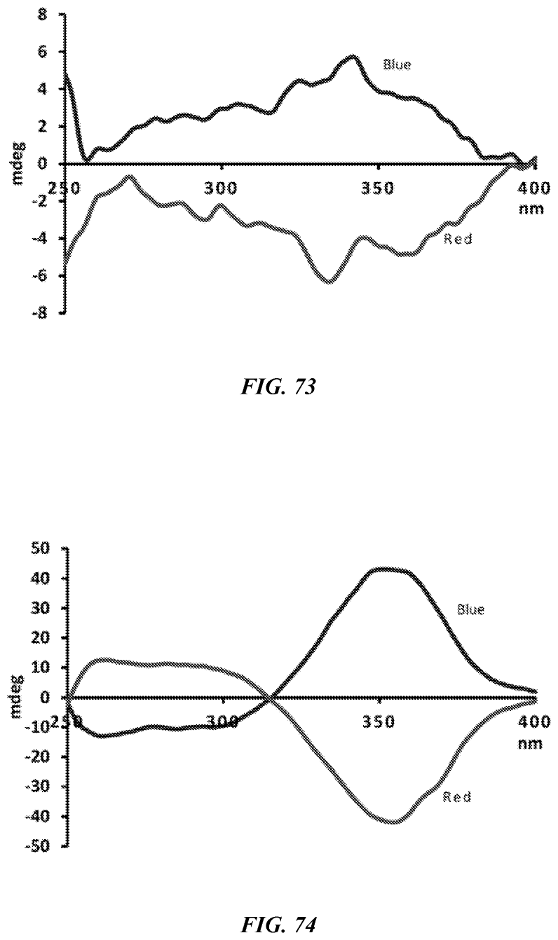

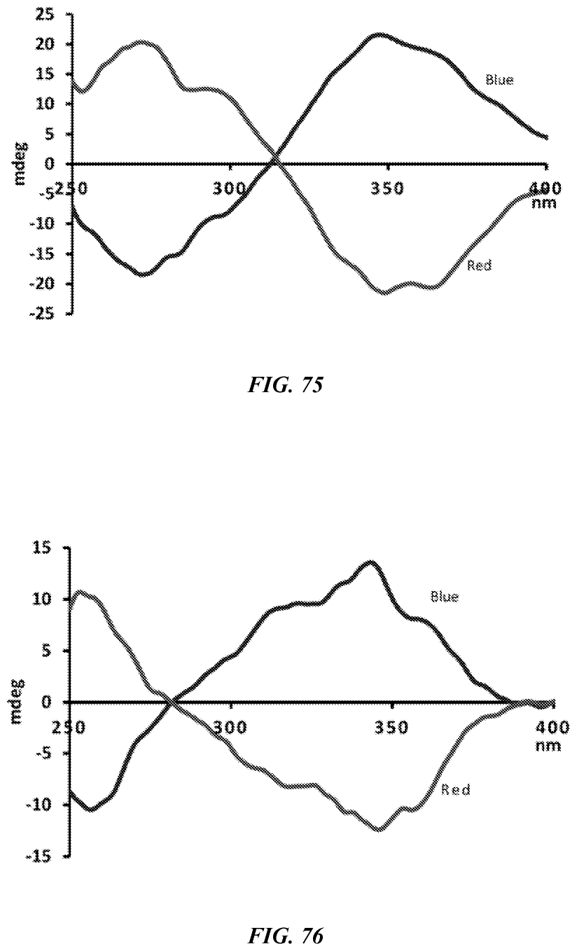

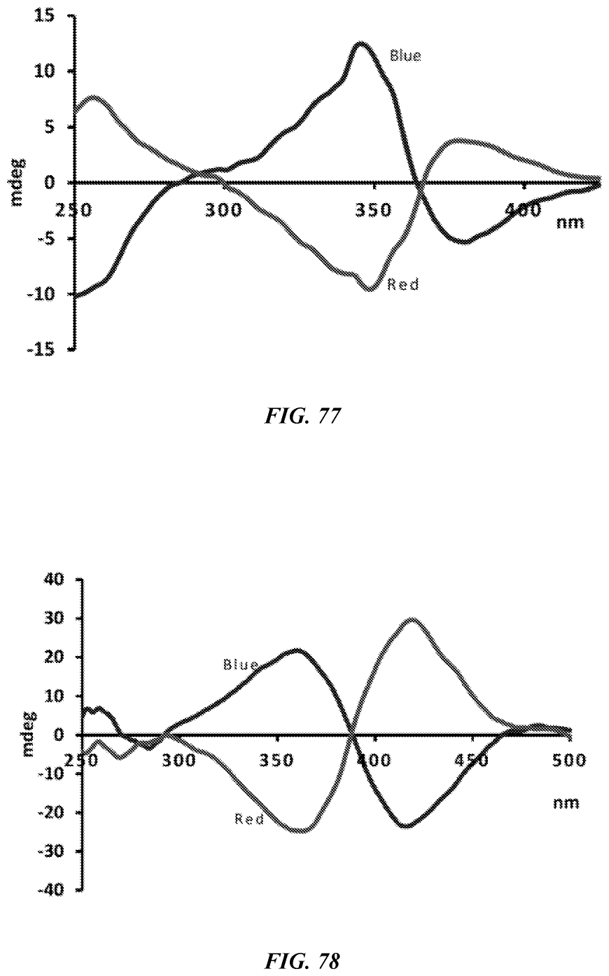

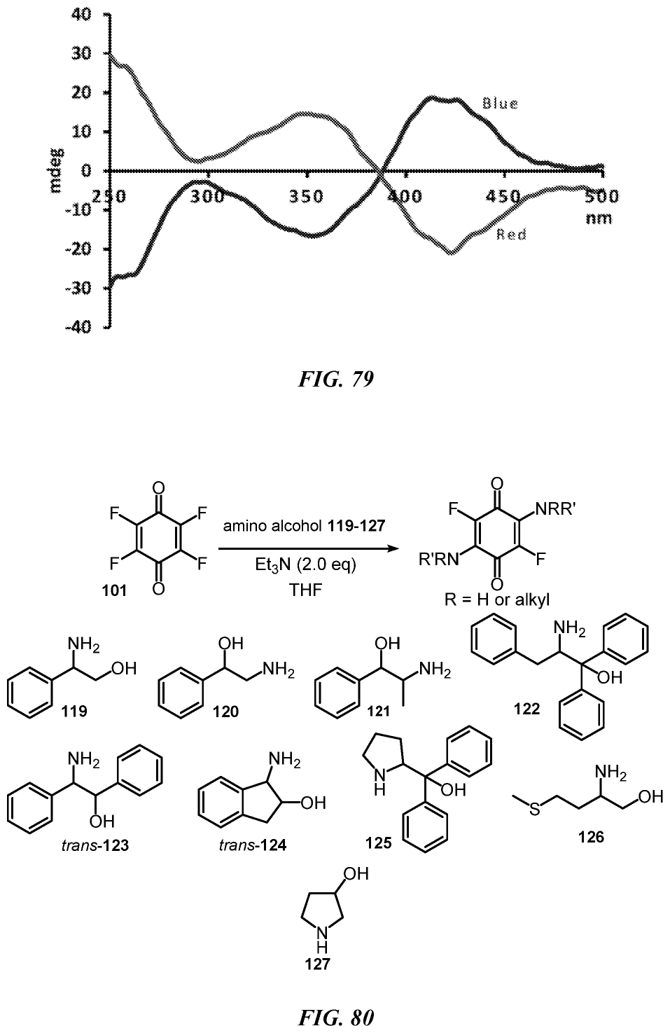

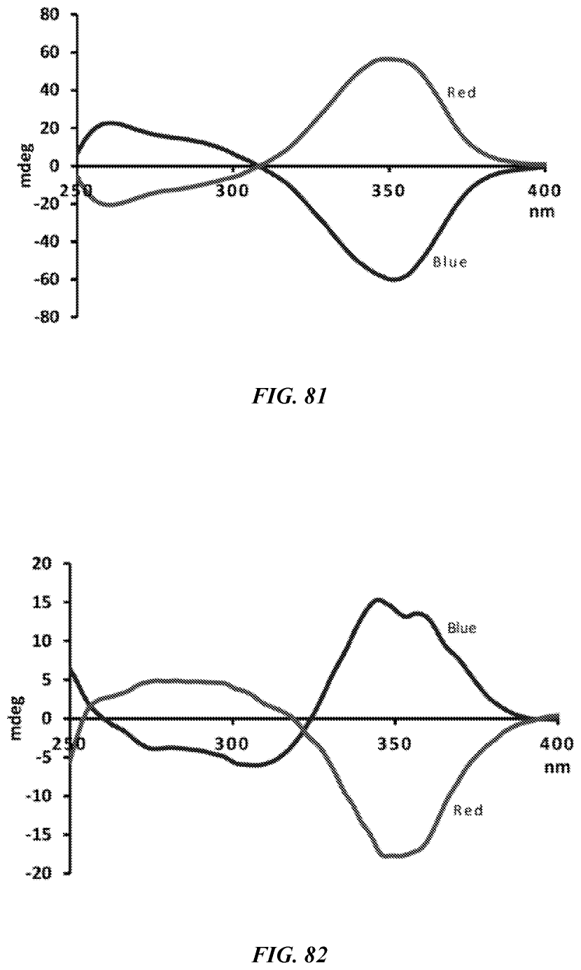

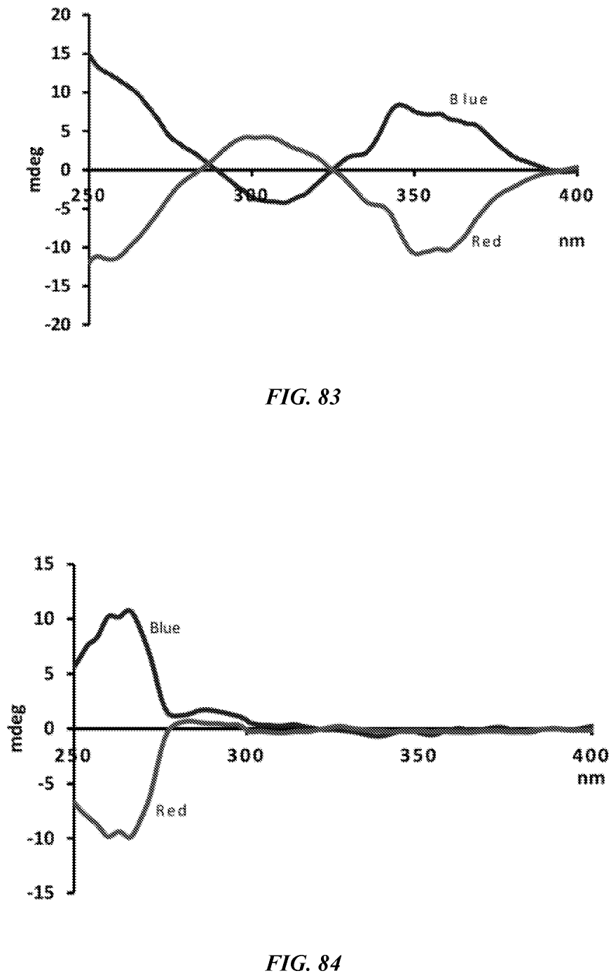

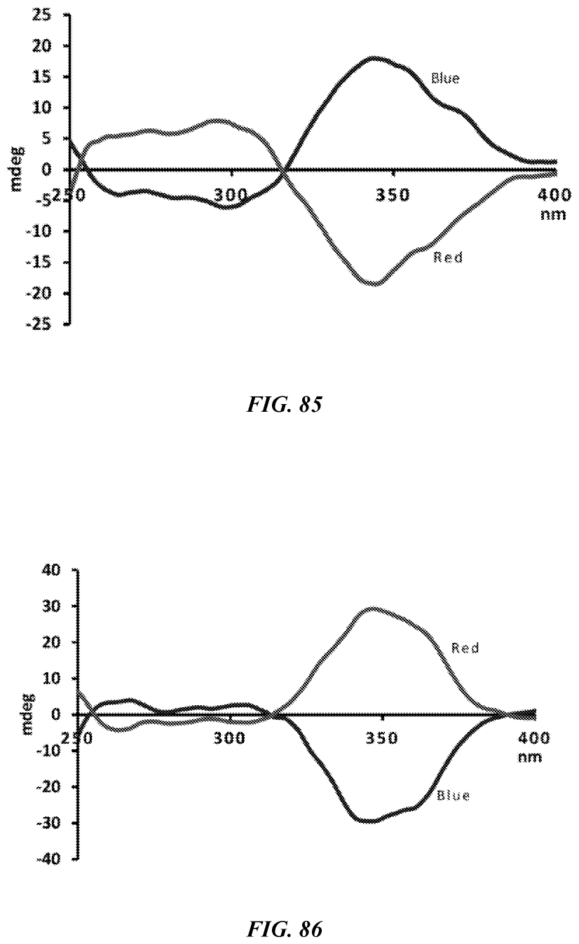

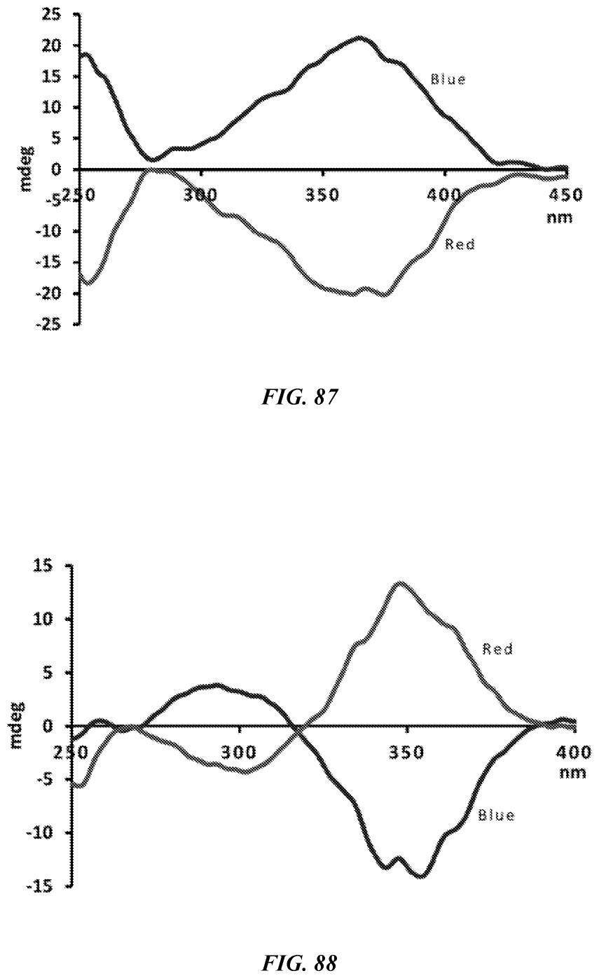

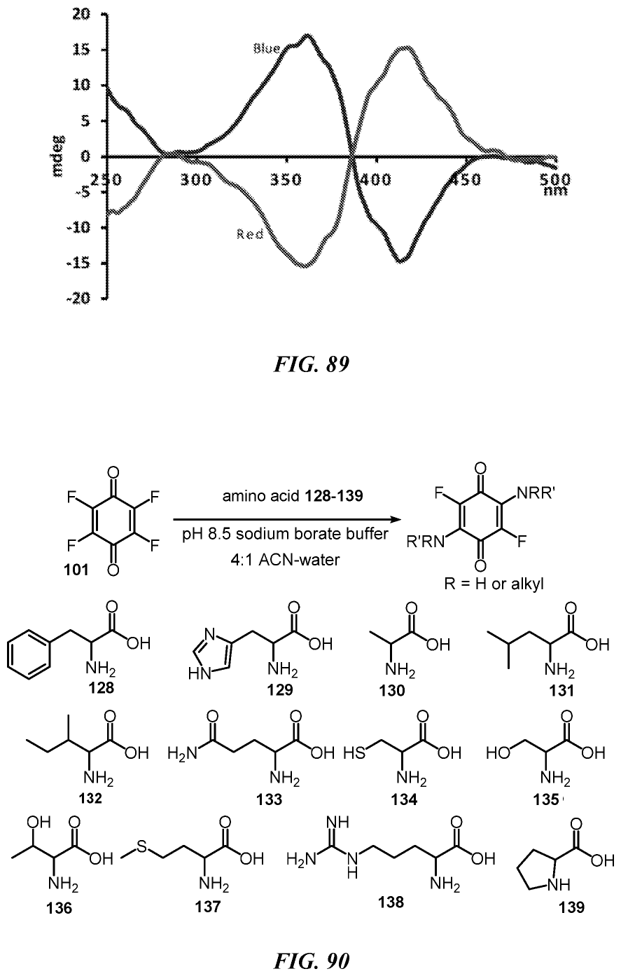

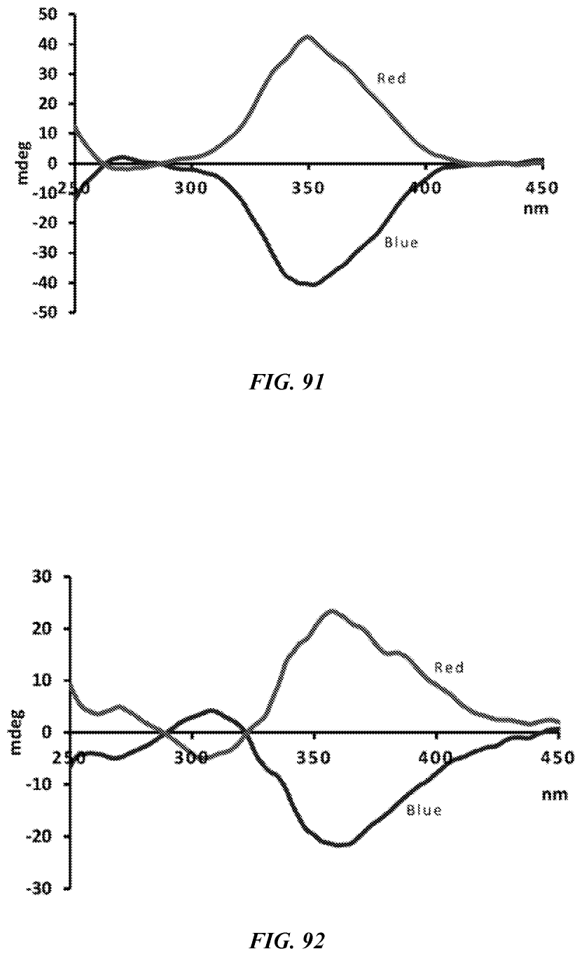

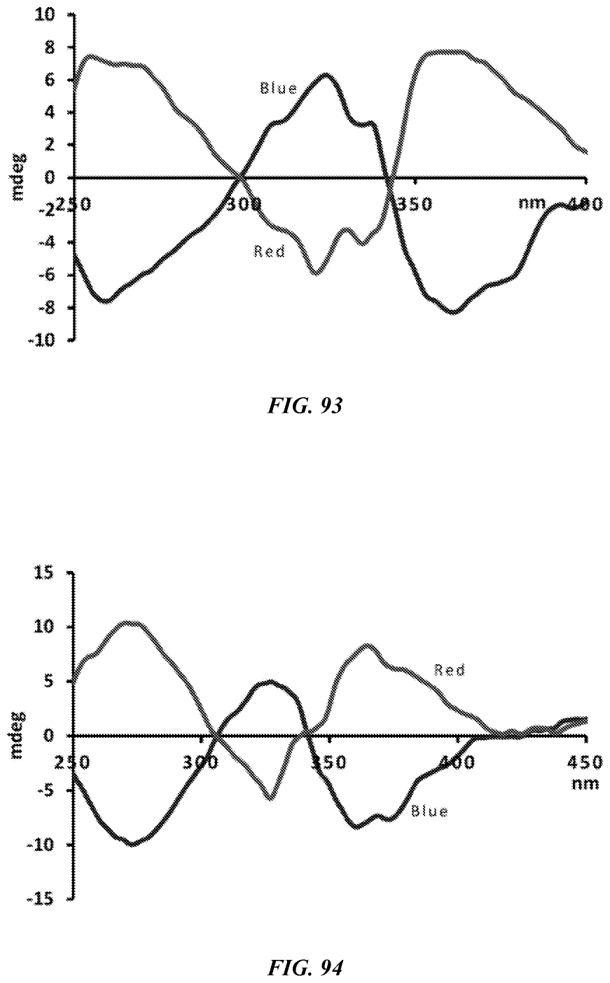

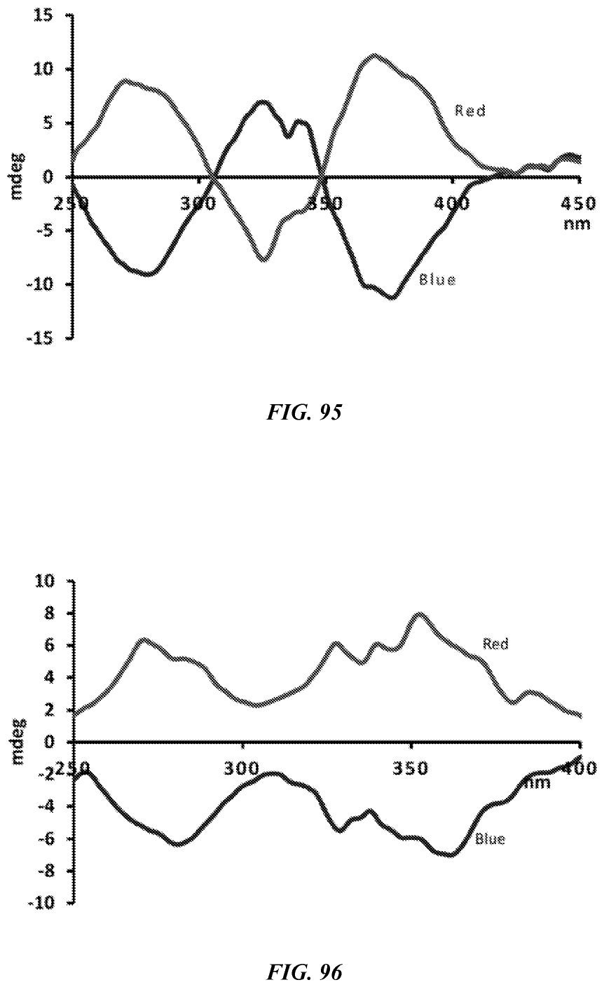

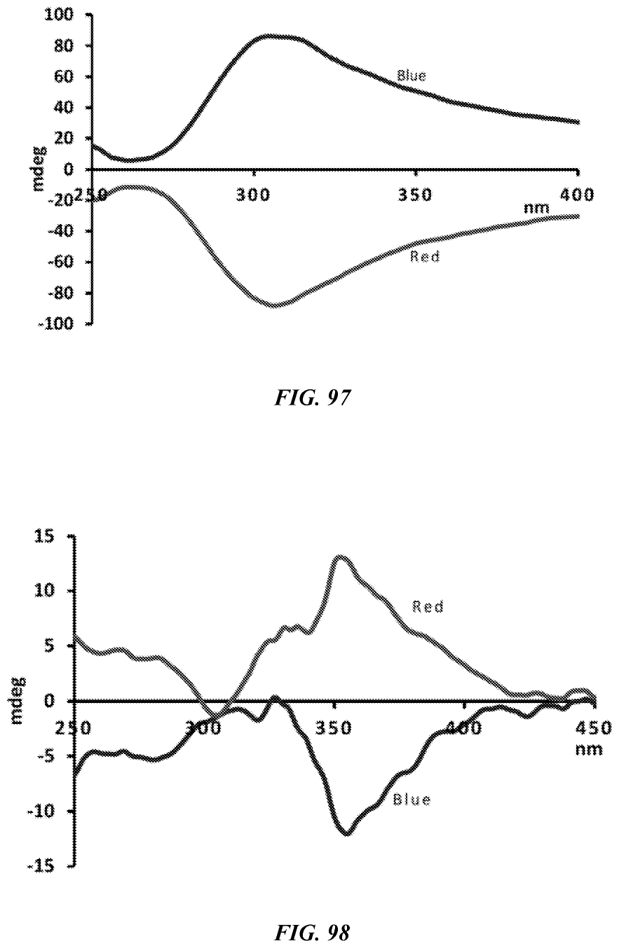

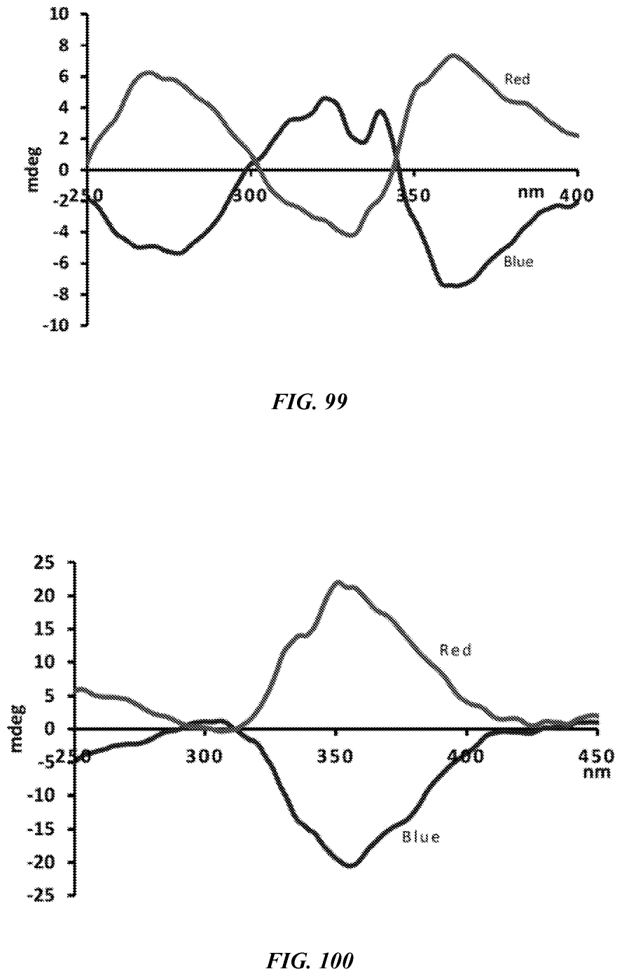

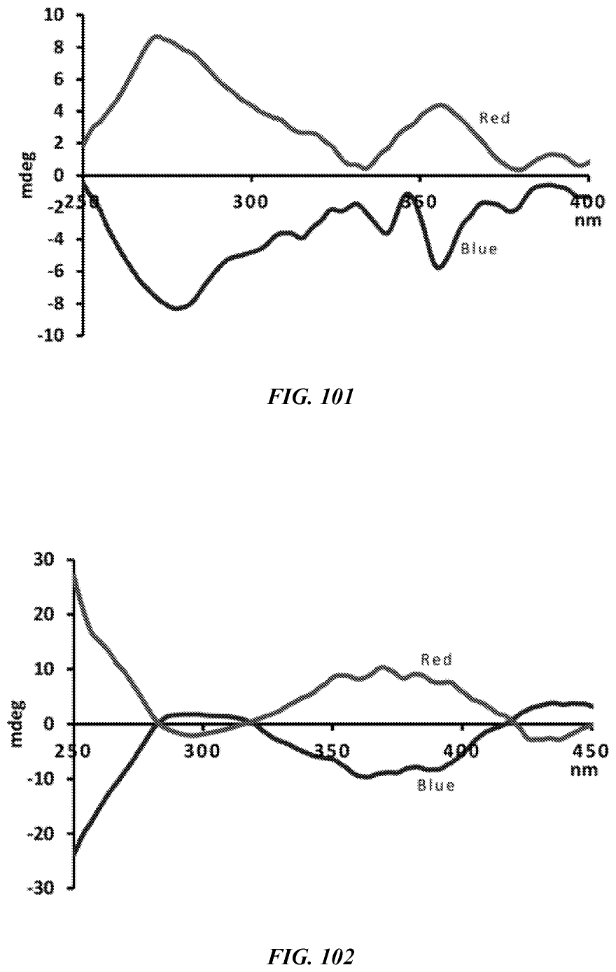

Having successfully applied the quinone 101 to a total of 35 structurally diverse compounds, the possibility of quantitative chirality sensing was investigated. This would be most impactful if simultaneous determination of the enantiomeric ratio and of the overall concentration of a chiral analyte were possible. Fortunately, it was found that the sensing reaction yields a strong UV change at ˜350 nm (FIGS. 55A-B). Because the formation of a new chirality center was avoided during the C—N bond formation the quantitative analysis of this steadily increasing UV signal was greatly simplified. In fact, this optical sensor response was not enantioselective, i.e. the same UV response was obtained irrespective of the enantiomeric sample composition. As a result, the profound CD signal induction can be correlated to the er and the characteristic UV change to the total concentration of both enantiomers, by using the same sample. This was practical and a user-friendly solution to a generally cumbersome task, because CD spectrophotometers typically generate CD and UV spectra together. Interestingly, the induced CD signal increases linearly with the enantiomeric excess of the sample. Because two analyte molecules are attached to the quinone core, one can expect a mixture of (R,R)-, (S,S)- and (R,S)-isomeric products when both enantiomers are present in the original sample. However, the latter was centrosymmetric and a meso compound that was naturally CD-inactive. NMR analysis of the reaction between 101 and racemic 105 showed that the three possible stereoisomeric 2,5-difluoro-3,6-bis((1-phenylethyl)amino)-1,4-benzoquinones are approximately formed in the statistically favored 1:2:1 ratio, indicating negligible asymmetric induction once the first amine was bound (FIG. 63).

With a thorough understanding of the mechanistic underpinnings of the chirality sensing assay, ten amine samples were prepared containing 105 in widely varying concentrations and enantiomeric ratios. These mixtures were applied in the general protocol described above for simultaneous UV/CD analysis.

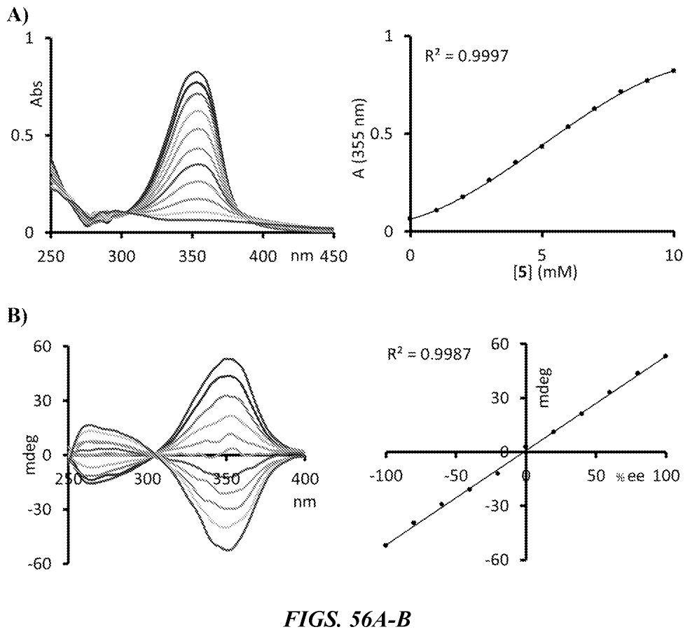

The change in the UV absorbance upon addition of sensor 1 to (S)-phenylethylamine and Et₃N was measured. Sensor 101 (5.0 mM), Et₃N (10 mM) and (S)-phenylethylamine in varying concentrations (0, 1, 2, 3, 4, 5, 6, 7, 8, 9, and 10 mM) were dissolved in 1.5 mL of THF and stirred for 5 minutes. Each mixture was diluted with THE (50.0 μL aliquot added to 2.0 mL of THF) for UV analysis. Plotting the intensity at 355 nm versus each concentration of phenylethylamine yielded a polynomial with R²=0.9997 and y=−0.0008x3+0.0119x2+0.0358x+0.0634 (FIG. 56A).

The change in the CD amplitude upon addition of sensor 101 to Et₃N and phenylethylamine in varying enantiomeric compositions was measured. Sensor 101 (1.67 mM), Et₃N (3.33 mM) and phenylethylamine (total 3.33 mM) with varying ee 's (+100, +80, +60, +40, +20, 0, −20, −40, −60, −80, and −100%) were dissolved in 1.5 mL of THF and stirred for 5 minutes. Each mixture was diluted with THE for CD analysis (125.0 μL aliquot diluted with 2.0 mL of THF). Plotting the CD amplitude at 350 nm against the enantiomeric excess of phenylethylamine yielded a straightline with R²=0.9987 and y=0.525x+0.8821 (FIG. 56B).

Ten scalemic samples of phenylethylamine at varying concentrations and ee in Et₃N were prepared and subjected to simultaneous analysis of the concentration, enantiomeric excess, and absolute configuration using sensor 101. First, a UV spectrum was obtained as described above and the concentration was calculated using the intensities at 355 nm and the equation shown in FIG. 56A. Then, a CD spectrum was obtained as described above. The CD intensities were normalized to the concentrations obtained from UV analysis and the ee was calculated using the intensities at 355 nm and the equation shown in FIG. 56B. The absolute configuration was determined using the sign of the Cotton effect.

The sample concentrations and enantiomeric ratios were determined using the chiroptical signals at 350 nm, and the sign of the CD response was used to assign the absolute configuration of the major enantiomer by comparison with the previously obtained reference CD spectrum. The results, presented in Table 2 below, show that this worked well without exception. For example, it was determined an er of 20.7 (S):79.3 (R) and a total concentration of 4.8 mM for a 5.0 mM sample with 20.0% of the S-enantiomer and 80.0% of (R)-105 (see Table 2, entry 1). The UV/CD analysis of a solution of 105 at 6.0 mM with an SIR ratio of 82.0:18.0 gave 5.9 mM and an enantiomeric composition of 85.5:14.5 (see Table 2, entry 2).

TABLE 2
Determination of the concentration, enantiomeric ratio, and
absolute configuration of samples of phenylethylamine determined
by simultaneous UV and CD responses of sensor 101.

| | Sample composition | | | Sensing results | | |
|---|---|---|---|---|---|---|
| | Abs. | Conc. | | Abs. | Conc. | |
| Entry | Config. | (mM) | S/R | Config. | (mM) | S/R |
| 1 | S | 5.0 | 20.0:80.0 | S | 4.8 | 20.7:79.3 |
| 2 | R | 6.0 | 82.0:18.0 | R | 5.9 | 85.5:14.5 |
| 3 | S | 6.5 | 60.0:40.0 | S | 7.0 | 60.6:39.4 |
| 4 | R | 7.0 | 70.0:30.0 | R | 7.0 | 72.5:27.5 |
| 5 | S | 7.5 | 90.0:10.0 | S | 7.8 | 97.6:2.4 |
| 6 | R | 8.0 | 35.0:65.0 | R | 8.2 | 32.6:67.4 |
| 7 | S | 8.0 | 25.0:75.0 | S | 8.0 | 18.8:81.2 |
| 8 | S | 4.0 | 22.0:78.0 | S | 3.6 | 21.3:78.7 |
| 9 | S | 8.0 | 5.0:95.0 | S | 8.1 | 2.0:98.0 |
| 10 | R | 5.0 | 60.0:40.0 | R | 4.6 | 58.0:42.0 |

The concentrations and enantiomeric ratios were determined using the sensor UV and CD responses at 350 nm. The absolute configuration was assigned by comparison of the observed Cotton effects to a reference sample.

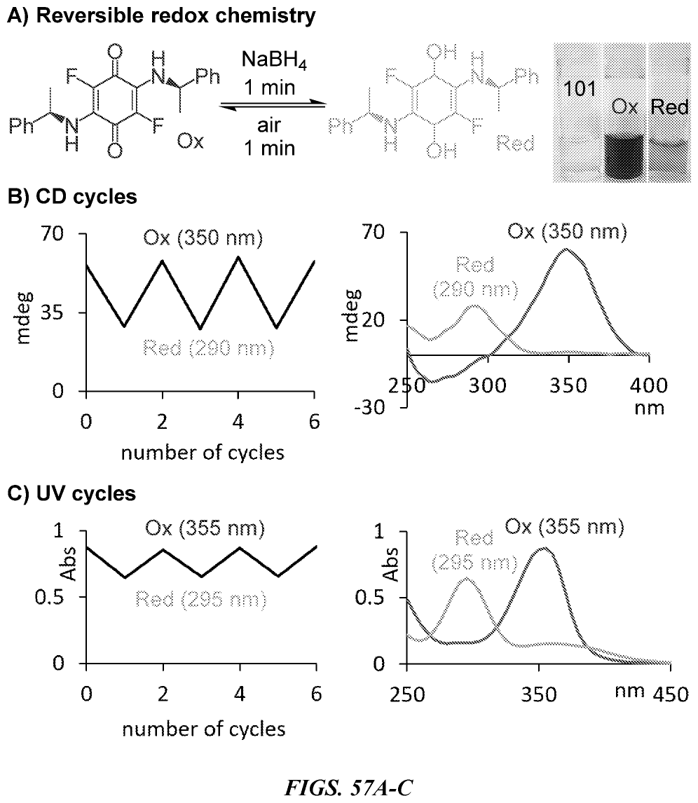

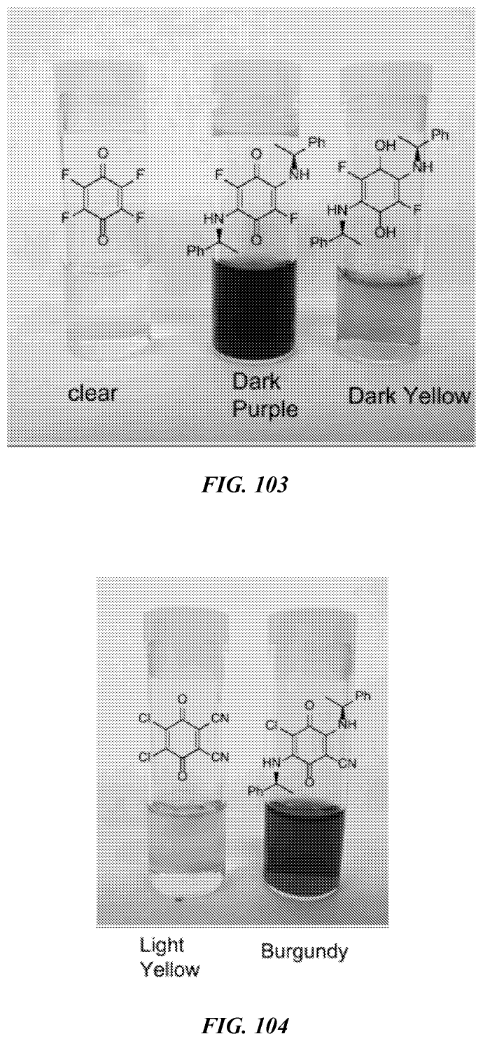

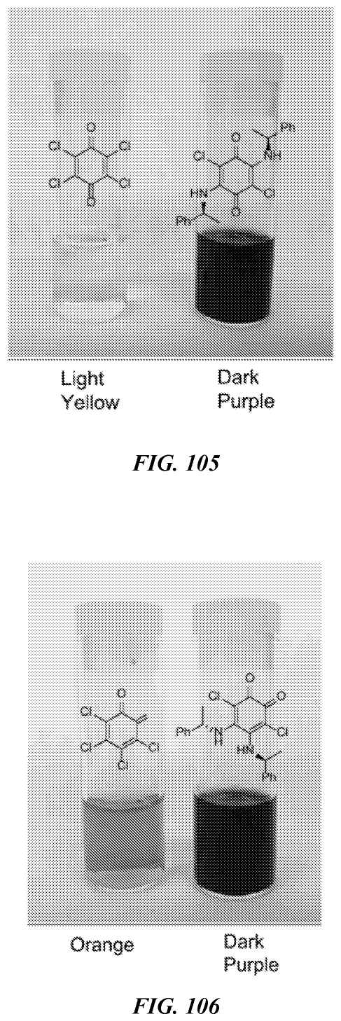

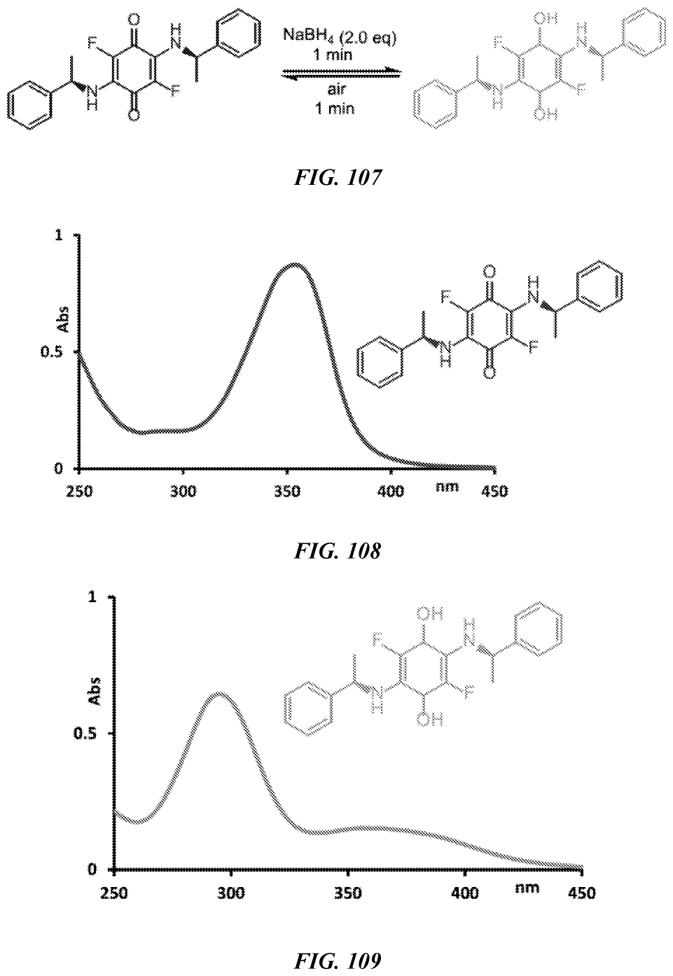

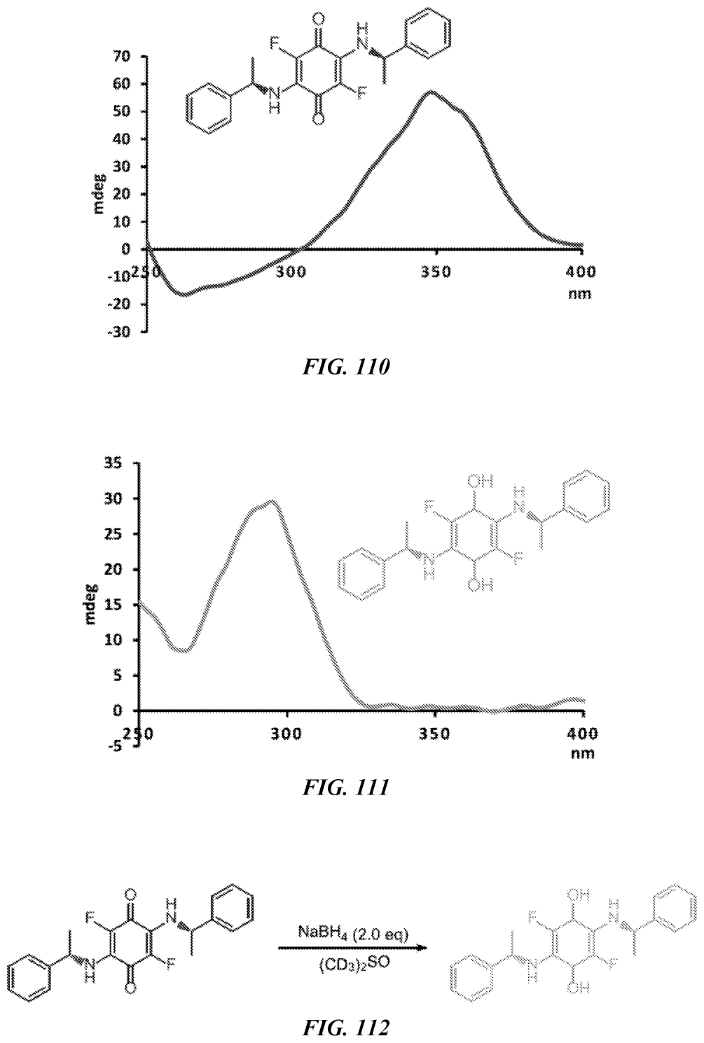

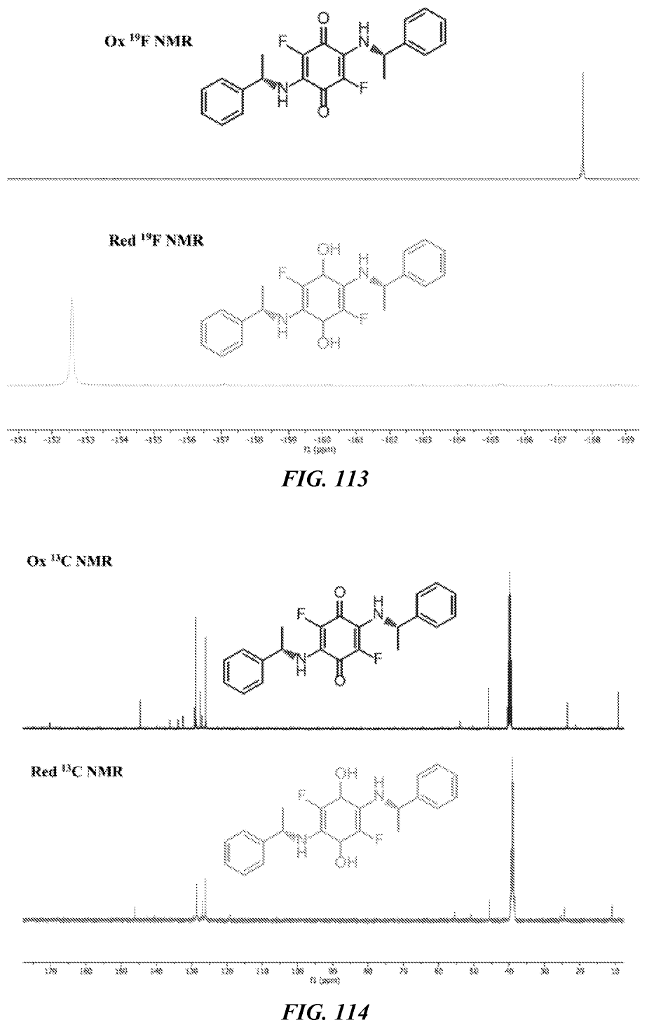

The reaction between the quinones 101-104 and 1-phenylethylamine, 105, coincides with a drastic color change. For example, the off-white color of a solution of 101 in THE turns to dark purple within five minutes upon addition of 105 and trimethylamine (see FIGS. 57A-C). After screening several reducing agents and conditions it was found that reduction of the push-pull quinone conjugate with sodium borohydride immediately gives a dark yellow solution which was assigned to the corresponding hydroquinone by ¹H, ¹³C and ¹⁹F NMR analysis (FIGS. 103-108). Exposure of the solution to air regenerated the original color within a minute (FIG. 107). The reversibility, short response time and the impressive colorimetric changes encouraged the applicant to further investigate chiroptical switching with this system. A reaction mixture of sensor 101, (R)-phenylethylamine and Et₃N was subjected to reduction/oxidation cycles using NaBH₄ and air, respectively, in a THF/methanol solution. The optical changes were measured by UV and CD spectroscopy (FIGS. 57A-C; 108-111). The oxidated state, which was visible by the dark purple color, shows a strong UV absorption at 355 nm. The reduction produces a new major UV absorption at 295 nm and a minor one at 375 nm. Re-oxidation was achieved in less than one minute by agitating the solution with air, regenerating the characteristic purple color of the solution and the original UV signal at 355 nm while the bands at 295 and 375 nm disappeared. The same redox sequence was monitored by CD spectroscopy. The redox switching was accompanied by a dual chiroptical response as both the intensity and the wavelength of the CD maxima are significantly changing. Reduction of the quinone conjugate produced a substantial blue shift of the large CD amplitude at 350 nm of the purple oxidated state by 60 nm. The dark yellow hydroquinone which was formed spontaneously displayed a CD signal at 290 nm with approximately half intensity. NMR and chiroptical measurements showed that the oxidation and reduction steps are fast and occur quantitatively (FIGS. 112-114). The operation of this chiroptical switch was straightforward and does not require cumbersome precautions. In fact, the individual redox states were exposed to light during all experiments and did not show any sign of photochemical degradation. Then several switching cycles were conducted with this system and found no sign of signal decay after three consecutive redox sequences (FIGS. 57B-C).

It is noteworthy that this chiroptical quinone/hydroquinone switch was easily set up with inexpensive chemicals, and it exhibits thermal and photochemical stability together with distinctive color, UV and CD signal transformations that occur in less than one minute upon addition of the external stimuli. The possibility of CD and UV switching can be exploited for the determination of the absolute configuration, the enantiomeric and diastereomeric compositions, and total concentrations of chiral compounds, in particular when complicated mixtures need to be analyzed.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An analytical method comprising:
providing a sample potentially containing a chiral analyte that can exist in stereoisomeric forms;
providing a probe selected from the group consisting of quinones and analogs thereof, (hetero)aryl isocyanates and analogs thereof, and (hetero)aryl isothiocyanates and analogs thereof;
contacting the sample with the probe under conditions to permit covalent binding of the probe to the analyte, if present in the sample; and
determining, based on any binding that occurs, the absolute configuration of the analyte in the sample and/or the concentration of the analyte in the sample and/or the enantiomeric and/or the diastereomeric composition of the analyte in the sample.

2. The analytical method of claim 1, wherein the probe is a quinone of Formula I: 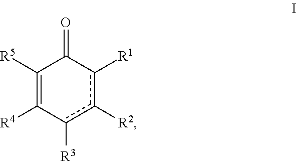
wherein:
R¹ and R³ are independently selected from the group consisting of ═O, X, halogen, —CN, —NO₂, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
R², R⁴, and R⁵ are independently selected from the group consisting of X, halogen, —CN, —C₁-C₆ alkyl, —C₁-C₆ alkoxy, —NO₂, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, and —SO₂Rₐ; wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and
X is a leaving group selected from halogen, —CN, —OR_{b}, —OC(O)R_{b}, —OS(O)₂R_{b}, —S(O)₂—O—R_{b}, —N₂⁺, —N⁺(R_{b})₃, —S⁺(R_{b})₂, and —P⁺(R_{b})₃; wherein each R_{b} is independently selected from the group consisting of -alkyl, —O-alkyl, —N-alkyl, -alkenyl, -alkynyl, -perfluoroalkyl, -perfluoroalkenyl, -perfluoroalkynyl, -aryl, -perfluoroaryl, —O-aryl, —N-aryl, —O-perfluoroaryl, —N-perfluoroaryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl;
wherein, optionally, R¹ and R², R² and R³, R³ and R⁴, and/or R⁴ and R⁵ are alternatively taken together with the carbon atoms to which they are attached to form a monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{c}, —O—C(O)R_{C}, —NHC(O)R_{c}, —NR_{c}C(O)R_{c}, —NO₂, —CN, -halogen, and —SO₂R_{c}, wherein each Re is independently Ar, alkyl, or CH₂Ar and Ar is an aryl or heteroaryl;
with the proviso that one of R¹ or R³ is ═O, and at least one of R¹, R², R³, R⁴, or R⁵ is X.

3. The analytical method of claim 2, wherein 1 or 2 of R¹, R², R³, and/or R⁴ is —NO₂.

4. The analytical method of claim 1 or 2, wherein the probe is a quinone selected from the group consisting of:

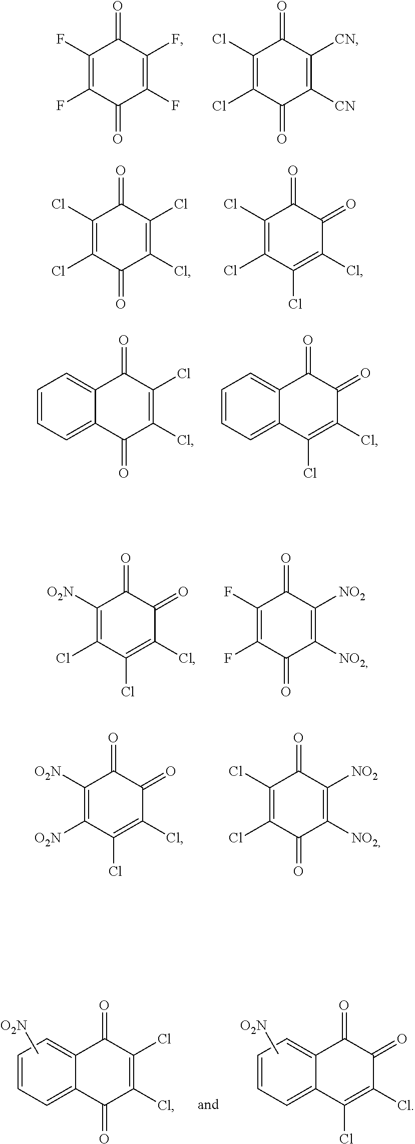

5. The analytical method of claim 1, wherein the probe is a (hetero)aryl isocyanate or (hetero)aryl isothiocyanate or an analog thereof of Formula II: 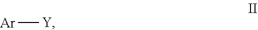
wherein:
Y is —NCO or —NCS; and
Ar is a substituted or unsubstituted aromatic or heteroaromatic chromophore.

6. The analytical method of claim 1 or claim 5, wherein the probe is a (hetero)aryl isocyanate or (hetero)aryl isothiocyanate or an analog thereof of Formula IIa: 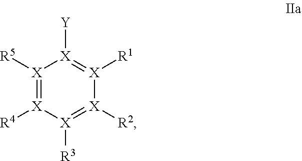
wherein:
Y is —NCO or —NCS;
each X is independently C or N; and
R¹, R², R³, R⁴, and R⁵ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ;
wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; and
wherein, optionally, R¹ and R², R² and R³, R³ and R⁴, and/or R⁴ and R⁵ are alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b}, wherein each R_{b} is independently Ar, alkyl, or CH₂Ar and Ar is an aryl or heteroaryl.

7. The analytical method of claim 6, wherein 1 or 2 of R¹, R², R³, R⁴, and R⁵ is —NO₂.

8. The analytical method of claim 1 or 5-6, wherein the probe is a (hetero)aryl isocyanate or (hetero)aryl isothiocyanate selected from:

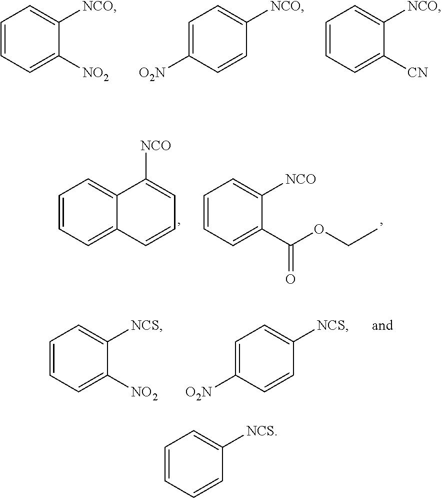

9. The analytical method of claim 1 or claim 5, wherein the probe is a (hetero)aryl isocyanate or (hetero)aryl isothiocyanate or an analog thereof of Formula IIb: 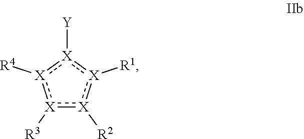
wherein:
Y is —NCO or —NCS;
each X is independently C, N, O or S; and
R¹, R², R³, and R⁴ are independently selected from the group consisting of —NCO, —NCS, a lone pair, —H, —CN, —NO₂, halogen, —C₁-C₆ alkyl, —C₁-C₆ alkoxy, —N-alkyl, —C₁-C₆ alkenyl, —C₁-C₆ alkynyl, —C₁-C₆ perfluoroalkyl, -aryl, -perfluoroaryl, -aryloxy, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, —N-heterocycloalkyl, —OH, —C(O)Rₐ, —SO₂Rₐ, and —OC(O)Rₐ;
wherein each Rₐ is independently selected from the group consisting of —H, -alkyl, —O-alkyl, —N-alkyl, -alkenyl, -alkynyl, -aryl, —O-aryl, —N-aryl, -heteroaryl, —O-heteroaryl, —N-heteroaryl, -cycloalkyl, —O-cycloalkyl, —N-cycloalkyl, -heterocycloalkyl, —O-heterocycloalkyl, and —N-heterocycloalkyl; wherein no more than one of R¹, R², R³, or R⁴, is Y; and
wherein, optionally, R¹ and R², R² and R³, and/or R³ and R⁴ is alternatively taken together with the carbon atoms to which they are attached to form monocyclic or bicyclic ring system selected from the group consisting of cycloalkyl, heterocycloalkyl, aryl, and heteroaryl, wherein the ring system is optionally substituted with one or more groups selected from -alkyl, —O-alkyl, —N-alkyl, -alkenyl, -alkynyl, —O-aryl, —O-heteroaryl, —N-aryl, —N-heteroaryl, -aryl, —C(O)R_{c}, —CO₂R_{b}, —O—C(O)R_{b}, —NHC(O)R_{b}, —NR_{c}C(O)R_{b}, —NO₂, —CN, -halogen, and —SO₂R_{b},
wherein each R_{b} is independently Ar, alkyl, or CH₂Ar and Ar is an aryl or heteroaryl.

10. The analytical method of any one of claims 1-4, wherein the probe is a quinone or analog thereof, and the analyte is selected from the group consisting of primary amines, secondary amines, diamines, amino alcohols, amino acids, amides, and combinations thereof.

11. The analytical method of any one of claims 1, and 5-9, wherein the probe is a (hetero)aryl isocyanate or analog thereof, or a (hetero)aryl isothiocyanate or analog thereof, and the analyte is selected from the group consisting of primary amines, secondary amines, diamines, amino alcohols, alcohols, diols, amino acids, thiols, and combinations thereof.

12. The analytical method of any one of claims 1-11, wherein said contacting is carried out in a solvent selected from aqueous solvents, protic solvents, aprotic solvents, organic solvents, and any combination thereof.

13. The analytical method of any one of claims 1-12, wherein said contacting is carried out in a solvent selected from chloroform, dichloromethane, acetonitrile, toluene, tetrahydrofuran, methanol, ethanol, isopropanol, water, dimethyl sulfoxide (DMSO), dimethylformamide (DMF), pentane, pentane isomers, hexane, hexane isomers, ether, dichloroethane, acetone, ethyl acetate, butanone, and mixtures of any combination thereof.

14. The analytical method of any one of claims 1-13, wherein said contacting is carried out in air.

15. The analytical method of any one of claims 1-14, wherein said contacting is carried out in an aqueous environment.

16. The analytical method of any one of claims 1-15, wherein said contacting is carried out in the presence of a base.

17. The analytical method of any one of claims 1-16, wherein said contacting is carried out in the presence of a buffer.

18. The analytical method of any one of claims 1-17, wherein said contacting is carried out for about 1 to about 300 minutes.

19. The analytical method of any one of claims 1-18, wherein said contacting is carried out under ambient conditions.

20. The analytical method of any one of claims 1-18, wherein said contacting is carried out at about 50° C. to about 100° C.

21. The analytical method of any one of claims 1-18, wherein said contacting is carried out at below about 25° C.

22. The analytical method of any one of claims 1-21, wherein the absolute configuration of the analyte in the sample is determined.

23. The analytical method of any one of claims 1-22, wherein the concentration of the analyte in the sample is determined.

24. The analytical method of any one of claims 1-23, wherein the enantiomeric composition of the analyte in the sample is determined.

25. The analytical method of any one of claims 1-24, wherein the diastereomeric composition of the analyte in the sample is determined.

26. The analytical method of any one of claims 1-25, wherein two of the absolute configuration, the concentration, the enantiomeric composition, and the diastereomeric composition of the analyte in the sample are determined.

27. The analytical method of any one of claims 1-25, wherein three of the absolute configuration, the concentration, the enantiomeric composition, and the diastereomeric composition of the analyte in the sample are determined.

28. The analytical method of any one of claims 1-25, wherein the absolute configuration, the concentration, the enantiomeric composition, and the diastereomeric composition of the analyte in the sample are determined.
